(19) United States
(12) Patent Application Publication
(10) Pub. No.: US 2024/0132970 A1
(43) Pub. Date: Apr. 25, 2024

(54) COMPOSITIONS AND METHODS FOR MAKING AND USING AN IMMORTALIZED LIBRARY

(71) Applicant: Flagship Pioneering Innovations VI, LLC, Cambridge, MA (US)

(72) Inventor: Anthony P. Shuber, Cambridge, MA (US)

(21) Appl. No.: 18/474,060

(22) Filed: Sep. 24, 2023

Related U.S. Application Data
(63) Continuation of application No. PCT/US2023/061607, filed on Jan. 30, 2023.
(60) Provisional application No. 63/304,540, filed on Jan. 28, 2022.

(51) Int. Cl. C12Q 1/6886; C12N 15/10; C12Q 1/6844

(52) U.S. Cl. CPC C12Q 1/6886; C12N 15/1082; C12Q 1/6844

(57) ABSTRACT

The invention relates generally to immortalized libraries, also referred to as archived reference samples, and their use in diagnostic methods.

### CROSS REFERENCE TO RELATED APPLICATIONS

This application is a continuation of International Application No. PCT/US2023/061607 filed Jan. 30, 2023, which claims the benefit of and priority to U.S. Provisional Patent Application No. 63/304,540, filed Jan. 28, 2022, the contents of each of which are incorporated by reference in their entirety herein.

The instant application contains a Sequence Listing which has been submitted electronically in XML file format and is hereby incorporated by reference in its entirety. Said XML copy, created on Nov. 27, 2023, is named FLG-010WOC1_SL.xml and is 2,838 bytes in size.

### FIELD OF THE INVENTION

The invention relates generally to immortalized libraries, also referred to as archived reference samples, and their use in diagnostic methods.

### BACKGROUND

In the field of diagnostics, biological samples of a variety of types are collected, then processed using diagnostic assays, to assess health conditions. Generally, by performing a diagnostic assay a biological sample is consumed at least partially and may be destroyed.

When a diagnostic assay is subjected to a clinical trial to validate its performance, a set of duplicate biological samples are necessary but may not be sufficient. The set of biological samples must originate from a statistically relevant number of sources, e.g., subjects, to enable validation of the diagnostic assay. Typically, a clinical research organization (CRO) manages the clinical trial, including gathering and maintaining the biological samples. The CRO collects a sufficient number of biological samples from a target population to successfully complete the clinical trial. Limits on both the number and the quantity of the biological samples include biological limits (since the samples typically are extracts from sources such as human subjects) and process limits (such as having sufficient materials, personnel, space, or other resources for obtaining and storing the samples). Information about the sources of the biological samples also is maintained by the CRO to ensure that the developer of the diagnostic assay is blind to any information about the sources of the biological samples.

A biological sample can take any of a variety of forms, such as a liquid biopsy (e.g., blood, urine, stool, saliva, or mucous), or a tissue biopsy, or other solid biopsy. The biological sample may be processed to extract an analyte of interest. The analyte of interest can be any molecular material found in the biological sample, such as nucleic acid molecules, protein molecules, carbohydrate molecules, blood components, bacteria, virus, or cellular components, or any combination of these. When there are multiple kinds of molecular material or multiple pieces of a kind of molecular material, such as fragments of nucleic acids, the molecular material extracted from the biological sample is an analyte having characteristics that are unique to this molecular material.

In the context of clinical trials for evaluating efficacy of a diagnostic assay, a developer of the diagnostic assay typically requests from the CRO and receives extracts taken from the biological samples. Each extract has a target amount of an analyte of interest, and a relative concentration of the analyte that may need to be preserved during processing of the extract. Depending on the nature of the biological sample and of the extract, the developer of the diagnostic assay may receive one extract or many, such as on a biochip. The developer processes these extracts using the diagnostic assay being evaluated. The developer submits its results back to the CRO. The CRO then evaluates the performance of the diagnostic assay using the information the CRO maintained about the sources of the biological samples.

Due to costs of identifying sources for a clinical trial, obtaining, and maintaining biological samples from those sources, and then managing the clinical trial, further research, development, and commercialization of a diagnostic assay is highly dependent on a successful clinical trial. Accordingly, a significant amount of investment goes into carefully designing the diagnostic assay and the protocols for the clinical trial to improve the likelihood of success of the clinical trial.

One of the risks to a successful clinical trial is the impact of an event occurring which requires reprocessing of a biological sample. For example, an error may occur while handling the biological sample or in performing the diagnostic assay. As another example, a change may be made to the diagnostic assay. When such an event occurs, the developer of the assay typically requests one or more additional extracts from one or more biological samples affected by the event from the CRO, if such extracts are available. If an additional extract for an affected biological sample is not available, results for the affected biological sample may need to be excluded from the clinical trial.

Some diagnostic assays further include computational techniques to process data obtained from assaying the biological samples. These computational techniques tend to fall in the category of “machine learning”. Machine learning generally involves using data about one set of items, called a training set, for which a property is known, such as classifications for the items, to train a computational model. The trained computational model in turn can make predictions about what that property should be for items in another set, called the target set, for which that property is not known. For example, data obtained from assaying biological samples, combined with known health conditions about the sources of the biological samples, can be used to train computational model that predicts health conditions of other sources of other biological samples.

Most techniques used in machine learning start with a selection of set of features for which values are derived from data about items in a training set. Each item in the training set has its own respective set of values corresponding to the set of features. For example, in data representing a person, the set of features representing persons may include age and location. Each person has its own respective values corresponding to these features which may be derived from data about the person.

When machine learning is used in the context of a diagnostic assay, the diagnostic assay typically includes several laboratory steps that are forms of biological and chemical process steps, and then steps that are performed using some form of equipment or sensor that generates data. These data are then inputs to computational processes that typically include training a computational model, or applying data to a trained computational model, or both.

Results obtained from applying machine learning techniques typically provide interesting insights into the diagnostic assay. For example, the results might suggest that some features are more predictive or less predictive than others. The results might suggest that some additional or different data may be helpful to improve predictive performance. The results might suggest a biological process, chemical process, equipment, or sensor, or other aspect of the diagnostic assay, may be introducing noise, error, or other aberration into the data or results derived from the data. Any of these insights might result in a desire or need to modify the diagnostic assay, or to reprocess the biological samples, or both. In such an event, the developer of the diagnostic assay may request additional extracts of the biological samples.

### SUMMARY OF THE INVENTION

The present disclosure relates, in part, to immortalized libraries, a collection of permanent reference samples (e.g., nucleic acid samples), where the collection of permanent reference samples is representative of one or more biological samples from a subject.

In a clinical trial, an immortalized library can be used to produce corresponding clone libraries multiple times without depleting the collection or losing the statistically relevant number of sources required to successfully complete the clinical trial. Additionally, an immortalized library can allow multiple clinical trials to be performed from a single collection of patient samples, thereby avoiding the substantial costs associated with recruiting patients and collecting samples and information (e.g., medical history) from such patients each time a clinical trial is conducted. This feature may be employed, for example, to evaluate a new in vitro diagnostic or to reevaluate an updated in vitro diagnostic without expending time and resources to recruit a new cohort of patients and obtain samples from the new cohort of patients each time a clinical trial is conducted. An immortalized library as contemplated herein may be particularly advantageous, for example, in instances where an in vitro diagnostic comprises an algorithm component (e.g., an artificial intelligence component) that may be updated from time to time, thereby creating a desire or necessity to reevaluate the safety and effectiveness of the in vitro diagnostic by way of a clinical trial. Conducting such a clinical trial would be considerably faster and less expensive using an immortalized library as contemplated herein instead of recruiting and obtaining samples from a new cohort of patients to conduct a clinical trial each time the algorithm is updated.

In addition to advantages for use in a clinical trial (e.g., to evaluate safety and effectiveness of an in vitro diagnostic), immortalized libraries allow for the validation and comparison of the performance of multiple different diagnostic assays based on the same biological samples.

A collection of permanent reference samples enables ongoing development, and even rapid prototyping, of a diagnostic assay in the research and development phase, by allowing continual refinement of biological processes, chemical processes, equipment, and sensors used in the assay without depleting the repository of biological samples.

A collection of permanent reference samples enables development of anew diagnostic assay using a same set of biological samples used to evaluate a previous diagnostic assay.

In one aspect, the disclosure relates to a method of emulating or conducting a human clinical study that uses a plurality of human subjects to evaluate a diagnostic parameter, comprising providing a clone library of amplified nucleic acid representative of nucleic acid from the plurality of human subjects, wherein the clone library was prepared by a process comprising obtaining the nucleic acid from the plurality of human subjects; attaching the nucleic acid or nucleic acid derived from the nucleic acid to a solid support, optionally via an adapter, to form an immortalized library of nucleic acid; and amplifying the library of nucleic acid to form the clone library of amplified nucleic acid; assaying the clone library with an assay for the diagnostic parameter to generate an output, wherein the output correlates with presence or absence of a condition in each of the plurality of human subjects; and comparing the output to a reference standard to determine sensitivity or specificity of the diagnostic parameter.

The method of claim 1, wherein the nucleic acid of the immortalized library and/or the clone library of each of the plurality of human subjects comprises a unique identifier.

In another aspect, the disclosure relates to a method of emulating or conducting a human clinical study that uses a plurality of human subjects to evaluate a diagnostic parameter, comprising providing a clone library of amplified nucleic acid from each of the plurality of human subjects, wherein at least one clone library was prepared by a process comprising obtaining nucleic acid from a human subject; attaching the nucleic acid or nucleic acid derived from the nucleic acid to a solid support, optionally via an adapter, to form an immortalized library of template nucleic acid; and amplifying the immortalized library of template nucleic acid to form the at least one clone library of amplified nucleic acid; assaying the clone libraries with an assay for the diagnostic parameter to generate an output, wherein the output correlates with presence or absence of a condition in each of the plurality of human subjects; and comparing the output to a reference standard to determine sensitivity or specificity of the diagnostic parameter.

In another aspect, the disclosure relates to a method for determining specificity or sensitivity of a diagnostic assay, comprising providing a clone library of amplified nucleic acid representative of nucleic acid from a plurality of human subjects, wherein the clone library was prepared by a process comprising obtaining the nucleic acid from the plurality of human subjects; attaching the nucleic acid or nucleic acid derived from the nucleic acid to a solid support, optionally via an adapter, to form an immortalized library of nucleic acid; and amplifying the library of nucleic acid to form the clone library of amplified nucleic acid; assaying the clone library with the diagnostic assay to generate an output, wherein the output correlates with presence or absence of a condition in each of the plurality of human subjects; and comparing the output to a reference standard to determine sensitivity or specificity of the diagnostic assay.

In certain embodiments, the nucleic acid of the immortalized library and/or the clone library of each of the plurality of human subjects comprises a unique identifier.

In another aspect, the disclosure relates to a method for determining specificity or sensitivity of a diagnostic assay, comprising providing a clone library of amplified nucleic acid from each of a plurality of human subjects, wherein at least one clone library was prepared by a process comprising obtaining nucleic acid from a human subject; attaching the nucleic acid or nucleic acid derived from the nucleic acid to a solid support, optionally via an adapter, to form an immortalized library of template nucleic acid; and amplifying the immortalized library of template nucleic acid to form the at least one clone library of amplified nucleic acid; assaying the clone libraries with the diagnostic assay to generate an output, wherein the output correlates with presence or absence of a condition in each of the plurality of human subjects; and comparing the output to a reference standard to determine sensitivity or specificity of the diagnostic assay.

In certain embodiments, the nucleic acid comprises cell-free nucleic acid. In certain embodiments, the cell-free nucleic acid is cell-free DNA. In certain embodiments, the nucleic acid is attached to the solid support via the adapter.

In certain embodiments, the adapter comprises a moiety that binds to the solid support. In certain embodiments, the adapter comprises the solid support.

In certain embodiments, the method further comprises repairing ends of the nucleic acid prior to attaching the adapter.

In certain embodiments, the method further comprises selectively deaminating cytosine residues. In certain embodiments, the step of selectively deaminating cytosine residues is performed prior to attaching the adapter. In certain embodiments, the step of selectively deaminating cytosine residues is performed after attaching the adapter. In certain embodiments, the step of selectively deaminating cytosine residues is performed after attaching the nucleic acid to the solid support. In certain embodiments, the step of selectively deaminating cytosine residues comprises a bisulfite conversion step. In certain embodiments, the bisulfite conversion step is performed prior to attaching the adapter. In certain embodiments, the step of selectively deaminating cytosine residues comprises an enzymatic conversion step. In certain embodiments, the enzymatic conversion step is selected from TET2 oxidation of cytosines and APOBEC conversion of cytosines. In certain embodiments, the enzymatic conversion step is performed after attaching the adapter. In certain embodiments, the enzymatic conversion step is performed after attaching the nucleic acid to the solid support.

In certain embodiments, the adapter is attached by ligation. In certain embodiments, the adapter is attached by nucleic acid amplification of the nucleic acid using a primer comprising the adapter.

In certain embodiments, the solid support is selected from a bead, a slide, a membrane, a planar surface, a microtiter plate, a filter, a test strip, a slide, a cover slip, and a test tube. In certain embodiments, the moiety that binds to a solid support is selected from biotin and streptavidin. In certain embodiments the solid support or the moiety that binds to the solid support is connected to the adapter via a linker. In certain embodiments the linker is a TEG linker.

In certain embodiments, the adapter comprises one or more of a flow cell binding site, an index, a unique molecular identifier (UMI), and a sequencing binding site.

In certain embodiments, the condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments neurodegenerative disease is one of Alzheimer's disease, Parkinson's disease, amyotrophic lateral sclerosis (ALS), frontotemporal dementia (FTD).

In certain embodiments, the diagnostic parameter comprises a methylation state of one or more nucleotides in the cell-free DNA or the cell-free RNA or the presence of a mutation in the cell-free DNA or the cell-free RNA.

In certain embodiments, the output comprises a fluorescent signal.

In certain embodiments, a diagnostic device is used to evaluate the diagnostic parameter.

In certain embodiments, the method further comprises recovering the immortalized library.

In certain embodiments, the method further comprises assaying a second diagnostic parameter, comprising the steps of (1) assaying a second clone library with an assay for the second diagnostic parameter to generate a second output, wherein the second clone library was generated by amplifying the immortalized library, wherein the second output correlates with presence or absence of the condition or a second condition in each of the plurality of human subjects; and (2) comparing the second output to a second reference standard to determine sensitivity or specificity of the second diagnostic parameter. In certain embodiments, the method further comprises repeating steps (1) and (2) with at least one additional clone library to determine sensitivity or specificity for at least one additional diagnostic parameter. In certain embodiments, the second diagnostic parameter is the same as the diagnostic parameter. In certain embodiments, the second diagnostic parameter is different from the diagnostic parameter. In certain embodiments, the at least one additional diagnostic parameter is the same as the diagnostic parameter and the second diagnostic parameter. In certain embodiments, the second output is the same as the output. In certain embodiments, the second output is different from the output. In certain embodiments, the nucleic acid is cell-free nucleic acid and the second diagnostic parameter or the at least one additional diagnostic parameter comprises a methylation state of one or more nucleotides in the cell-free nucleic acid or the presence of a mutation in the cell-free nucleic acid. In certain embodiments, the second output comprises a fluorescent signal. In certain embodiments, the first and second diagnostic parameters are the same, and a diagnostic device is used to evaluate the first and second diagnostic parameters. In certain embodiments, the diagnostic device is changed (e.g., improved) between evaluation of the first diagnostic parameter and evaluation of the second diagnostic parameter.

In certain embodiments, the second condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments, the neurodegenerative disease is one of Alzheimer's disease, Parkinson's disease, amyotrophic lateral sclerosis (ALS), frontotemporal dementia (FTD).

In certain embodiments, the second clone library is generated at least 4 weeks, at least 4 months, at least 6 months, at least 1 year, at least 2 years, at least 5 years, or at least 10 years after the clone library is generated. In certain embodiments, at least one additional clone library is generated at least 4 weeks, at least 4 months, at least 6 months, at least 1 year, at least 2 years, at least 5 years, or at least 10 years after the second clone library is generated.

In another aspect, the disclosure relates to a method of assessing the correlation of a potential diagnostic parameter with a condition in a plurality of human subjects, comprising providing a clone library of amplified nucleic acid from each of the plurality of human subjects, wherein the clone library was prepared by a process comprising obtaining nucleic acid from the plurality of human subjects; attaching the nucleic acid or nucleic acid derived from the nucleic acid to a solid support, optionally via an adapter, to form an immortalized library of template nucleic acid; and amplifying the library of template nucleic acid to form the clone library of amplified nucleic acid; assaying the clone library with an assay for the diagnostic parameter to generate an output; determining whether the output correlates with presence or absence of a condition in each of the plurality of human subjects.

In another aspect, the disclosure relates to a method of assessing the correlation of a potential diagnostic parameter with a condition in a plurality of human subjects, comprising providing a clone library of amplified nucleic representative of nucleic acid from the plurality of human subjects, wherein the clone library was prepared by a process comprising obtaining nucleic acid from the plurality of human subjects attaching the nucleic acid or nucleic acid derived from the nucleic acid to a solid support, optionally via an adapter, to form an immortalized library of template nucleic acid; and amplifying the library of template nucleic acid to form the clone library of amplified nucleic acid; assaying the clone library with an assay for the diagnostic parameter to generate an output; determining whether the output correlates with presence or absence of a condition in each of the plurality of human subjects.

In certain embodiments, the nucleic acid of the immortalized library and/or the clone library of each of the plurality of human subjects comprises a unique identifier.

In certain embodiments, the nucleic acid is attached to the solid support via the adapter. In certain embodiments, the adapter comprises a moiety that binds to the solid support. In certain embodiments, the adapter comprises the solid support.

In certain embodiments, the method further comprises repairing ends of the nucleic acid or the nucleic acid derived from the nucleic acid prior to attaching the adapter. In certain embodiments, the method further comprises repairing ends of the nucleic acid prior to attaching the adapter.

In certain embodiments, the method further comprises selectively deaminating cytosine residues. In certain embodiments, the step of selectively deaminating cytosine residues is performed prior to attaching the adapter. In certain embodiments, the step of selectively deaminating cytosine residues is performed after attaching the adapter. In certain embodiments, the step of selectively deaminating cytosine residues is performed after attaching the nucleic acid to the solid support. In certain embodiments, the step of selectively deaminating cytosine residues comprises a bisulfite conversion step. In certain embodiments, the bisulfite conversion step is performed prior to attaching the adapter. In certain embodiments, the step of selectively deaminating cytosine residues comprises an enzymatic conversion step. In certain embodiments, the enzymatic conversion step is selected from TET2 oxidation of cytosines and APOBEC conversion of cytosines. In certain embodiments, the enzymatic conversion step is performed after attaching the adapter. In certain embodiments, the enzymatic conversion step is performed after attaching the nucleic acid to the solid support.

In certain embodiments, the adapter is attached by ligation. In certain embodiments, the adapter is attached by nucleic acid amplification of the nucleic acid using a primer comprising the adapter. In certain embodiments, the solid support is selected from a bead, a slide, a membrane, a planar surface, a microtiter plate, a filter, a test strip, a slide, a cover slip, and a test tube. In certain embodiments, the moiety that binds to the solid support is selected from biotin and streptavidin. In certain embodiments, the solid support or the moiety that binds to the solid support is connected to the adapter via a linker. In certain embodiments, the linker is a TEG linker. In certain embodiments, the adapter comprises one or more of a flow cell binding site, an index, a unique molecular identifier (UMI), and a sequencing binding site.

In certain embodiments, the condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments, the neurodegenerative disease is one of Alzheimer's disease, Parkinson's disease, amyotrophic lateral sclerosis (ALS), frontotemporal dementia (FTD).

In certain embodiments, the nucleic acid comprises cell-free nucleic acid and the diagnostic parameter comprises a methylation state of one or more nucleotides in the cell-free nucleic acid or the presence of a mutation in the cell-free nucleic acid.

In certain embodiments, the output comprises a fluorescent signal.

In certain embodiments, a diagnostic device is used to evaluate the diagnostic parameter.

In certain embodiments, the method further comprises recovering the immortalized library.

In certain embodiments, the method further comprises assaying a second diagnostic parameter, comprising the steps of (1) assaying a second clone library with an assay for the second diagnostic parameter to generate a second output, wherein the second clone library was generated by amplifying the immortalized library, determining whether the output correlates with presence or absence of the condition or a second condition in each of the plurality of human subjects. In certain embodiments, the method further comprises repeating steps (1) and (2) with at least one additional clone library to determine whether the output correlates with presence or absence of the condition, the second condition, or at least one addition condition for at least one additional diagnostic parameter. In certain embodiments, the second diagnostic parameter is the same as the diagnostic parameter. In certain embodiments, the second diagnostic parameter is different from the diagnostic parameter. In certain embodiments, the at least one additional diagnostic parameter is the same as the diagnostic parameter and the second diagnostic parameter. In certain embodiments, the second output is the same as the output. In certain embodiments, the second output is different from the output. In certain embodiments, the nucleic acid comprises cell-free nucleic acid and the second diagnostic parameter or the at least one additional diagnostic parameter comprises a methylation state of one or more nucleotides in the cell-free nucleic acid or the presence of a mutation in the cell-free nucleic acid. In certain embodiments, the second output comprises a fluorescent signal.

In certain embodiments, the second condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments, the neurodegenerative disease is one of Alzheimer's disease, Parkinson's disease, amyotrophic lateral sclerosis (ALS), frontotemporal dementia (FTD).

In certain embodiments, the second clone library is generated at least 4 weeks, at least 4 months, at least 6 months, at least 1 year, at least 2 years, at least 5 years, or at least 10 years after the clone library is generated. In certain embodiments, the at least one additional clone library is generated at least 4 weeks, at least 4 months, at least 6 months, at least 1 year, at least 2 years, at least 5 years, or at least 10 years after the second clone library is generated.

In another aspect, the disclosure relates to a method of emulating or conducting a human clinical study that uses a plurality of human subjects to evaluate a diagnostic parameter, comprising providing a clone library of amplified nucleic acid from each of the plurality of human subjects, wherein at least one clone library was prepared by a process comprising obtaining nucleic acid from a human subject; forming a immortalized library of template nucleic acid, wherein the immortalized library comprises a component or means for recovering the template nucleic acid; and amplifying the immortalized library of template nucleic acid to form the at least one clone library of amplified nucleic acid; assaying the clone libraries with an assay for the diagnostic parameter to generate an output, wherein the output correlates with presence or absence of a condition in each of the plurality of human subjects; and comparing the output to a reference standard to determine sensitivity or specificity of the diagnostic parameter.

In another aspect, the disclosure relates to a method for determining specificity or sensitivity of a diagnostic assay, comprising providing a clone library of amplified nucleic acid representative of nucleic acid from a plurality of human subjects, wherein the clone library was prepared by a process comprising obtaining the nucleic acid from the plurality of human subjects; forming a immortalized library of template nucleic acid, wherein the immortalized library comprises a component or means for allowing for the recovery of the template nucleic acid; and amplifying the library of nucleic acid to form the clone library of amplified nucleic acid; assaying the clone library with the diagnostic assay to generate an output, wherein the output correlates with presence or absence of a condition in each of the plurality of human subjects; and comparing the output to a reference standard to determine sensitivity or specificity of the diagnostic assay.

In certain embodiments, the component for the recovery of the template nucleic acid comprises a solid support or a moiety that binds to a solid support.

In certain embodiments, the component for the recovery of the template nucleic acid comprises a detectable (e.g., fluorescent) moiety.

In certain embodiments, the nucleic acid of the immortalized library and/or the clone library of each of the plurality of human subjects comprises a unique identifier.

In certain embodiments, the nucleic acid is attached to the solid support via the adapter. In certain embodiments, the adapter comprises a moiety that binds to the solid support. In certain embodiments, the adapter comprises the solid support.

In certain embodiments, the method further comprises repairing ends of the nucleic acid or the nucleic acid derived from the nucleic acid prior to attaching the adapter. In certain embodiments, the method further comprises repairing ends of the nucleic acid prior to attaching the adapter.

In certain embodiments, the method further comprises selectively deaminating cytosine residues. In certain embodiments, the step of selectively deaminating cytosine residues is performed prior to attaching the adapter. In certain embodiments, the step of selectively deaminating cytosine residues is performed after attaching the adapter. In certain embodiments, the step of selectively deaminating cytosine residues is performed after attaching the nucleic acid to the solid support. In certain embodiments, the step of selectively deaminating cytosine residues comprises a bisulfite conversion step. In certain embodiments, the bisulfite conversion step is performed prior to attaching the adapter. In certain embodiments, the step of selectively deaminating cytosine residues comprises an enzymatic conversion step. In certain embodiments, the enzymatic conversion step is selected from TET2 oxidation of cytosines and APOBEC conversion of cytosines. In certain embodiments, the enzymatic conversion step is performed after attaching the adapter. In certain embodiments, the enzymatic conversion step is performed after attaching the nucleic acid to the solid support.

In certain embodiments, the adapter is attached by ligation. In certain embodiments, the adapter is attached by nucleic acid amplification of the nucleic acid using a primer comprising the adapter. In certain embodiments, the solid support is selected from a bead, a slide, a membrane, a planar surface, a microtiter plate, a filter, a test strip, a slide, a cover slip, and a test tube. In certain embodiments, the moiety that binds to the solid support is selected from biotin and streptavidin. In certain embodiments, the solid support or the moiety that binds to the solid support is connected to the adapter via a linker. In certain embodiments, the linker is a TEG linker. In certain embodiments, the adapter comprises one or more of a flow cell binding site, an index, a unique molecular identifier (UMI), and a sequencing binding site.

In certain embodiments, the condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments, the neurodegenerative disease is one of Alzheimer's disease, Parkinson's disease, amyotrophic lateral sclerosis (ALS), frontotemporal dementia (FTD).

In certain embodiments, the nucleic acid comprises cell-free nucleic acid and the diagnostic parameter comprises a methylation state of one or more nucleotides in the cell-free nucleic acid or the presence of a mutation in the cell-free nucleic acid.

In certain embodiments, the output comprises a fluorescent signal.

In certain embodiments, a diagnostic device is used to evaluate the diagnostic parameter.

In certain embodiments, the method further comprises recovering the immortalized library.

In certain embodiments, the method further comprises assaying a second diagnostic parameter, comprising the steps of (1) assaying a second clone library with an assay for the second diagnostic parameter to generate a second output, wherein the second clone library was generated by amplifying the immortalized library, determining whether the output correlates with presence or absence of the condition or a second condition in each of the plurality of human subjects. In certain embodiments, the method further comprises repeating steps (1) and (2) with at least one additional clone library to determine whether the output correlates with presence or absence of the condition, the second condition, or at least one addition condition for at least one additional diagnostic parameter. In certain embodiments, the second diagnostic parameter is the same as the diagnostic parameter. In certain embodiments, the second diagnostic parameter is different from the diagnostic parameter. In certain embodiments, the at least one additional diagnostic parameter is the same as the diagnostic parameter and the second diagnostic parameter. In certain embodiments, the second output is the same as the output. In certain embodiments, the second output is different from the output. In certain embodiments, the nucleic acid comprises cell-free nucleic acid and the second diagnostic parameter or the at least one additional diagnostic parameter comprises a methylation state of one or more nucleotides in the cell-free nucleic acid or the presence of a mutation in the cell-free nucleic acid. In certain embodiments, the second output comprises a fluorescent signal.

In certain embodiments, the second condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments, the neurodegenerative disease is one of Alzheimer's disease, Parkinson's disease, amyotrophic lateral sclerosis (ALS), frontotemporal dementia (FTD).

In certain embodiments, the second clone library is generated at least 4 weeks, at least 4 months, at least 6 months, at least 1 year, at least 2 years, at least 5 years, or at least 10 years after the clone library is generated. In certain embodiments, the at least one additional clone library is generated at least 4 weeks, at least 4 months, at least 6 months, at least 1 year, at least 2 years, at least 5 years, or at least 10 years after the second clone library is generated.

In another aspect, the disclosure relates to a method for simultaneously analyzing a bio-response in nucleic acid from a plurality of human subjects, comprising providing a clone library of amplified nucleic acid representative of nucleic acid from a plurality of human subjects, wherein the clone library was prepared by a process comprising obtaining nucleic acid from the plurality of human subjects; attaching the nucleic acid or nucleic acid derived from the nucleic acid to a solid support, optionally via an adapter, to form an immortalized library of template nucleic acid; and amplifying the immortalized library of template nucleic acid to form the clone library of amplified nucleic acid; assaying the clone library for the bio-response, wherein the bio-response is indicative of a clinical outcome assessment.

In another aspect, the disclosure relates to a system for simultaneously analyzing a bio-response in nucleic acid from a plurality of human subjects, comprising providing a clone library of amplified nucleic acid representative of nucleic acid from a plurality of human subjects, wherein the clone library was prepared by a process comprising obtaining nucleic acid from the plurality of human subjects; attaching the nucleic acid or nucleic acid derived from the nucleic acid to a solid support, optionally via an adapter, to form an immortalized library of template nucleic acid; and amplifying the immortalized library of template nucleic acid to form the clone library of amplified nucleic acid; assaying the clone library for the bio-response, wherein the bio-response is indicative of a clinical outcome assessment.

In certain embodiments, the nucleic acid is cell-free nucleic acid. In certain embodiments, the cell-free nucleic acid is cell-free DNA.

In certain embodiments, at least some of the plurality of human subjects were suffering from pre-clinical stage cancer, stage I cancer, stage II cancer, stage III cancer or stage IV cancer.

In certain embodiments, the clinical outcome assessment is one or more of symptomatic progression, pain intensity, and overall response rate.

In another aspect, the disclosure relates to a system for simultaneously analyzing a bio-response in nucleic acid from a plurality of human subjects, the system comprising (a) a processor; (b) a data storage comprising sequence information derived from one or more clone libraries obtained from the plurality of human subjects, wherein the one or more clone library was prepared by a process comprising obtaining nucleic acid from the plurality of human subjects; attaching the nucleic acid or nucleic acid derived from the nucleic acid to a solid support, optionally via an adapter, to form an immortalized library of template nucleic acid; and amplifying the immortalized library of template nucleic acid to form the clone library of amplified nucleic acid; (c) a non-transitory computer readable medium comprising instructions that, when executed by the processor, cause the processor to: analyze the sequence information for the bio-response, wherein the bio-response is indicative of a clinical outcome assessment.

In another aspect, the disclosure relates to a method of making an immortalized nucleic acid library, the method comprising obtaining a plurality of fragments of nucleic acid or nucleic acid derived from the nucleic acid from one or more human subjects; attaching an adapter to the nucleic acid or the nucleic acid derived from nucleic acid; wherein the adapter is bound to a solid support or comprises a moiety capable of binding to a solid support; wherein, if the adapter is not bound to a solid support, the method further comprises binding the adapter to the solid support.

In certain embodiments, the method further comprises repairing ends of the nucleic acid or the nucleic acid derived from the nucleic acid prior to attaching the adapter.

In certain embodiments, the nucleic acid is cell-free nucleic acid. In certain embodiments, the cell-free nucleic acid is cell-free DNA.

In certain embodiments, the method further comprises selectively deaminating cytosine residues. In certain embodiments, the step of selectively deaminating cytosine residues is performed prior to attaching the adapter. In certain embodiments, the step of selectively deaminating cytosine residues is performed after attaching the adapter. In certain embodiments, the step of selectively deaminating cytosine residues is performed after attaching the nucleic acid to the solid support. In certain embodiments, the step of selectively deaminating cytosine residues comprises a bisulfite conversion step. In certain embodiments, the bisulfite conversion step is performed prior to attaching the adapter. In certain embodiments, the step of selectively deaminating cytosine residues comprises an enzymatic conversion step. In certain embodiments, the enzymatic conversion step is selected from TET2 oxidation of cytosines and APOBEC conversion of cytosines. In certain embodiments, the enzymatic conversion step is performed after attaching the adapter. In certain embodiments, the enzymatic conversion step is performed after binding the adapter to the solid support.

In certain embodiments, the adapter is attached by ligation. In certain embodiments, the adapter is a Y adapter. In certain embodiments, the adapter is attached by nucleic acid amplification of the cell-free nucleic acid using a primer comprising the adapter. In certain embodiments, the solid support is selected from a bead, a slide, a membrane, a planar surface, a microtiter plate, a filter, a test strip, a slide, a cover slip, and a test tube. In certain embodiments, the moiety capable of binding to a solid support is selected from biotin and streptavidin. In certain embodiments, the solid support or the moiety capable of binding to a solid support is connected to the adapter via a linker. In certain embodiments, the linker is a TEG linker. In certain embodiments, the adapter comprises one or more of a flow cell binding site, an index, a unique molecular identifier (UMI), and a sequencing binding site.

In certain embodiments, the method further comprises storing the immortalized library in a storage medium. In certain embodiments, the storage medium preserves the integrity of the immortalized library, wherein the integrity of the immortalized library is measured by maintenance of concordance of methylated variants over at least 6 weeks, at least 6 months, at least 1 year, at least 2 years, at least 5 years, or at least 10 years.

In certain embodiments, an immortalized nucleic acid library is produced using any method as described herein.

In another aspect, the disclosure relates to an immortalized nucleic acid library, the library comprising a plurality of nucleic acid molecules comprising nucleic acid or nucleic acid derived from the nucleic acid from a subject; wherein each of the plurality of nucleic acid molecules is attached to a solid support.

In certain embodiments, each nucleic acid molecule is attached to the solid support via an adapter. In certain embodiments, the nucleic acid is cell-free nucleic acid. In certain embodiments, the cell-free nucleic acid is cell-free DNA.

In certain embodiments, cytosine residues have been selectively deaminated. In certain embodiments, the cytosine residues have been selectively deaminated by a bisulfite conversion step or an enzymatic conversion step. In certain embodiments, the enzymatic conversion step is selected from TET2 oxidation of cytosines and APOBEC conversion of cytosines.

In certain embodiments, the solid support is selected from a bead, a slide, a membrane, a planar surface, a microtiter plate, a filter, a test strip, a slide, a cover slip, and a test tube.

In certain embodiments, each of the plurality of nucleic acid molecules comprises biotin and streptavidin. In certain embodiments, each of the plurality of nucleic acid molecules is attached to a solid support via a linker. In certain embodiments, the linker is a TEG linker.

In certain embodiments, the adapter comprises one or more of a flow cell binding site, an index, a unique molecular identifier (UMI), and a sequencing binding site.

In certain embodiments, a method of amplifying an immortalized nucleic acid library to produce a clone library is provided, the method comprising amplifying the nucleic acid library to produce a clone library of amplified nucleic acid.

In certain embodiments, the nucleic acid comprises cell-free DNA or cell-free RNA, and the immortalized library comprises information about methylation variants in the cell-free DNA. In certain embodiments, concordance of the methylation variants is maintained as compared to an amplification using a non-immortalized nucleic acid library.

In certain embodiments, the method further comprises analyzing a feature of the clone library to generate a classifier score. In certain embodiments, concordance of the classifier score is maintained as compared to an amplification using a non-immortalized nucleic acid library. In certain embodiments, concordance of the methylation variants is maintained over at least 5 amplification reactions, at least 10 amplification reactions, or at least 20 amplification reactions. In certain embodiments, the concordance is determined by measuring at least one of qPCR, iSeq, and NovaSeq. In certain embodiments, one or more metrics of percentage of methylated GC, coverage, alignment rate, unique rate, and on target rate of the clone library are comparable to an amplification using a non-immortalized nucleic acid library. In certain embodiments, the method further comprises recovering the immortalized library after the amplifying step.

In certain embodiments, a method of performing a diagnostic assay is provided, the method comprising amplifying the immortalized library to form a clone library of amplified nucleic acid; assaying the clone library with an assay for a diagnostic parameter to generate an output, wherein the output correlates with presence or absence of a condition in the plurality of human subjects. In certain embodiments, the method further comprises recovering the immortalized library after the amplifying step.

In certain embodiments, the nucleic acid is cell-free DNA or cell-free RNA and the diagnostic parameter comprises methylation status of at least one genomic site on the cell-free DNA or cell-free RNA.

In certain embodiments, the condition is selected from cancer, inflammatory disease, neurodegenerative disease, autoimmune disorder, neuromuscular disease, metabolic disorder, cardiac disease, or fibrotic disease. In certain embodiments, the neurodegenerative disease is one of Alzheimer's disease, Parkinson's disease, amyotrophic lateral sclerosis (ALS), frontotemporal dementia (FTD).

In another aspect, the disclosure relates to a method of treating a subject having a condition, the method comprising, administering to the subject a therapeutic agent suitable for treating the condition, wherein the subject was diagnosed with the condition using a method comprising the following steps: (1) obtaining a plurality of fragments of nucleic acid or nucleic acid derived from nucleic acid from the subject; (2) attaching an adapter to the nucleic acid or the nucleic acid derived from the nucleic acid, wherein the adapter is bound to a solid support, to form an immortalized library; (3) amplifying the immortalized library to form a clone library of amplified nucleic acid; (4) assaying the clone library with an assay for a diagnostic parameter to generate an output, wherein the output correlates with presence of the condition in the subject.

In another aspect, the disclosure relates to a method of emulating or conducting a human clinical study that uses a plurality of human subjects to evaluate a diagnostic parameter, comprising providing a clone library of amplified nucleic acid representative of nucleic acid from each of the plurality of human subjects, wherein the clone library was prepared by a process comprising obtaining the nucleic acid from a human subject; attaching the nucleic acid or nucleic acid derived from the nucleic acid to an adapter, and amplifying the nucleic acid in excess (e.g., 5 to 15 times) to form an immortalized library of nucleic acid; and amplifying the immortalized library of nucleic acid to form the clone library of amplified nucleic acid; assaying the clone library with an assay for the diagnostic parameter to generate an output, wherein the output correlates with presence or absence of a condition in each of the plurality of human subjects; and comparing the output to a reference standard to determine sensitivity or specificity of the diagnostic parameter.

In certain embodiments, the nucleic acid obtained from the human subject of the plurality of human subjects is obtained from a sample. In embodiments of any of the foregoing methods or systems, the method can be repeated without any additional sample manipulation or intervention, thereby allowing any clinical trial or other investigation to be performed in an iterative manner with results representative of the original results associated with the human subject or plurality of human subjects without acquiring a new sample.

In any of the embodiments described herein, the method can include adding (“spiking in”) an amount of a reference nucleic acid to allow for quantifying the amount of an analyte present in a sample.

In any of the embodiments described herein, the method can include subjecting the library (library, original library, immortalized library, AReS library), to a selective enrichment step, for example, hybrid capture.

These and other aspects and features of the invention are described in the following detailed description and claims.

### DESCRIPTION OF THE DRAWINGS

The invention can be more completely understood with reference to the following drawings.

FIG. 1 represents a method disclosed herein. A template nucleic acid from an immortalized library (“immortalized product”) corresponds to the sequence of the nucleic acid in a sample from a subject. The nucleic acid in the template library is attached to a solid surface by a ligand attached at of the nucleic acid termini. The nucleic acid in the template library is copied, e.g., by PCR amplification. The nucleic acid template is recovered (lower right) and the copy is used to produce a clone library (upper right).

FIG. 2A shows three strategies of the method disclosed herein for producing a template library and copying it.

FIG. 2B shows a specific embodiment of the first method described in FIG. 2A, which shows a schematic for a method of producing a template library (AReS library) using excess amplification of an indexed library from a patient sample.

FIG. 3 provides a schematic diagram showing the construction of an immortalized library using bisulfite conversion of cytosines to preserve methylation status and biotinylated primers. Cell-free DNA is obtained from a sample and denatured. Single-stranded DNA is subjected to bisulfite conversion to preserve methylation status. Adaptase technology is used to add adapters to the cell-free DNA, and the cell-free DNA is copied (“extension”). An additional adapter is ligated to the cell-free DNA (“ligation”). The left side (“standard PCR”) shows an embodiment in which standard primers are used in production of a non-immortalized library (e.g., a typical sequencing library). The right side (“immortalized PCR”) shows an embodiment in which biotinylated primers (that can be bound to beads or other solid support) are used to produce an immortalized library which can be amplified as needed.

FIG. 4 provides a schematic diagram showing the construction of an immortalized library using Y adapters, coupled with enzymatic conversion of cytosines to preserve methylation status. In this embodiment, cell-free DNA is obtained from a sample and subjected to blunt-end repair. Y adapters are added to the cell-free DNA, and the cell-free DNA is copied (“extension”). Template nucleic acid with adapters is subjected to enzymatic conversion (TET2 oxidation of cytosines followed by APOBEC conversion of cytosines to uracils to preserve methylation status. Biotinylated primers (that can be bound to beads or other solid support) are used to produce an immortalized library which can be amplified as needed.

FIG. 5 provides a schematic diagram showing an embodiment in which biotinylated Y adapters are added to cell-free DNA, the template DNA is bound to beads via the biotin, and the template DNA is subject to on bead enzymatic conversion to preserve information about methylation status.

FIG. 6 shows a schematic diagram showing exemplary adapters suitable for use with the immortalized libraries provided herein. Creation of libraries for next generation sequencing technologies use adapters as shown at the top of the figure “Current schematic”, including a flow cell binding site, an index and a sequence binding site. The flow cell binding site allows the nucleic acid molecule to attach to the flow cell for sequencing. The index sequence, sometimes referred to as 15 and i7, act as barcodes to allow multiplexing of samples. The sequence binding site is used to initiate sequencing. The middle portion of the figure (“Primer Immortalization Methods 1 and 2”) shows an adapter suitable for use in the first two methods described in FIG. 2. In this embodiment, the adaptor includes the portions of the adaptor described above, and, in addition, includes a UMI (unique molecular identifier) as described herein. Further, the adapter is attached to a biotin tag via a TEG linker. The lower portion of the figure (“Seq binding site Immortalization Methods 3”) shows a biotinylated adapter and a standard primer suitable for use in the third method described in FIG. 2. In this embodiment, a biotinylated adaptor comprising a sequence binding site and a UMI and a standard primer comprising a flow cell binding site, an index and a sequence binding site are used to generate an immortalized library.

FIG. 7A provides a schematic showing an exemplary use of an immortalized library in a clinical trial context and an exemplary cost-savings analysis.

FIG. 7B illustrates an example computer included in a system for analyzing a nucleic acid.

FIG. 8 provides graphs showing that PCR products of different sizes were found depending upon the amount of input (1 ng, 5 ng, 10 ng, or 20 ng DNA) and the number of cycles (5, 10, 15, 20).

FIG. 9 provides annotated histograms for the 1 ng input trial, showing peaks of different-sized products. As shown, at cycles of 15 or more, “bubble product”, begins to form. Bubble product is depicted over the third peak as a spaced-apart DNA schematic. Bubble product is formed when there is insufficient primer. It does not impact sequencing quality but it is indicative of biased amplification.

FIG. 10 provides line graphs showing that the maximum total yield for an AReS library was achieved using 3× (30 μM) primer concentration (yield was lowest with 1× primer concentration, increased with 2× primer concentration, and highest with 3× or 4× primer concentration).

FIG. 11 is a graph showing that inputs greater than 1 ng met median target coverage (100× minimum median coverage).

FIG. 12 is a graph showing that median target coverage was similar over PCR cycles, indicating that PCR cycle number has little impact on median target coverage.

FIG. 13 is a bar graph showing that the percentage of unique reads increases with initial AReS input and begins to plateau at about 40 ng of input.

FIG. 14 provides bar graphs showing that an AReS library created with 20 ng input showed a lower fraction unique at 10M than did the original library (left hand graph). When additional reads are produced through additional sequencing (middle graph), the AReS library contained more unique reads than did the original library (right hand graph).

FIG. 15 provides bar graphs showing that consistency of classifier scores was seen across inputs.

FIG. 16 provides bar graphs showing that consistency of classifier scores was seen across PCR cycles.

FIG. 17 provides bar graphs showing amplified samples demonstrated concordant classifier scores across replicates. For each patient sample (SA_ID), the control (standard process) is shown to the left (light colored bar) and the three (3) AReS replicate samples are shown to the right (dark bars 1, 2, and 3).

FIG. 18 provides concordance plots showing that the PyMHap metrics of AReS libraries are highly concordant to those of original libraries across all of the CGIs of a 4,059 CGI panel (AReS scores on y-axis and original library scores on x-axis).

FIG. 19 provides a plot showing cancer yes/no (CYN) scores measure from an original library and 3 AReS library replicates.

FIG. 20A provides a schematic of unique molecular indices (UMIs) incorporated into bisulfite treated cfDNA libraries. These UMIs contained random 9 bp sequences which barcoded individual strands of cfDNA.

FIG. 20B provides a flowchart showing that standard libraries and AReS libraries were generated from the original libraries and both sets were then enriched with the 4,059 hybrid capture probe set prior to sequencing.

FIG. 21A provides a Venn diagram showing that across the 8 samples described in the flowchart of FIG. 20B, ˜77% of reads were common between the original and AReS libraries.

FIG. 21B provides a Venn diagram showing that original library replicates (Rep 1) and second replicates (Rep 2) also shared ˜77% of reads.

### DETAILED DESCRIPTION

Various features and aspects of the invention are discussed in more detail below.

The present disclosure relates, in part, to immortalized libraries, a collection of permanent reference samples (e.g., nucleic acid samples), where the collection of permanent reference samples is representative of one or more a biological samples from a subject.

In a clinical trial, an immortalized library can be used to produce corresponding clone libraries multiple times without depleting the collection or losing the statistically relevant number of sources required to successfully complete the clinical trial.

In addition to advantages for use in a clinical trial, immortalized libraries allow for the validation and comparison of the performance of multiple different diagnostic assays based on the same biological samples.

A collection of permanent reference samples enables ongoing development, and even rapid prototyping, of a diagnostic assay in the research and development phase, by allowing continual refinement of biological processes, chemical processes, equipment, and sensors used in the assay without depleting the repository of biological samples.

A collection of permanent reference samples enables development of a new diagnostic assay using a same set of biological samples used to evaluate a previous diagnostic assay.

In some implementations, each immortalized library also can include information about the sample. This information can include, but is not limited to, information uniquely identifying the sample from among other samples, information about the source from which the sample was obtained, or any combination of such information. The information can be in the form of a label affixed to a vessel containing the permanent reference sample. In some implementations, the immortalized library is stored in a container, and a label is affixed to the container. In some implementations, the label is a molecule secured to the same medium to which molecular material from the sample is secured. The label can be in the form of a barcode, molecule, or any other device which, when affixed to the container or secured to the medium for the permanent reference sample, allows information encoded in the label to be retrieved. For example, all data that is stored by a CRO about a sample, can be encoded into a label for that sample. In the event that data about a sample stored in a computer system by a CRO is lost, that data can be retrieved from the permanent reference sample.

An example implementation of a process for creating a permanent reference sample from biopsy comprises extracting cell-free nucleic acid, wherein the cell-free nucleic acid comprises a mixture of single stranded nucleic acid and double stranded nucleic acid, ligating (biotinylated Y) adapters to each strand of the cell-free nucleic acids to produce tagged (biotinylated) cell-free nucleic acid, and separating the strands of cell-free nucleic acid and binding them to a solid medium to produce template cell-free nucleic acid molecules, wherein the template cell-free nucleic acid molecules are capable being converted by DNA polymerase to cell-free nucleic acid copies (e.g., a clone library), wherein the relative concentration of the cell-free nucleic acid copies consist of the same relative concentration and the same characteristics as found in the biological sample.

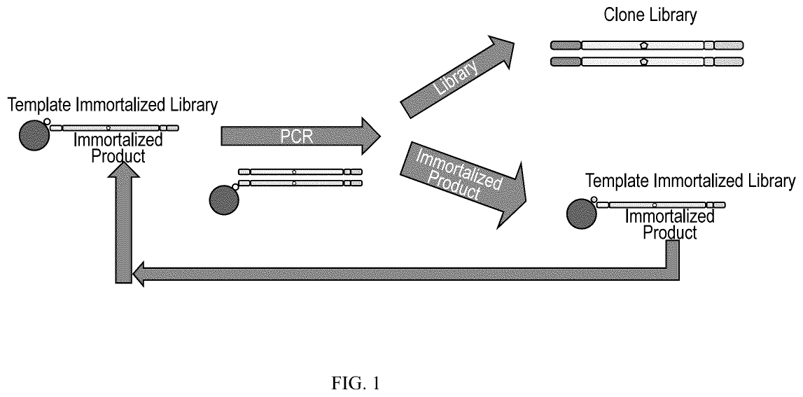

An exemplary method of producing a clone library is shown in FIG. 1. A template nucleic acid from an immortalized library (“immortalized product”) corresponds to the sequence of the nucleic acid in a sample from a subject. The nucleic acid in the template library is attached to a solid surface by a ligand attached at of the nucleic acid termini. The nucleic acid in the template library is copied, e.g., by PCR amplification. The nucleic acid template is recovered (lower right) and the copy is used to produce a clone library (upper right) which can be used, for example, in a diagnostic assay.

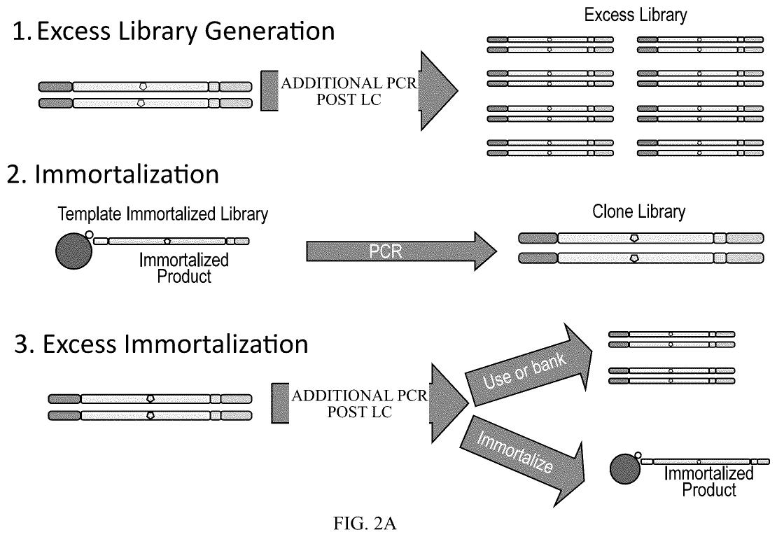

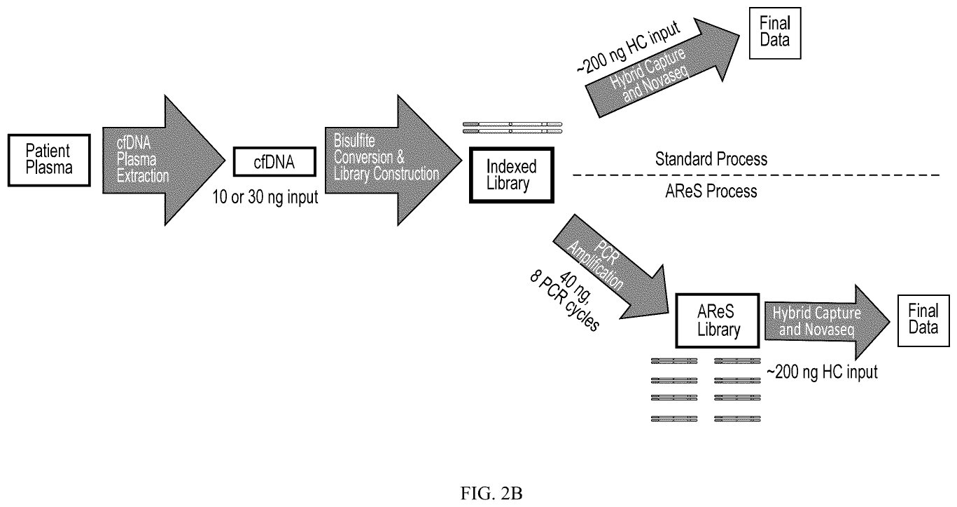

Another exemplary method of producing a clone library is shown in FIG. 2B. A template nucleic acid (cfDNA) is subjected to bisulfite conversion and an indexed library is constructed. The indexed library is then amplified (e.g., by PCR) to form excess library (“AReS Library” also referred to as an immortalized library). The AReS Library can be subjected to hybrid capture to enrich for sequences of interest, and then sequencing (e.g., Novaseq) reactions can be performed to analyze the content of the library.

### I. Definitions

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of 20%, +10%, +5%, or +1% of a given value. The term “about” or “approximately” can mean within an order of magnitude, within 5-fold, or within 2-fold, of a value. Where a particular value is described in the application and claims, unless otherwise stated the term “about” meaning within an acceptable error range for the particular value can be assumed. The term “about” can have the meaning as commonly understood by one of ordinary skill in the art. The term “about” can refer to ±10%. The term “about” can refer to +5%.

As used herein, the term “biological sample,” or “sample” refers to any sample taken from a subject, which can reflect a biological state associated with the subject, and that includes cell free DNA. A biological sample can take any of a variety of forms, such as a liquid biopsy (e.g., blood, urine, stool, saliva, or mucous), or a tissue biopsy, or other solid biopsy. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

As used herein, the terms “nucleic acid” and “nucleic acid molecule” are used interchangeably. The terms refer to nucleic acids of any composition form, such as deoxyribonucleic acid (DNA, e.g., complementary DNA (cDNA), genomic DNA (gDNA) and the like), and/or DNA analogs (e.g., containing base analogs, sugar analogs and/or a non-native backbone and the like), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures. Nucleic acids can comprise protein (e.g., histones, DNA binding proteins, and the like). Nucleic acids analyzed by processes described herein can be substantially isolated and are not substantially associated with protein or other molecules. Nucleic acids can also include derivatives, variants and analogs of DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides can include deoxyadenosine, deoxycytidine, deoxyguanosine and deoxythymidine. A nucleic acid may be prepared using a nucleic acid obtained from a subject as a template.

As used herein, the terms “template nucleic acid” and “template nucleic acid molecule(s)” are used interchangeably. The terms refer to nucleic acid that has been obtained from a sample and processed to form an immortalized library. The template nucleic acid can be nucleic acid obtained directly from the sample, or nucleic acid that is derived from that obtained directly from the sample. Examples of nucleic acid derived from a sample include DNA that has been reverse-transcribed from RNA obtained directly from a sample, or DNA that has be amplified from DNA obtained directly from a sample, for example, by PCR.

As used herein, the term “cell-free nucleic acids” refers to nucleic acid molecules that can be found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject. Cell-free nucleic acids originate from one or more healthy cells and/or from one or more cancer cells, or from non-human sources such bacteria, fungi, viruses. Examples of the cell-free nucleic acids include but are not limited to cell-free DNA (“cfDNA”), including mitochondrial DNA or genomic DNA, and cell-free RNA. In certain embodiments herein, instruments for assessing the quality of the cell-free nucleic acids, such as the TapeStation System from Agilent Technologies (Santa Clara, CA) can be used. Concentrating low-abundance cfDNA can be accomplished, for example using a Qubit Fluorometer from Thermofisher Scientific (Waltham, MA).

As used herein, the term “methylation” refers to a modification of a nucleic acid where a hydrogen atom on the pyrimidine ring of a cytosine base is converted to a methyl group, forming 5-methylcytosine. Methylation can occur at dinucleotides of cytosine and guanine referred to herein as “CpG sites”. Methylation of cytosine can occur in cytosines in other sequence contexts, for example, 5′-CHG-3′ and 5′-CHH-3′, where H is adenine, cytosine or thymine. Cytosine methylation can also be in the form of 5-hydroxymethylcytosine. Methylation of DNA can include methylation of non-cytosine nucleotides, such as N6-methyladenine. Anomalous cfDNA methylation can be identified as hypermethylation or hypomethylation, both of which may be indicative of cancer status. As is well known in the art, DNA methylation anomalies (compared to healthy controls) can cause different effects, which may contribute to cancer.

As used herein the term “methylation index” for each genomic site (e.g., a CpG site, a region of DNA where a cytosine nucleotide is followed by a guanine nucleotide in the linear sequence of bases along its 5′→3′ direction) can refer to the proportion of sequence reads showing methylation at the site over the total number of reads covering that site. The “methylation density” of a region can be the number of reads at sites within a region showing methylation divided by the total number of reads covering the sites in the region. The sites can have specific characteristics, (e.g., the sites can be CpG sites). The “CpG methylation density” of a region can be the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of unconverted cytosines (which can correspond to methylated cytosine) at CpG sites as a proportion of all CpG sites covered by sequence reads mapped to the 100-kb region. In some embodiments, this analysis is performed for other bin sizes, e.g., 50-kb or 1-Mb, etc. In some embodiments, a region is an entire genome or a chromosome or part of a chromosome (e.g., a chromosomal arm). A methylation index of a CpG site can be the same as the methylation density for a region when the region includes that CpG site. The “proportion of methylated cytosines” can refer the number of cytosine sites, “C's,” that are shown to be methylated (for example unconverted after bisulfite conversion) over the total number of analyzed cytosine residues, e.g., including cytosines outside of the CpG context, in the region. The methylation index, methylation density and proportion of methylated cytosines are examples of “methylation levels.”

Certain portions of a genome comprise regions with a high frequency of CpG sites. A CpG site is portion of a genome that has cytosine and guanine separated by only one phosphate group and is often denoted as “5′-C-phosphate-G-3′”, or “CpG” for short. Regions with a high frequency of CpG sites are commonly referred to as “CG islands” or “CGIs”. It has been found that certain CGIs and certain features of certain CGIs in tumor cells tend to be different from the same CGIs or features of the CGIs in healthy cells. Herein, such CGIS and features of the genome are referred to herein as “cancer informative CGIs”, which is defined and described in more detail below. An “informative CpG” can be specified by reference to a specific CpG site, or to a collection of one or more CpG sites by reference to a CG island that contains the collection. These cancer informative CGIs tend to have methylation patterns in tumor cells that are different from the methylation patterns in healthy cells. DNA fragments from other CGIs may not express such differences.

As used herein, the term “methylation profile” (also called methylation status) can include information related to DNA methylation for a region. Information related to DNA methylation can include a methylation index of a CpG site, a methylation density of CpG sites in a region, a distribution of CpG sites over a contiguous region, a pattern or level of methylation for each individual CpG site within a region that contains more than one CpG site, and non-CpG methylation. A methylation profile of a substantial part of the genome can be considered equivalent to the methylome. “DNA methylation” in mammalian genomes can refer to the addition of a methyl group to position 5 of the heterocyclic ring of cytosine (e.g., to produce 5-methylcytosine) among CpG dinucleotides. Methylation of cytosine can occur in cytosines in other sequence contexts, for example, 5′-CHG-3′ and 5′-CHH-3′, where H is adenine, cytosine or thymine. Cytosine methylation can also be in the form of 5-hydroxymethylcytosine. Methylation of DNA can include methylation of non-cytosine nucleotides, such as N6-methyladenine.

As used herein, the term “amplifying” means performing an amplification reaction. In one aspect, an amplification reaction is “template-driven” in that base pairing of reactants, either nucleotides or oligonucleotides, have complements in a template polynucleotide that are required for the creation of reaction products. In one aspect, template-driven reactions are primer extensions with a nucleic acid polymerase, or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references, each of which are incorporated herein by reference herein in their entirety: Mullis et al, U.S. Pat. Nos. 4,683,195; 4,965,188; 4,683,202; 4,800,159 (PCR); Gelfand et al, U.S. Pat. No. 5,210,015 (real-time PCR with “tagman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, the amplification reaction is PCR. An amplification reaction may be a “real-time” amplification if a detection chemistry is available that permits a reaction product to be measured as the amplification reaction progresses, e.g., “real-time PCR”, or “real-time NASBA” as described in Leone et al, Nucleic Acids Research, 26: 2150-2155 (1998), and like references.

A “reaction mixture” means a solution containing all the necessary reactants for performing a reaction, which may include, but is not be limited to, buffering agents to maintain pH at a selected level during a reaction, salts, co-factors, scavengers, and the like.

The terms “fragment” or “segment”, as used interchangeably herein, refer to a portion of a larger polynucleotide molecule. A polynucleotide, for example, can be broken up, or fragmented into, a plurality of segments. Various methods of fragmenting nucleic acid are well known in the art. These methods may be, for example, either chemical or physical or enzymatic in nature. Enzymatic fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave a polynucleotide at known or unknown locations. Physical fragmentation methods may involve subjecting a polynucleotide to a high shear rate. High shear rates may be produced, for example, by moving DNA through a chamber or channel with pits or spikes, or forcing a DNA sample through a restricted size flow passage, e.g., an aperture having a cross sectional dimension in the micron or submicron range. Other physical methods include sonication and nebulization. Combinations of physical and chemical fragmentation methods may likewise be employed, such as fragmentation by heat and ion-mediated hydrolysis. See, e.g., Sambrook et al., “Molecular Cloning: A Laboratory Manual,” 3rd Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range.

The terms “polymerase chain reaction” or “PCR”, as used interchangeably herein, mean a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors that are well-known to those of ordinary skill in the art, e.g., exemplified by the following references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature>90° C., primers annealed at a temperature in the range 50-75° C., and primers extended at a temperature in the range 72-78° C. The term “PCR” encompasses derivative forms of the reaction, including, but not limited to, RT-PCR, real-time PCR, nested PCR, quantitative PCR, multiplexed PCR, and the like. The particular format of PCR being employed is discernible by one skilled in the art from the context of an application. Reaction volumes can range from a few hundred nanoliters, e.g., 200 nL, to a few hundred μL, e.g., 200 μL. “Reverse transcription PCR,” or “RT-PCR,” means a PCR that is preceded by a reverse transcription reaction that converts a target RNA to a complementary single stranded DNA, which is then amplified, an example of which is described in Tecott et al, U.S. Pat. No. 5,168,038, the disclosure of which is incorporated herein by reference in its entirety. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al, U.S. Pat. No. 5,210,015 (“tagman”); Wittwer et al, U.S. Pat. Nos. 6,174,670 and 6,569,627 (intercalating dyes); Tyagi et al, U.S. Pat. No. 5,925,517 (molecular beacons); the disclosures of which are hereby incorporated by reference herein in their entireties. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Asymmetric PCR” means a PCR wherein one of the two primers employed is in great excess concentration so that the reaction is primarily a linear amplification in which one of the two strands of a target nucleic acid is preferentially copied. The excess concentration of asymmetric PCR primers may be expressed as a concentration ratio. Typical ratios are in the range of from 10 to 100. “Multiplexed PCR” means a PCR wherein multiple target sequences (or a single target sequence and one or more reference sequences) are simultaneously carried out in the same reaction mixture, e.g., Bernard et al, Anal. Biochem., 273: 221-228 (1999) (two-color real-time PCR). Usually, distinct sets of primers are employed for each sequence being amplified. Typically, the number of target sequences in a multiplex PCR is in the range of from 2 to 50, or from 2 to 40, or from 2 to 30. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences or internal standards that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β2-microglobulin, ribosomal RNA, and the like. Techniques for quantitative PCR are well-known to those of ordinary skill in the art, as exemplified in the following references, which are incorporated by reference herein in their entireties: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); and Becker-Andre et al, Nucleic Acids Research, 17: 9437-9446 (1989).

The term “primer” as used herein means an oligonucleotide, either natural or synthetic, that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually, primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following reference that is incorporated by reference herein in its entirety: Dieffenbach, editor, PCR Primer: A Laboratory Manual, 2nd Edition (Cold Spring Harbor Press, New York, 2003).

The terms “unique identifier”, “unique sequence tag”, “sequence tag”, “tag” or “barcode”, as used interchangeably herein, refer to an oligonucleotide that is attached to a polynucleotide or template molecule and is used to identify and/or track the polynucleotide or template in a reaction or a series of reactions. A unique identifier may be attached to the 3′- or 5′-end of a polynucleotide or template, or it may be inserted into the interior of such polynucleotide or template to form a linear conjugate, sometimes referred to herein as a “tagged polynucleotide,” or “tagged template,” or the like. A unique identifier may vary widely in size and compositions; the following references, which are incorporated herein by reference in their entireties, provide guidance for selecting sets of unique identifiers appropriate for particular embodiments: Brenner, U.S. Pat. No. 5,635,400; Brenner and Macevicz, U.S. Pat. No. 7,537,897; Brenner et al, Proc. Natl. Acad. Sci., 97: 1665-1670 (2000); Church et al, European patent publication 0 303 459; Shoemaker et al, Nature Genetics, 14: 450-456 (1996); Morris et al, European patent publication 0799897A1; Wallace, U.S. Pat. No. 5,981,179; and the like. Lengths and compositions of unique identifiers can vary widely, and the selection of particular lengths and/or compositions depends on several factors including, without limitation, how unique identifiers are used to generate a readout, e.g., via a hybridization reaction or via an enzymatic reaction, such as sequencing; whether they are labeled, e.g., with a fluorescent dye or the like; the number of distinguishable oligonucleotide identifiers required to unambiguously identify a set of polynucleotides, and the like, and how different the identifiers of a particular set must be in order to ensure reliable identification, e.g., freedom from cross hybridization or misidentification from sequencing errors. In one aspect, unique identifiers can each have a length within a range of from about 2 to about 36 nucleotides, or from about 4 to about 30 nucleotides, or from about 8 to about 20 nucleotides, or from about 6 to about 10 nucleotides. In one aspect, sets of unique identifiers are used, wherein each unique identifiers of a set has a unique nucleotide sequence that differs from that of every other tag of the same set by at least two bases; in another aspect, sets of unique identifiers are used wherein the sequence of each unique identifiers of a set differs from that of every other unique identifiers of the same set by at least three bases.

Aspects of the invention involve the use of unique identifiers. Unique identifiers in accordance with embodiments of the invention can serve many functions. For example, unique sequence tags can include molecular barcode sequences, unique molecular identifier (UMI) sequences, or index sequences. In one embodiment, unique sequence tags (e.g., barcode or index sequences) can be used to identify DNA sequences originating from a common source such as a sample type, tissue, subject, or individual. In accordance with one embodiment, barcodes or index sequences can be used for multiplex sequencing. In one embodiment, unique sequence tags (e.g., unique molecular identifiers (UMIs)) can be used to identify unique nucleic acid sequences from a mixed nucleic acid sample. For example, differing unique molecular identifiers (e.g., UMIs) can be used to differentiate ssDNA molecules, dsDNA molecules, or damaged molecules (e.g., nicked dsDNA) contained in a cfDNA sample. In another embodiment, unique molecular identifiers (e.g., UMIs) can be used to reduce amplification bias, which is the asymmetric amplification of different targets due to differences in nucleic acid composition (e.g., high GC content). The unique molecular identifiers (UMIs) can be used to discriminate between nucleic acid mutations that arise during amplification. The unique sequence tags can be present in a multi-functional nucleic acid adapter, which adapter can comprise both a unique sequence tag and a universal priming site. In some embodiments, unique sequence tags can be greater than about 5, 6, 7, 8, 9, 10, 11, 12, 13, 14, 15, 16, 17, or 18 nucleic acids in length.

In one embodiment, ssDNA molecules in a mixture of dsDNA and ssDNA molecules can be tagged with a unique sequence tags (e.g., ssDNA-specific tags, barcodes or UMIs) using an ssDNA ligation protocol and converted to dsDNA prior to preparation of a combined cfDNA library.

In another embodiment, dsDNA molecules in a mixture of dsDNA and ssDNA molecules can be tagged with unique molecular identifiers (e.g., UMIs) in a dsDNA ligation protocol using Y-shaped sequencing adapters and then ssDNA molecules can be tagged with a unique identifiers (e.g., barcode or unique UMI) and converted to dsDNA.

In some embodiments, the methods of the invention involve differential tagging of populations of cfDNA molecules (e.g., dsDNA molecules, ssDNA molecules, and nicked dsDNA molecules) in a sample with unique sequence tags to distinguish sequence information derived from one population of cfDNA molecules (e.g., dsDNA molecules) from sequence information derived from another population of cfDNA molecules (e.g., ssDNA molecules). Analysis of all populations of cfDNA molecules (e.g., dsDNA molecules, ssDNA molecules, and nicked dsDNA molecules) may increase the sensitivity of certain protocols, for example, a cancer screening protocol. Without being bound by theory, it is believed that ssDNA molecules and/or nicked dsDNA may provide additional valuable insight for cancer detection and screening from a cfDNA sample, and/or may be more representative of tumor content in a cfDNA sample.

In one embodiment, ssDNA molecules in a mixture of dsDNA and ssDNA molecules can be tagged with a unique sequence tags (e.g., ssDNA-specific tags, barcodes or UMIs) using an ssDNA ligation protocol and converted to dsDNA prior to preparation of a combined cfDNA library.

In another embodiment, dsDNA molecules in a mixture of dsDNA and ssDNA molecules can be tagged with unique sequence tags (e.g., UMIs) in a dsDNA ligation protocol using Y-shaped sequencing adapters (also referred to herein as “Y adapters”) and then ssDNA molecules can be tagged with a unique sequence tags (e.g., barcode or unique UMI) and converted to dsDNA.

In one embodiment, the incorporated unique sequences tags and ssDNA-specific tag can be used to distinguish sequencing reads as being originally derived from dsDNA or ssDNA in a cfDNA sample.

In another embodiment, the incorporated unique sequences tags (e.g., UMIs) and ssDNA-specific tags (e.g., barcodes or UMIs) can be used to obtain fragment size information and genome position associated with sequencing reads from nicked dsDNA fragments in a cfDNA sample.

In yet another embodiment, the incorporated unique sequences tags (e.g., UMIs) and ssDNA-specific tags (e.g., barcodes or UMIs) are used to reduce error introduced by amplification, library preparation, and/or sequencing.

As used herein, the term “sensitivity” refers to the ability of a diagnostic assay to correctly identify subjects with a condition of interest. As used herein, the term “specificity” refers to the ability of a diagnostic assay to correctly identify subjects without a condition of interest.

As used herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a women or a child).

### II. Immortalized Libraries

The disclosure relates in part to an immortalized library of template nucleic acid molecules, also referred to herein as archived reference samples (AReS). An immortalized library can include a plurality of nucleic acid molecules from a subject, which can be processed, stored (archived) and retrieved for amplification. In certain embodiments, following an amplification reaction, the immortalized library can be recovered, stored, and can be amplified multiple times without depleting the collection (see, e.g., FIG. 1). In certain embodiments, an immortalized library can include an excess of an amplified library (e.g., an original library), where in the library (e.g., the original library) was constructed from a plurality of nucleic acid molecules from the subject and subjected to multiple cycles of an amplification reaction (e.g., PCR) to produce the excess of library (see, e.g., FIG. 2B).

The template nucleic acid molecules are obtained from a biological sample, and can be, for example, single stranded or double stranded DNA (genomic or mitochondrial) or RNA. In certain embodiments, the template nucleic acids are cell-free nucleic acids such as cell-free DNA or cell-free RNA. In certain embodiments, the template nucleic acid molecules are not taken from a biological sample directly, but are derived from nucleic acid molecules obtained from the biological sample. For example, RNA from a biological sample can be reverse-transcribed, and the resulting DNA molecules can be used to prepare an immortalized library of template DNA.

The template nucleic acid molecules can be subjected to other manipulations, such as one or more enrichment steps. For example, the template nucleic acid molecules can be subjected to hybrid capture to enrich for regions of interest prior to the preparation of the immortalized library. For sequence-specific enrichment, such as hybrid capture, only sequences of interest are captured and amplified to form (1) an original library which is then amplified to produce an immortalized library or (2) an immortalized library that binds or is capable of binding to a solid support. Use of enrichment for regions of interest (targets or target regions) allows quantification of the template nucleic acid targeted for enrichment during a later detection step (e.g., during sequencing of the immortalized library), because only targeted sequence and no non-target sequences would be enriched.

In certain embodiments, recovery of an immortalized library requires a component and/or means for recovering the template nucleic acid. Accordingly, in certain embodiments, the template nucleic acid includes a component such as a solid support or a moiety that binds to a solid support. In certain embodiments, the solid support is selected from a bead (e.g., a paramagnetic bead), a slide, a membrane, a planar surface, a microtiter plate, a filter, a test strip, a slide, a cover slip, and a test tube. In certain embodiments, the moiety that binds to a solid support comprises biotin or avidin/streptavidin, which allows for the recovery of the immortalized library by binding the template nucleic acid to avidin/streptavidin or biotin, respectively. Other examples of moieties that bind a solid support include amine groups that can be bound to a solid surface comprising carboxylic acid by an EDC mediated reaction, and phosphate groups that can be bound to a solid surface (e.g., bead) comprising a carboxy group. The immortalized library can be stored attached the solid surface, or to the moiety that binds to a solid surface, for example, in a storage buffer. A clone library can be amplified, and the immortalized library is then recovered, for example, separated from the clone library, optionally purified, and stored.

In certain embodiments, the solid support or the moiety that binds to a solid support is present on an adapter that is attached to the template nucleic acid during the preparation of the immortalized library. In certain embodiments, each of the plurality of nucleic acid molecules is attached to a solid support via a linker, such as a TEG linker.

In certain embodiments, the template nucleic acid stores information relating to the nucleic acid sequence of all or a portion of the nucleic acid (e.g., cell-free DNA) from a sample (e.g., the presence or absence of a nucleotide polymorphism, indel, sequence rearrangement, mutational frequency, etc.), the copy number of one or more particular nucleotide sequences within the genome (e.g., copy number, allele frequency fractions, single chromosome or entire genome ploidy, etc.), the epigenetic status of all or a portion of the genome (e.g., covalent nucleic acid modifications such as methylation, histone modifications, nucleosome positioning, etc.), the expression profile of the organism's genome (e.g., gene expression levels, isotype expression levels, gene expression ratios, etc.).

In embodiments in which it is useful to record the methylation status of nucleic acids from a sample, the cytosine residues of the template have been selectively deaminated. Selective deamination refers to a process in which cytosine residues are selectively deaminated over 5-methylcytosine residues. Deamination of cytosine forms uracil, effectively inducing a C to T point mutation to allow for detection of methylated cytosines. Methods of deaminating cytosine are known in the art, and include bisulfite conversion and enzymatic conversion. Bisulfite conversion can be performed using commercially available technologies, such as Zymo Gold available from Zymo Research (Irvine, CA) or EpiTect Fast available from Qiagen (Germantown, MD). In certain embodiments, the enzymatic conversion comprises subjecting the nucleic acid to TET2, which oxidizes methylated cytosines, thereby protecting them, and subsequent exposure to APOBEC, which converts unprotected (unmethylated) cytosines to uracils.

Immortalized libraries can comprise an adaptor, which is a portion of nucleic acid added to a nucleic acid obtained from a sample, for example, at its 3′ or 5′ end. The adapter can provide information about the nucleic acid obtained from the sample, provide sequences used for a sequencing reaction or for amplifying the library, and can provide a means for recovering the immortalized library. The adaptor can be compatible with a DNA sequencer to be used and enable the DNA sequencer to detect the DNA fragment for sequencing. Adaptors can be added using commercially available technologies, such as the Accel-NGS Methyl-Seq DNA Library prep technology from Swift Biosciences (Ann Arbor, MI).

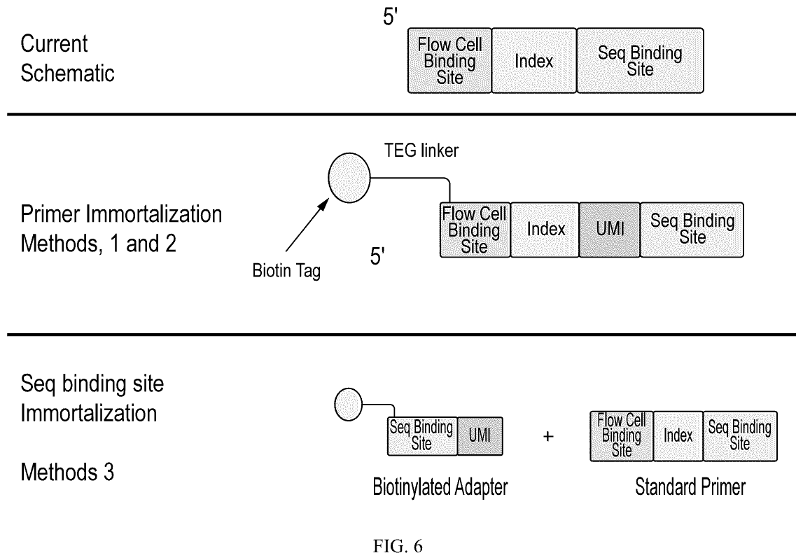

In certain embodiments, the adapter comprises one or more of a flow cell binding site, an index, a unique molecular identifier (UMI), and a sequencing binding site. FIG. 6 shows a schematic diagram showing exemplary adapters suitable for use with the immortalized libraries provided herein. For example, the middle portion of the figure shows an adapter suitable for certain embodiments of the immortalized libraries described herein. In this embodiment, the adapter includes a flow cell binding site, an index sequence, a UMI and a sequence binding site, and the adapter is linked to a biotin tag via a TEG linker. The flow cell binding site allows the nucleic acid molecule to attach to a flow cell for a next generation sequencing reaction. Index sequences, sometimes referred to as 15 and i7, act as barcodes to allow multiplexing of samples. The sequence binding site is used to initiate sequencing. the portions of the adapter described above, and, in addition, includes a UMI (unique molecular identifier) as described herein. Further, the adapter is attached to a biotin tag via a TEG linker. The lower portion of the figure shows a biotinylated adapter and a standard primer suitable for use in the third method described in FIG. 2. In this embodiment, a biotinylated adapter comprising a sequence binding site and a UMI and a standard primer comprising a flow cell binding site, an index and a sequence binding site are used to generate an immortalized library.

In certain embodiments, immortalized libraries are not bound to a solid surface, but instead, a nucleic acid sample (e.g., a liquid biopsy) is processed to form a library as described herein (e.g., selectively deaminated, adapters added), and the library is amplified (e.g., with 5, 6, 7, 8, 9, 10, 11, 12, 13, 14, or 15 cycles) to provide an excess of library. The excess of library can then be used in aliquots to generate clone libraries repeatedly over time in the clinical diagnostic and clinical trials described herein.

The immortalized library may be assessed for certain primary quality control metrics. For example, in certain embodiments, the immortalized library has at least 100× unique coverage, such as between 101× and about 150× coverage, between about 101× and about 200× coverage, between about 101× and about 500× coverage, between about 101× and about 150× coverage, between about 110× and about 200× coverage, between about 110× and about 500× coverage, between about 150× and about 200× coverage, between about 150× and about 500× coverage, or between about 2000× and about 500× coverage.

Another metric for assessing the quality of a library is the fraction of unique reads, or “fraction unique,” which refers to the fraction of reads that are unique out of the total number of reads (e.g., 10M reads). In certain embodiments, the immortalized library has a fraction of unique reads of at least about 0.1, at least about 0.2, at least about 0.3, at least about 0.4, or at least about 0.5 at 10M reads. In certain embodiments, additional sequencing of the library can generate additional unique reads. Accordingly, where an immortalized library does not have a high fraction of unique reads, a library can be sequenced additional times to increase the fraction of unique reads.

In certain embodiments, the immortalized library contains a sufficient conversion efficiency to ensure that there was sufficient methylation to assess biological variation about noise, where conversion efficiency can be measure by determining cytosine cytosine percentage (% CC).

The immortalized library may also be assessed for secondary quality control metrics, including a percentage (%) of pass filter (PF) unique reads of at least 25%, an on target rate of at least 10%, a sequencing alignment rate of at least 50%, total reads of more than about 36 million, and a library size (sum of total unique reads) of at least about 7 million.

In certain embodiments, libraries not meeting one or more of these quality control measurements may be excluded from analysis.

### III. Methods of Making Immortalized Libraries

In another aspect, the disclosure relates to a method of making an immortalized nucleic acid library, comprising obtaining a plurality of fragments of nucleic acid or nucleic acid derived from the nucleic acid from one or more human subjects; attaching an adapter to the nucleic acid or the nucleic acid derived from nucleic acid (for example, a DNA generated from an RNA from a human subject); wherein the adapter comprises a component or a means for recovering the immortalized nucleic acid library.

The component for recovering the immortalized nucleic acid library, if used, can be, for example, a solid support or can be a moiety capable of binding to a solid support. The solid support or the moiety capable of binding to a solid support may be present on an adapter of the template nucleic acid. If the adapter is not bound to a solid support, the method may further comprises binding the adapter to the solid support. In certain embodiments, the solid support is selected from a bead, a slide, a membrane, a planar surface, a microtiter plate, a filter, a test strip, a slide, a cover slip, and a test tube. In certain embodiments, the moiety capable of binding to a solid support is selected from biotin and streptavidin. Other examples of moieties that bind a solid support include amine groups that can be bound to a solid surface comprising carboxylic acid by an EDC mediated reaction, and phosphate groups that can be bound to a solid surface (e.g., bead) comprising a carboxy group.

The nucleic acid used to make the immortalized library can be cell-free nucleic acid, e.g., cell-free DNA. The method of making the immortalized library can include repairing ends of the nucleic acid or the nucleic acid derived from the nucleic acid prior to attaching the adapter, for example, by blunt end repair.

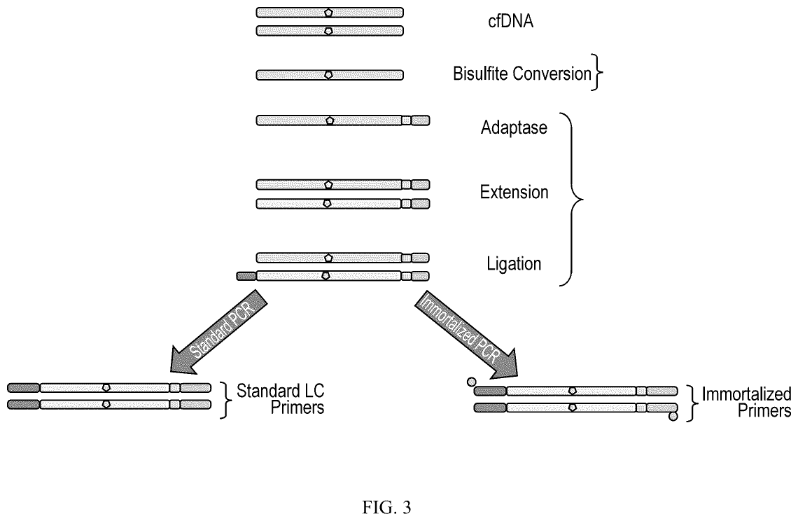

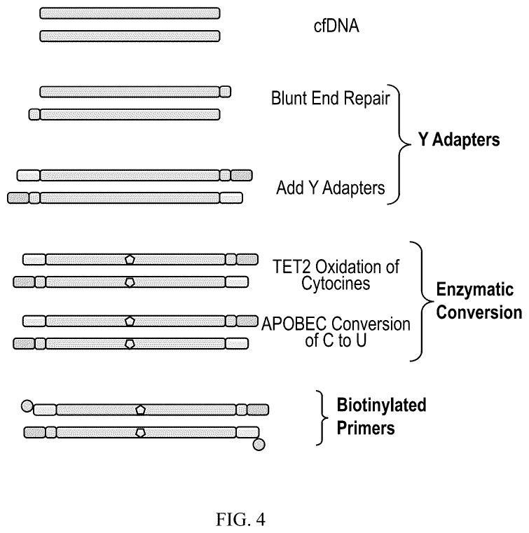

When methylation status of nucleic acid is of interest, e.g., for evaluation in a diagnostic assay, the method can further comprises selectively deaminating cytosine residues of the nucleic acid. In certain embodiments, the step of selectively deaminating cytosine residues is performed prior to attaching the adapter to the nucleic acid, as shown in FIG. 3. In certain embodiments, the step of selectively deaminating cytosine residues is performed after attaching the adapter, for example, as shown in FIG. 4. In certain embodiments, the step of selectively deaminating cytosine residues is performed after attaching the nucleic acid to the solid support.

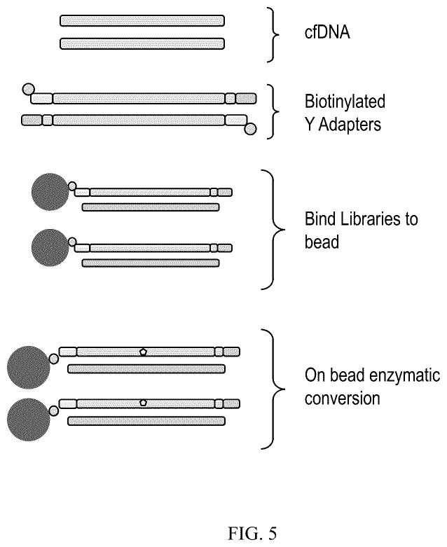

Methods and chemistries for selectively deaminating cytosine residues are known in the art and can include, for example, a bisulfite conversion step. In certain embodiments, the bisulfite conversion step is performed prior to attaching the adapter, as shown in FIG. 3. Other known methods for selectively deaminating cytosine residues include enzymatic conversion. In certain embodiments, enzymatic conversion is performed by TET2 oxidation of cytosines followed by APOBEC conversion of cytosines. In certain embodiments, the enzymatic conversion step is performed after attaching an adapter (e.g., a Y adapter), as shown in FIG. 4. In certain embodiments, the enzymatic conversion step is performed after binding the adapter to a solid support, as shown in FIG. 5 (“on bead enzymatic conversion”).

Adaptors can be attached to a nucleic acid by any means known in the art, for example, as are used in connection with next generation sequencing. For example, adapters, such as a Y adapter, can be attached to a nucleic acid by ligation. In certain embodiments, the adapter is attached by nucleic acid amplification of the cell-free nucleic acid using a primer comprising the adapter. In certain embodiments, the adapter comprises one or more of a flow cell binding site, an index, a unique molecular identifier (UMI), and a sequencing binding site. Such adaptors can be used whether or not the library is attached to a solid support or a moiety capable of binding to a solid support.

In certain embodiments, the solid support or the moiety capable of binding to a solid support is connected to the adapter via a linker. In certain embodiments, the linker is a TEG linker.

Following the production of an initial library (e.g., a cfDNA library) from a nucleic acid sample from a subject, the library can be subject to a nucleic acid amplification reaction (e.g., a PCR amplification reaction) to produce an immortalized library (e.g., an AReS excess library).

Depending upon the intended use for the immortalized library, the immortalized library may require a yield (e.g., as low as 1 ng) sufficient for use in expanded testing models, such as for research and development, quality control, hypothesis testing, clinical utility, etc. In certain embodiments, construction of the immortalized library includes determining amplification reaction conditions needed for sufficient yield, the reaction conditions selected from (1) determining an amount of an initial library for use in the amplification reaction, (2) determining a number of amplification cycles (e.g., PCR cycles) for expanding the nucleic acids, and (3) determining primer concentrations.

The immortalized library can be produced using from about 0.01 ng to about 320 ng of the initial library as input for the PCR reaction, for example, from about 0.01 ng to about 0.1 ng, from about 0.01 ng to about 1 ng, from about 0.01 ng to about 5 ng, from about 0.01 ng to about 10 ng, from about 0.01 ng to about 20 ng, from about 0.01 ng to about 30 ng, from about 0.01 ng to about 40 ng, from about 0.01 ng to about 50 ng, from about 0.01 ng to about 60 ng, from about 0.1 ng to about 1 ng, from about 0.1 ng to about 5 ng, from about 0.1 ng to about 10 ng, from about 0.1 ng to about 20 ng, from about 0.1 ng to about 30 ng, from about 0.1 ng to about 40 ng, from about 0.1 ng to about 50 ng, from about 0.1 ng to about 60 ng, from about 1 ng to about 5 ng, from about 1 ng to about 10 ng, from about 1 ng to about 20 ng, from about 1 ng to about 30 ng, from about 1 ng to about 40 ng, from about 1 ng to about 50 ng, from about 1 ng to about 60 ng, from about 5 ng to about 10 ng, from about 5 ng to about 20 ng, from about 5 ng to about 30 ng, from about 5 ng to about 40 ng, from about 5 ng to about 50 ng, from about 5 ng to about 60 ng, from about 10 ng to about 20 ng, from about 10 ng to about 30 ng, from about 10 ng to about 40 ng, from about 10 ng to about 50 ng, from about 10 ng to about 60 ng, from about 20 ng to about 30 ng, from about 20 ng to about 40 ng, from about 20 ng to about 50 ng, from about 20 ng to about 60 ng, from about 30 ng to about 40 ng, from about 30 ng to about 50 ng, from about 30 ng to about 60 ng, from about 40 ng to about 50 ng, from about 40 ng to about 60 ng, from about 50 ng to about 60 ng of the initial library. In certain embodiments, using at least 0.01 ng of the initial library results in a median target coverage of at least about 100. In certain embodiments, using at least 0.1 ng of the initial library results in a median target coverage of at least about 100. In certain embodiments, using at least 1 ng of the initial library results in a median target coverage of at least about 100.

The nucleic acid amplification reaction is performed for a number of cycles sufficient to produce between about 20 and about 3,000 ng (e.g., between about 26 and about 2,400 ng) of immortalized library. In certain embodiments, the nucleic acid amplification reaction, e.g., a PCR reaction, is performed for about 5 to about 20 cycles, e.g., for 5, 6, 7, 8, 9, 10, 11, 12, 13, 14, 15, 16, 17, 18, 19, or 20 cycles, or any range falling therein. In certain embodiments, the reaction is performed for about 6 to about 10 cycles. In certain embodiments, the reaction is performed for about 8 cycles.

The yield of the nucleic acid amplification reaction can be increased by increasing the concentration of primers used. In certain embodiments, primers are used at a concentration of about 1×, about 2×, about 3× or about 4×, for example, at about 10 μM, about 20 μM, about 30 μM, or about 40 μM. In certain embodiments, the primers are used at a concentration of about 3× or about 30 μM.

In certain embodiments, the method further comprises storing the immortalized library in a storage medium. In certain embodiments, the storage medium preserves the integrity of the immortalized library, wherein the integrity of the immortalized library is measured by maintenance of concordance of methylated variants over at least 6 weeks, at least 6 months, at least 1 year, at least 2 years, at least 5 years, or at least 10 years.

To make downstream testing economically feasible, it may be beneficial for the immortalized library to have a low fraction of PCR duplicates, thereby to have a sufficient unique rate to provide enough informative data for testing. For example, in certain embodiments, the fraction of PCT duplicates is from about 10% to about 70%, for example, from about 10% to about 20%, from about 10% to about 30%, from about 10% to about 40%, from about 10% to about 50%, from about 10% to about 60%, from about 20% to about 30%, from about 20% to about 40%, from about 20% to about 50%, from about 20% to about 60%, from about 20% to about 70%, from about 30% to about 40%, from about 30% to about 50%, from about 30% to about 60%, from about 30% to about 70%, from about 40% to about 50%, from about 40% to about 60%, from about 40% to about 70%, from about 50% to about 60%, from about 50% to about 70%, or from about 60% to about 70%.

### IV. Methods of Using Immortalized Libraries

The immortalized libraries described herein can be amplified to produce a clone library, the clone library can be used in an assay, such as an assay being evaluated in a clinical trial, and the immortalized library can be stored and used again later in the same or a different assay. Thus, in certain aspects, the disclosure relates to a method of amplifying an immortalized nucleic acid library to produce a clone library, the method comprising amplifying the nucleic acid library to produce a clone library of amplified nucleic acid.

In certain embodiments, the method further comprises analyzing a feature of the clone library to generate a classifier score. In certain embodiments, concordance of the classifier score is maintained as compared to an amplification using a non-immortalized nucleic acid library. In certain embodiments, concordance of the methylation variants is maintained over at least 5 amplification reactions, at least 10 amplification reactions, or at least 20 amplification reactions, for example, 5-10 amplification reactions, 5-15 amplification reactions, 5-20 amplification reactions, 10-15 amplification reactions, 10-20 amplification reactions, or 15-20 amplification reactions. In certain embodiments, the concordance is determined by measuring at least one of qPCR, iSeq, and NovaSeq. In certain embodiments, one or more metrics of percentage of methylated GC, coverage, alignment rate, unique rate, and on target rate of the clone library are comparable to an amplification using a non-immortalized nucleic acid library. In certain embodiments, the method further comprises recovering the immortalized library after the amplifying step.

In certain embodiments, the method includes subjecting a clone library or an immortalized library (e.g. an AReS Library) to DNA sequencing by a DNA sequencer. The DNA sequencer outputs what is referred to herein as the “sequencer data”, also described in more detail below. The sequencer data output from the DNA sequencer is processed by data processing system to provide a classification for the individual. For example, the data processing system can determine a likelihood of the individual having a neoplasm such as an early-stage cancerous tumor based on that individual's liquid biopsy. Details of an example implementation of such a data processing system are provided in more detail below.

To assess methylation status from cell-free DNA or cell-free RNA using sequencing, sequencer data from a training set of samples for which a classification, e.g., cancer or tumor yes/no, is known, a set of cancer informative methylation sites (such as CGIs) can be identified and selected from among a larger set of CGIs. Second, metrics related to the methylation patterns within these CGIs can be computed. Such metrics can be used to assist in identifying and selecting cancer informative CGIs. Third, given a set of cancer informative CGIs, samples can be processed to enrich for the cell-free DNA or cell-free RNA fragments containing the cancer informative CGIs. Fourth, clone libraries can be deep sequenced, using next generation sequencers, on the order of several hundred times, so that there is, on average, a large number of cell-free DNA or cell-free RNA fragments sequenced for the cancer informative CGIs, thus increasing the likelihood of sequencing cell-free DNA or cell-free RNA fragments originating from a neoplasm. Fifth, using the sequencer data for the training set, a computational model can be built that can predict likelihood of presence of neoplasm in other individuals based on liquid biopsies from those individuals. In some implementations, such a computational model can be built using various machine learning techniques and classification models. Specifically, a set of features can be computed from sequencer data for the training set, where the features are the combination of one or more metrics for selected cancer informative CGIs, and a model can be trained such that the trained computational model has a sensitivity and specificity suitable to predict a likelihood of presence in the individual of a neoplasm such as an early-stage cancerous solid tumor. Finally, the computational model can be used to process the sequencer data originating from liquid biopsies of other individuals to screen for the likelihood of presence of a neoplasm.

In certain embodiments, the method can include adding (“spiking in”) an amount of a reference nucleic acid to allow for quantifying the amount of an analyte present in a sample. For example, to quantify the reads of unique cfDNA fragments in an immortalized library that originate from cancer cells, the method may include the step of adding (“spiking in”) a known amount of a reference nucleic acid and then quantifying the amount of reference nucleic acid and the amount of nucleic acid (reads) attributable to cancer cells. Knowing the amount of reference nucleic acid in the sample allows for the quantification of nucleic acid (number of reads) attributable to cancer cells.

An example system for obtaining sequencer data from a clone library or an immortalized library (e.g. an AReS Library) will now be described. The example system processes a set of clone libraries or an immortalized library (e.g. an AReS Library), for what is called herein a training set, to obtain sequencer data for the training set, and which processes one or more clone library or an immortalized library (e.g. an AReS Library), for one or more individuals for whom screening is being performed, to obtain sequencer data for the one or more liquid biopsies. The training set comprises a set of samples for which a classification, e.g., cancer or tumor yes/no, is known. Other information about the classification may be known such as a tissue of origin or the part of the body affected by the neoplasm, a stage of development of a cancer or other kind of tumor, a type of cancer or other kind of tumor.

Samples from individual(s), or sample(s) from the training set, are input to the sample processing system(s). Such systems generally use reagents, probes, and other ingredients and processes to process a sample or to obtain a processed sample for sequencing. Generally, but not necessarily, the sample processing system(s) used to process both kinds of samples and are the same.

The clone library or a portion (e.g., an aliquot) of an immortalized library (e.g. an AReS Library) is placed in the DNA sequencer. The DNA sequencer sequences the prepared sample based on control instructions, such as instructions about the depth of sequencing to be performed. The output of the DNA sequencer is the sequencer data.

In certain embodiments, clone or immortalized libraries (e.g. AReS Libraries) are pooled for sample multiplexing. Sample multiplexing, also known as multiplex sequencing, allows large numbers of libraries to be pooled and sequenced simultaneously during a single run on a sequencing instrument. Sample multiplexing is useful when targeting specific genomic regions or working with smaller genomes. Pooling samples exponentially increases the number of samples analyzed in a single run, without drastically increasing cost or time.

With multiplex sequencing, unique identifiers (e.g., individual “barcode” sequences) may be added to each nucleic acid fragment during next-generation sequencing (NGS) library preparation so that each read can be identified and sorted before the final data analysis.

Methods for preparing pooled libraries for NGS are well-established, and include techniques such as barcoding and combinatorial pooled sequencing. Shokralla et al., “Next-generation DNA barcoding: using next-generation sequencing to enhance and accelerate DNA barcode capture from single specimens”, Mol. Ecol Resources (2014)14, 892-901 [5]; Cao and Sun, “Combinatorial pooled sequencing: experiment design and decoding”, Quantitative Biology 2016, 4(1): 36-46 [6].

The nucleic acid in the clone library or the immortalized library (e.g. the AReS Library) then may be enriched for selected cancer informative CGIs. This step may be accomplished using hybrid capture probe sets that enable targeting of selected genomic regions, followed by PCR amplification. Examples of such hybrid capture probe sets include the KAPA HyperPrep Kit and SeqCAP Epi Enrichment System from Roche Diagnostics (Pleasanton, CA). The selected genomic regions may be those cancer informative CGIs selected and used for training a computational model, or selected and used in a trained computational model, as described herein.

The clone or immortalized library (e.g. AReS Library) then is ready for sequencing, which may be accomplished, for example, using a commercially available sequencer such as the Illumina NovaSeq, the output of which is a FASTQ format data file.

Sequencer data, for the purposes described herein, generally includes, for each DNA fragment, data indicating a location in the genome for the fragment, and, for each CpG within the DNA fragment, data indicating whether that CpG is methylated. However, when output by a DNA sequencer into a FASTQ format data file, the raw data can be many gigabytes, e.g., 20 to 30 gigabytes, of data per individual.

Using the location information for each DNA fragment from the sequencer data, data corresponding to the DNA fragments can be grouped by cancer informative CGIs in which the DNA fragments are found. Such segmentation of the sequencer data for each subject into data by cancer informative CGI can be performed for a full set of cancer informative CGIs, resulting in a respective subset of the sequencer data for each cancer informative CGI.

The example implementation is described herein in the context of analyzing cfDNA based on DNA sequencing. Other DNA analysis methods (e.g., PCR) can be used to obtain similar information about methylation state of cfDNA fragments or about the presence of mutations, etc. The term “sequencer data” herein is intended to include any data that provides information about the sequence of a nucleic acid, including methylation of CpGs in selected cancer informative CGIs, regardless of the origin or equipment used to obtain that information.

In certain embodiments, sequencer data is processed by a system as will now be described.

As noted above, the training set comprises a set of samples (e.g., liquid biopsies) for which respective classifications for each sample are known. Examples of known classifications for the samples, typically called “labels”, include information such as a cancer or tumor yes/no, type of cancer, stage of cancer, a tissue of origin or the part of the body affected by a neoplasm, or type of neoplasm. Data called “features” are derived from the sequencer data obtained from the training set. These features are used to build or train a computational model that can classify unknown samples using features computed from the sequencer data for those samples. The sequencer data for the liquid biopsy for the individual for whom screening is being performed is an input, from which features are computed and input into this trained computational model. The trained computational model provides an output indicative of the likelihood of presence of a neoplasm such as an early-stage cancer in the individual. A training set generally includes samples both with and without a neoplasm such as cancer, from multiple stages including early stages of development, and from multiple types of tumors or cancers.

The sequencer data and labels for the training set are processed to compute features to be used by a computational model. The combination of computed features and the labels are used by a training module to train the computational module. Typically, such training is performed by dividing the sets of features computed for different samples in the training set into a train set and a test set, and continually adjusting parameters of the trained computational module using the train set while minimizing errors in classifying the test set.

The feature computation module, for which example implementations are described in more detail below, computes one or more metrics related to the methylation for each of the cancer informative CGIs selected to build the computational model. The specific metrics used and cancer informative CGIs selected can depend on the training set and desired classification. Example selections are provided in more detail below. The selected metric(s) for the selected cancer informative CGIs are computed for the training set to provide the train set and the test set. Generally, the combination of the one or more metrics and the cancer informative CGIs are selected such that, given the training set, the trained computational model has a sensitivity and specificity suitable to predict a likelihood of presence in the individual of a neoplasm, such as an early-stage cancerous tumor. Such selection is described in more detail below.

Given a trained model, that model can be used to process features computed from sequencer data for an individual, unknown or unclassified, sample. A feature computation module, which performs similar computations as module, processes the sequencer data for an individual to compute the one or more metrics for the selected cancer informative CGIs (used in the trained computational model) based the sequencer data to provide feature data. This feature data is input to the trained computational model, which provides a result, a classification, for that individual sample. Note that if this classification is verified as indicated at, the data for the sample could be added to the data for the training set, and can be used to update the trained computational model.

Typically, to train a computational model, a training data set is used. The training data set includes data for examples for which a prediction or label or other outcome is known. To predict the likelihood of presence of a neoplasm, the training data set includes data for examples where there is no tumor or cancer, and where there is a tumor or cancer. As an example, a training set of liquid biopsies (blood) is obtained, of which were known to be from individuals with cancer across different stages (I, II, and III) and are known to be from individuals who were cancer free. Generally, the more samples obtained the better, with a distribution of subjects across different types of neoplasms to be detected and across different stages of development to be detected. The liquid biopsies from such individuals provide a training set, which is processed as described above, and then sequenced to produce corresponding sequencer data for the training set. The sequencer data is then processed to provide a set of computed features for each sample.

As mentioned above, a feature computation module computes one or more metrics for selected cancer informative CGIs based the sequencer data related to that CGI. The combination of metrics and cancer informative CGIs for which they are computed are used to create a set of features based on the sequencer data from a training set, or for a sample to be screened.

Sequencer data is first grouped by individual, and then, for each individual, the data for DNA fragments are grouped by CGI and aligned by CpG. The data representing the methylation information for each DNA fragment within the CGI is reduced by computing one or more of the metrics for the CGI. Thus, the raw sequencer data is reduced for an individual to a collection of the metrics computed for each of the selected cancer informative CGIs. Such data can be represented in a table where each row is a CGI and each column includes a metric computed for that CGI.

The output of the computational model is a form of prediction, indicating a likelihood that the individual from whom a sample was obtained has a neoplasm, such as an early-stage cancerous tumor or other solid tumor present in the body. This prediction can be in the form of a probability between zero and one, or a binary output, such as a yes or no answer, or a score (which may be compared to one or more thresholds), or other format. The output can be accompanied by additional information indicating, for example, a level of confidence in the prediction. The output typically depends on the form of the computational model used.

In some implementations, that output indicates the likelihood of the presence of a neoplasm such as cancer, without indicating a type of tumor or cancer, i.e., the affected tissue. In some implementations, the output of the model can indicate a type of cancer. In some implementations, the output of the model can indicate a type of cancer, and then one or more additional models can be applied to the data to indicate a type of cancer. In some implementations, a separate model for each type of cancer can be used, and an ensembling process can process the outputs of the separate models.

Exemplary cancer informative CGIs are identified in, e.g., U.S. Patent Publication 2020/0109456A1, which is hereby incorporated by reference, specifically the “Table I” of CGIs listed in that published patent application.

In another aspect, the disclosure relates to a method of treating a subject having a condition, the method comprising, administering to the subject a therapeutic agent suitable for treating the condition, wherein the subject was diagnosed with the condition using a method comprising the following steps: (1) obtaining a plurality of fragments of nucleic acid or nucleic acid derived from nucleic acid from the subject; (2) attaching an adapter to the nucleic acid or the nucleic acid derived from the nucleic acid, wherein the adapter is bound to a solid support, to form an immortalized library; (3) amplifying the immortalized library to form a clone library of amplified nucleic acid; (4) assaying the clone library with an assay for a diagnostic parameter to generate an output, wherein the output correlates with presence of the condition in the subject.

In another aspect, the disclosure relates to a method of treating a subject having a condition, the method comprising, administering to the subject a therapeutic agent suitable for treating the condition, wherein the subject was diagnosed with the condition using a method comprising the following steps: (1) obtaining or deriving a plurality of nucleic acids from the subject; (2) producing a library from the nucleic acids; (3) amplifying the library to produce an excess of amplified library; (4) and assaying a portion (e.g., an aliquot) of the excess of amplified library with an assay for a diagnostic parameter to generate an output, wherein the output correlates with presence of the condition in the subject.

A. Use of Immortalized Libraries for Conducting or Emulating Clinical Trials

In one aspect, the disclosure relates to a method of emulating or conducting a human clinical study that uses a plurality of human subjects to evaluate a diagnostic parameter. The method includes providing a clone library of amplified nucleic acid representative of nucleic acid from the plurality of human subjects, wherein the clone library was prepared by a process comprising obtaining the nucleic acid from the plurality of human subjects; attaching the nucleic acid or nucleic acid derived from the nucleic acid to a solid support or other component for recovering the nucleic acid, optionally via an adapter, to form an immortalized library of nucleic acid. The method further comprises amplifying the library of nucleic acid to form the clone library of amplified nucleic acid; assaying the clone library with an assay for the diagnostic parameter to generate an output, wherein the output correlates with presence or absence of a condition in each of the plurality of human subjects; and comparing the output to a reference standard to determine sensitivity or specificity of the diagnostic parameter. In certain embodiments, the nucleic acid of the immortalized library and/or the clone library of each of the plurality of human subjects comprises a unique identifier. In certain embodiments, the unique identifier allows for the identification of the source of the sample, such that the output can be correlated with, for example, whether or not the human subject has a condition, thereby to correlate the diagnostic parameter with the condition (or lack of condition) in the subject.

In another aspect, the disclosure relates to a method of emulating or conducting a human clinical study that uses a plurality of human subjects to evaluate a diagnostic parameter. The method includes providing a clone library of amplified nucleic acid from each of the plurality of human subjects, wherein at least one clone library was prepared by a process comprising obtaining nucleic acid from a human subject; attaching the nucleic acid or nucleic acid derived from the nucleic acid to a solid support or other component for recovering the nucleic acid, optionally via an adapter, to form an immortalized library of template nucleic acid and amplifying the immortalized library of template nucleic acid to form the at least one clone library of amplified nucleic acid. The method further includes assaying the clone libraries with an assay for the diagnostic parameter to generate an output, wherein the output correlates with presence or absence of a condition in each of the plurality of human subjects; and comparing the output to a reference standard to determine sensitivity or specificity of the diagnostic parameter.

In certain embodiments, the condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments neurodegenerative disease is one of Alzheimer's disease, Parkinson's disease, amyotrophic lateral sclerosis (ALS), frontotemporal dementia (FTD).

The diagnostic parameter can be any diagnostic parameter that can be assessed using nucleic acid. For example, the diagnostic parameter may include a methylation state of one or more nucleotides in the nucleic acid or the presence of a mutation in the nucleic acid. In certain embodiments, a diagnostic device is used to evaluate the diagnostic parameter.

The output of the assay can be any detectable signal, such as a one or more fluorescent signals, sequencer data, etc. In certain embodiments, the output is associated with the presence of the condition (e.g., cancer). In certain embodiments, the output is associated with the absence of the condition (e.g., cancer).

In certain embodiments, the method further comprises recovering the immortalized library. Recovering the immortalized library can, in some embodiments, refer to separating the immortalized library from, for example, a clone library, so that the immortalized library can be stored and/or used again in another amplification reaction to produce a second or subsequent clone library.

In certain embodiments, the method further comprises assaying a second diagnostic parameter, comprising the steps of (1) assaying a second clone library with an assay for the second diagnostic parameter to generate a second output, wherein the second clone library was generated by amplifying the immortalized library, wherein the second output correlates with presence or absence of the condition or a second condition in each of the plurality of human subjects; and (2) comparing the second output to a second reference standard to determine sensitivity or specificity of the second diagnostic parameter.

The method can further comprises repeating steps (1) and (2) with at least one additional clone library to determine sensitivity or specificity for at least one additional diagnostic parameter.

In certain embodiments, two or more of the diagnostic parameter, second diagnostic parameter, and at least one additional diagnostic parameter are the same. For example, for a diagnostic device that measures a diagnostic parameter, following an initial clinical trial, the device may be changed and require additional round of testing using the improved device. In this embodiments, one or more of the immortalized libraries used in the first clinical trial can be amplified to produce one or more additional clone libraries, thereby preventing the need to obtain additional samples. Accordingly, the diagnostic parameter measured by the diagnostic device may be tested multiple times over different iterations of the device to determine the sensitivity or specificity of the diagnostic parameter.

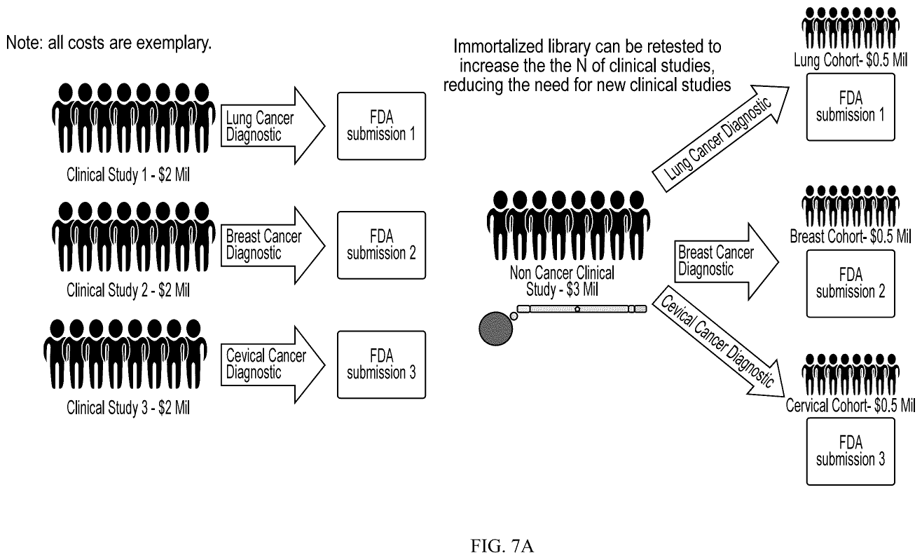

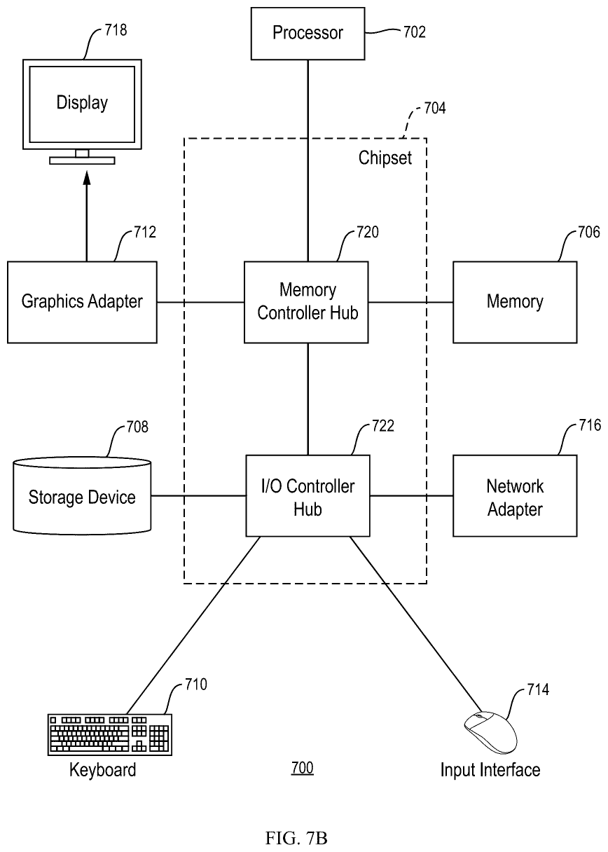

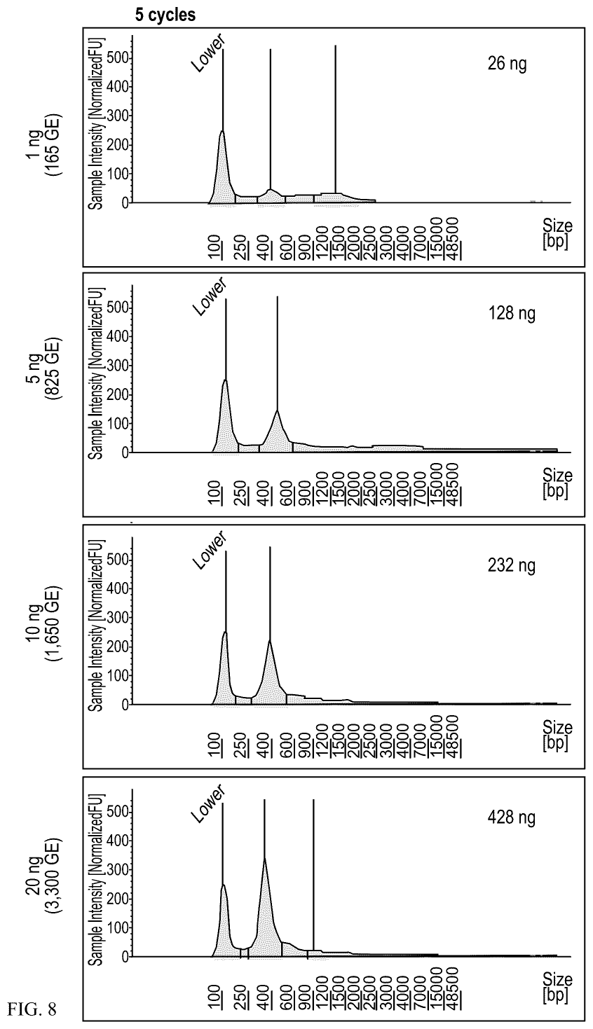

In certain embodiments, the second diagnostic parameter is different from the diagnostic parameter. For example, immortalized libraries from subject not having a condition (e.g., not having cancer), can be used in two or more clinical trials to assay different diagnostic parameters, for example, a lung cancer diagnostic and a breast cancer diagnostic (see, for example, FIG. 7A).

In certain embodiments, the second output is the same as the output. In certain embodiments, the second output is different from the output. In certain embodiments, the output and/or the second output comprises a fluorescent signal, sequencer data, etc. In certain embodiments, the output and/or the second output is associated with the presence of the condition (e.g., cancer). In certain embodiments, the output and/or the second output is associated with the absence of the condition (e.g., cancer).

In certain embodiments, the nucleic acid is cell-free nucleic acid and the second diagnostic parameter or the at least one additional diagnostic parameter comprises a methylation state of one or more nucleotides in the cell-free nucleic acid or the presence of a mutation in the cell-free nucleic acid.

In certain embodiments, the second condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments, the neurodegenerative disease is one of Alzheimer's disease, Parkinson's disease, amyotrophic lateral sclerosis (ALS), frontotemporal dementia (FTD).

The clinical trial can be spaced apart in time by weeks, months, or years. In certain embodiments, the second clone library is generated at least 4 weeks, at least 4 months, at least 6 months, at least 1 year, at least 2 years, at least 5 years, or at least 10 years after the clone library is generated. In certain embodiments, at least one additional clone library is generated at least 4 weeks, at least 4 months, at least 6 months, at least 1 year, at least 2 years, at least 5 years, or at least 10 years after the second clone library is generated.

In one aspect, the disclosure relates to a method of emulating or conducting a human clinical study that uses a plurality of human subjects to evaluate a diagnostic parameter. The method includes providing a portion of an archived reference sample (AReS) library of amplified nucleic acid representative of nucleic acid from the plurality of human subjects, wherein the library was prepared by a process comprising obtaining the nucleic acid from the plurality of human subjects; preparing an original library, and performing an amplification reaction to produce an excess of amplified library, thereby forming the AReS library. The method further comprises assaying the portion of the AReS library with an assay for the diagnostic parameter to generate an output, wherein the output correlates with presence or absence of a condition in each of the plurality of human subjects; and comparing the output to a reference standard to determine sensitivity or specificity of the diagnostic parameter. In certain embodiments, the nucleic acid of the AReS library and/or the original library of each of the plurality of human subjects comprises a unique identifier. In certain embodiments, the unique identifier allows for the identification of the source of the sample, such that the output can be correlated with, for example, whether or not the human subject has a condition, thereby to correlate the diagnostic parameter with the condition (or lack of condition) in the subject.

In another aspect, the disclosure relates to a method of emulating or conducting a human clinical study that uses a plurality of human subjects to evaluate a diagnostic parameter. The method includes providing a portion of an archived reference sample (AReS) library of amplified nucleic acid representative of nucleic acid from each of the plurality of human subjects, wherein at least one portion of an AReS library was prepared by a process comprising obtaining nucleic acid from a human subject; preparing an original library, and performing an amplification reaction to produce an excess of amplified library, thereby forming the AReS library. The method further includes assaying the AReS libraries with an assay for the diagnostic parameter to generate an output, wherein the output correlates with presence or absence of a condition in each of the plurality of human subjects; and comparing the output to a reference standard to determine sensitivity or specificity of the diagnostic parameter.

In certain embodiments, the condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments neurodegenerative disease is one of Alzheimer's disease, Parkinson's disease, amyotrophic lateral sclerosis (ALS), frontotemporal dementia (FTD).

The diagnostic parameter can be any diagnostic parameter that can be assessed using nucleic acid. For example, the diagnostic parameter may include a methylation state of one or more nucleotides in the nucleic acid or the presence of a mutation in the nucleic acid. In certain embodiments, a diagnostic device is used to evaluate the diagnostic parameter.

The output of the assay can be any detectable signal, such as a one or more fluorescent signals, sequencer data, etc. In certain embodiments, the output is associated with the presence of the condition (e.g., cancer). In certain embodiments, the output is associated with the absence of the condition (e.g., cancer).

In certain embodiments, the method further comprises assaying a second diagnostic parameter, comprising the steps of (1) assaying a second portion (e.g., aliquot) of an AReS library with an assay for the second diagnostic parameter to generate a second output, wherein the second output correlates with presence or absence of the condition or a second condition in each of the plurality of human subjects; and (2) comparing the second output to a second reference standard to determine sensitivity or specificity of the second diagnostic parameter.

The method can further comprises repeating steps (1) and (2) with at least one additional portion (e.g., aliquot) of the AReS library to determine sensitivity or specificity for at least one additional diagnostic parameter.

In certain embodiments, two or more of the diagnostic parameter, second diagnostic parameter, and at least one additional diagnostic parameter are the same. For example, for a diagnostic device that measures a diagnostic parameter, following an initial clinical trial, the device may be changed and require additional round of testing using the improved device. In this embodiments, one or more portions (e.g., aliquots) of the AReS libraries used in the first clinical trial can be used, thereby preventing the need to obtain additional samples. Accordingly, the diagnostic parameter measured by the diagnostic device may be tested multiple times over different iterations of the device to determine the sensitivity or specificity of the diagnostic parameter.

In certain embodiments, the second diagnostic parameter is different from the diagnostic parameter. For example, immortalized libraries from subject not having a condition (e.g., not having cancer), can be used in two or more clinical trials to assay different diagnostic parameters, for example, a lung cancer diagnostic and a breast cancer diagnostic (see, for example, FIG. 7A).

In certain embodiments, the second output is the same as the output. In certain embodiments, the second output is different from the output. In certain embodiments, the output and/or the second output comprises a fluorescent signal, sequencer data, etc. In certain embodiments, the output and/or the second output is associated with the presence of the condition (e.g., cancer). In certain embodiments, the output and/or the second output is associated with the absence of the condition (e.g., cancer).

In certain embodiments, the nucleic acid is cell-free nucleic acid and the second diagnostic parameter or the at least one additional diagnostic parameter comprises a methylation state of one or more nucleotides in the cell-free nucleic acid or the presence of a mutation in the cell-free nucleic acid.

In certain embodiments, the second condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments, the neurodegenerative disease is one of Alzheimer's disease, Parkinson's disease, amyotrophic lateral sclerosis (ALS), frontotemporal dementia (FTD).

The clinical trial can be spaced apart in time by weeks, months, or years. In certain embodiments, the second clone library is generated at least 4 weeks, at least 4 months, at least 6 months, at least 1 year, at least 2 years, at least 5 years, or at least 10 years after the clone library is generated. In certain embodiments, at least one additional clone library is generated at least 4 weeks, at least 4 months, at least 6 months, at least 1 year, at least 2 years, at least 5 years, or at least 10 years after the second clone library is generated.

B. Evaluating a Diagnostic Assay

In another aspect, the disclosure relates to a method for determining specificity or sensitivity of a diagnostic assay, comprising providing a clone library of amplified nucleic acid representative of nucleic acid from a plurality of human subjects, wherein the clone library was prepared by a process comprising obtaining the nucleic acid from the plurality of human subjects; attaching the nucleic acid or nucleic acid derived from the nucleic acid to a solid support, optionally via an adapter, to form an immortalized library of nucleic acid; and amplifying the library of nucleic acid to form the clone library of amplified nucleic acid; assaying the clone library with the diagnostic assay to generate an output, wherein the output correlates with presence or absence of a condition in each of the plurality of human subjects; and comparing the output to a reference standard to determine sensitivity or specificity of the diagnostic assay. In certain embodiments, the nucleic acid of the immortalized library and/or the clone library of each of the plurality of human subjects comprises a unique identifier. In certain embodiments, the unique identifier allows for the identification of the source of the sample, such that the output can be correlated with, for example, whether or not the human subject has a condition, thereby to correlate the diagnostic parameter with the condition (or lack of condition) in the subject.

In another aspect, the disclosure relates to a method for determining specificity or sensitivity of a diagnostic assay, comprising providing a clone library of amplified nucleic acid from each of a plurality of human subjects, wherein at least one clone library was prepared by a process comprising obtaining nucleic acid from a human subject; attaching the nucleic acid or nucleic acid derived from the nucleic acid to a solid support, optionally via an adapter, to form an immortalized library of template nucleic acid; and amplifying the immortalized library of template nucleic acid to form the at least one clone library of amplified nucleic acid; assaying the clone libraries with the diagnostic assay to generate an output, wherein the output correlates with presence or absence of a condition in each of the plurality of human subjects; and comparing the output to a reference standard to determine sensitivity or specificity of the diagnostic assay.

The output can be any signal (e.g., a fluorescent signal or sequencer data), and can correlate with the presence or absence of any condition useful for diagnosis, including, for example, cancer, inflammatory disease, neurodegenerative disease, autoimmune disorder, neuromuscular disease, metabolic disorder, cardiac disease, or fibrotic disease, or a risk of developing any one of the foregoing. In certain embodiments neurodegenerative disease is one of Alzheimer's disease, Parkinson's disease, amyotrophic lateral sclerosis (ALS), frontotemporal dementia (FTD).

The diagnostic parameter can be any diagnostic parameter that can be assessed using nucleic acid. For example, the diagnostic parameter may include a methylation state of one or more nucleotides in the nucleic acid or the presence of a mutation in the nucleic acid. In certain embodiments, a diagnostic device is used to evaluate the diagnostic parameter.

The method further can further comprise recovering the immortalized library, for example, by washing the solid support and resuspending it in a suitable buffer, and storing it for further use.

Additional clone libraries can be amplified from the immortalized library to evaluate the same or different diagnostic parameters. In certain embodiments, the method includes assaying a second diagnostic parameter, comprising the steps of (1) assaying a second clone library with an assay for the second diagnostic parameter to generate a second output, wherein the second clone library was generated by amplifying the immortalized library, wherein the second output correlates with presence or absence of the condition or a second condition in each of the plurality of human subjects; and (2) comparing the second output to a second reference standard to determine sensitivity or specificity of the second diagnostic parameter.

Steps (1) and (2) can be repeated with at least one additional clone library to determine sensitivity or specificity for at least one additional diagnostic parameter.

In certain embodiments, two or more of the diagnostic parameter, second diagnostic parameter, and at least one additional diagnostic parameter are the same. For example, for a diagnostic device that measures a diagnostic parameter, following an initial assay, the device may be changed and require one or more additional rounds of testing using the improved device. In this embodiments, one or more of the immortalized libraries used in the first assay can be amplified to produce one or more additional clone libraries, thereby preventing the need to obtain additional samples. This approach may have the additional advantage of more consistent results, due to the use of identical or nearly identical samples. Accordingly, the diagnostic parameter measured by the diagnostic device may be tested multiple times over different iterations of the device to determine the sensitivity or specificity of the diagnostic parameter.

In certain embodiments, the second diagnostic parameter is different from the diagnostic parameter. In one example, immortalized libraries from subject not having a condition (e.g., not having cancer), can be used in two or more assays to test different diagnostic parameters, for example, a lung cancer diagnostic and a breast cancer diagnostic (see, for example, FIG. 7A).

The second output can be the same as the output or different from the output, for example, if a different diagnostic parameter is being used. In certain embodiments, the output and/or the second output comprises a fluorescent signal or sequencer data.

The nucleic acid used to generate the immortalized library can be cell-free nucleic acid and the second diagnostic parameter or the at least one additional diagnostic parameter can include a methylation state of one or more nucleotides in the cell-free nucleic acid or the presence of a mutation in the cell-free nucleic acid.

In certain embodiments, the second condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments, the neurodegenerative disease is one of Alzheimer's disease, Parkinson's disease, amyotrophic lateral sclerosis (ALS), frontotemporal dementia (FTD).

Because the immortalized assay can be recovered and stored in between assays, the assays can be performed weeks, months, or years apart. In certain embodiments, the second clone library is generated at least 4 weeks, at least 4 months, at least 6 months, at least 1 year, at least 2 years, at least 5 years, or at least 10 years after the clone library is generated. In certain embodiments, at least one additional clone library is generated at least 4 weeks, at least 4 months, at least 6 months, at least 1 year, at least 2 years, at least 5 years, or at least 10 years after the second clone library is generated. In certain embodiments, the second clone library is generated between 4 weeks and 4 months, between 4 months and 6 months, between 6 months and 1 year, between 1 year and 2 years, between 1 year and 5 years or between 1 year and 10 years after the clone library is generated. In certain embodiments, at least one additional clone library is generated between 4 weeks and 4 months, between 4 months and 6 months, between 6 months and 1 year, between 1 year and 2 years, between 1 year and 5 years or between 1 year and 10 years after the second clone library is generated.

In another aspect, the disclosure relates to a method of assessing the correlation of a potential diagnostic parameter with a condition in a plurality of human subjects, comprising providing a clone library of amplified nucleic acid from each of the plurality of human subjects, wherein the clone library was prepared by a process comprising obtaining nucleic acid from the plurality of human subjects; attaching the nucleic acid or nucleic acid derived from the nucleic acid to a solid support, optionally via an adapter, to form an immortalized library of template nucleic acid; and amplifying the library of template nucleic acid to form the clone library of amplified nucleic acid; assaying the clone library with an assay for the diagnostic parameter to generate an output; determining whether the output correlates with presence or absence of a condition in each of the plurality of human subjects.

In another aspect, the disclosure relates to a method of assessing the correlation of a potential diagnostic parameter with a condition in a plurality of human subjects, comprising providing a clone library of amplified nucleic representative of nucleic acid from the plurality of human subjects, wherein the clone library was prepared by a process comprising obtaining nucleic acid from the plurality of human subjects attaching the nucleic acid or nucleic acid derived from the nucleic acid to a solid support, optionally via an adapter, to form an immortalized library of template nucleic acid; and amplifying the library of template nucleic acid to form the clone library of amplified nucleic acid; assaying the clone library with an assay for the diagnostic parameter to generate an output; determining whether the output correlates with presence or absence of a condition in each of the plurality of human subjects. In certain embodiments, the nucleic acid of the immortalized library and/or the clone library of each of the plurality of human subjects comprises a unique identifier.

The output can be any signal (e.g., a fluorescent signal or sequencer data), and can correlate with the presence or absence of any condition useful for diagnosis, including, for example, cancer, inflammatory disease, neurodegenerative disease, autoimmune disorder, neuromuscular disease, metabolic disorder, cardiac disease, or fibrotic disease, or a risk of developing any one of the foregoing. In certain embodiments neurodegenerative disease is one of Alzheimer's disease, Parkinson's disease, amyotrophic lateral sclerosis (ALS), frontotemporal dementia (FTD).

In certain embodiments, the nucleic acid comprises cell-free nucleic acid.

In certain embodiments, the diagnostic parameter comprises a methylation state of one or more nucleotides in the cell-free nucleic acid or the presence of a mutation in the cell-free nucleic acid.

In certain embodiments, a diagnostic device is used to evaluate the diagnostic parameter.

The method further can further comprise recovering the immortalized library, for example, by washing the solid support and resuspending it in a suitable buffer, and storing it for further use.

In certain embodiments, the method further comprises assaying a second diagnostic parameter, comprising the steps of (1) assaying a second clone library with an assay for the second diagnostic parameter to generate a second output, wherein the second clone library was generated by amplifying the immortalized library, determining whether the output correlates with presence or absence of the condition or a second condition in each of the plurality of human subjects. In certain embodiments, the method further comprises repeating steps (1) and (2) with at least one additional clone library to determine whether the output correlates with presence or absence of the condition, the second condition, or at least one addition condition for at least one additional diagnostic parameter. In certain embodiments, the second diagnostic parameter is the same as the diagnostic parameter. In certain embodiments, the second diagnostic parameter is different from the diagnostic parameter. In certain embodiments, the at least one additional diagnostic parameter is the same as the diagnostic parameter and the second diagnostic parameter. In certain embodiments, the second output is the same as the output. In certain embodiments, the second output is different from the output. In certain embodiments, the nucleic acid comprises cell-free nucleic acid and the second diagnostic parameter or the at least one additional diagnostic parameter comprises a methylation state of one or more nucleotides in the cell-free nucleic acid or the presence of a mutation in the cell-free nucleic acid. In certain embodiments, the second output comprises a fluorescent signal.

In certain embodiments, the second condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments, the neurodegenerative disease is one of Alzheimer's disease, Parkinson's disease, amyotrophic lateral sclerosis (ALS), frontotemporal dementia (FTD).

In certain embodiments, the second clone library is generated at least 4 weeks, at least 4 months, at least 6 months, at least 1 year, at least 2 years, at least 5 years, or at least 10 years after the clone library is generated. In certain embodiments, the at least one additional clone library is generated at least 4 weeks, at least 4 months, at least 6 months, at least 1 year, at least 2 years, at least 5 years, or at least 10 years after the second clone library is generated.

In another aspect, the disclosure relates to a method for determining specificity or sensitivity of a diagnostic assay, comprising providing a clone library of amplified nucleic acid representative of nucleic acid from a plurality of human subjects, wherein the clone library was prepared by a process comprising obtaining the nucleic acid from the plurality of human subjects; forming a immortalized library of template nucleic acid, wherein the immortalized library comprises a property allowing for the recovery of the template nucleic acid; and amplifying the library of nucleic acid to form the clone library of amplified nucleic acid; assaying the clone library with the diagnostic assay to generate an output, wherein the output correlates with presence or absence of a condition in each of the plurality of human subjects; and comparing the output to a reference standard to determine sensitivity or specificity of the diagnostic assay.

The property allowing for the recovery of the template nucleic acid can include a solid support or a moiety that binds to a solid support, as described elsewhere herein. In certain embodiments, the property allowing for the recovery of the template nucleic acid comprises a detectable (e.g., fluorescent) moiety.

In certain embodiments, the nucleic acid of the immortalized library and/or the clone library of each of the plurality of human subjects comprises a unique identifier.

In certain embodiments, the nucleic acid is attached to the solid support via the adapter. In certain embodiments, the adapter comprises a moiety that binds to the solid support. In certain embodiments, the adapter comprises the solid support.

In certain embodiments, the method further comprises selectively deaminating cytosine residues. In certain embodiments, the step of selectively deaminating cytosine residues is performed prior to attaching the adapter. In certain embodiments, the step of selectively deaminating cytosine residues is performed after attaching the adapter. In certain embodiments, the step of selectively deaminating cytosine residues is performed after attaching the nucleic acid to the solid support. In certain embodiments, the step of selectively deaminating cytosine residues comprises a bisulfite conversion step. In certain embodiments, the bisulfite conversion step is performed prior to attaching the adapter. In certain embodiments, the step of selectively deaminating cytosine residues comprises an enzymatic conversion step. In certain embodiments, the enzymatic conversion step is selected from TET2 oxidation of cytosines and APOBEC conversion of cytosines. In certain embodiments, the enzymatic conversion step is performed after attaching the adapter. In certain embodiments, the enzymatic conversion step is performed after attaching the nucleic acid to the solid support.

In certain embodiments, the adapter is attached by ligation. In certain embodiments, the adapter is attached by nucleic acid amplification of the nucleic acid using a primer comprising the adapter. In certain embodiments, the solid support is selected from a bead, a slide, a membrane, a planar surface, a microtiter plate, a filter, a test strip, a slide, a cover slip, and a test tube. In certain embodiments, the moiety that binds to the solid support is selected from biotin and streptavidin. In certain embodiments, the solid support or the moiety that binds to the solid support is connected to the adapter via a linker. In certain embodiments, the linker is a TEG linker. In certain embodiments, the adapter comprises one or more of a flow cell binding site, an index, a unique molecular identifier (UMI), and a sequencing binding site.

In certain embodiments, the condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments, the neurodegenerative disease is one of Alzheimer's disease, Parkinson's disease, amyotrophic lateral sclerosis (ALS), frontotemporal dementia (FTD).

In certain embodiments, the nucleic acid comprises cell-free nucleic acid and the diagnostic parameter comprises a methylation state of one or more nucleotides in the cell-free nucleic acid or the presence of a mutation in the cell-free nucleic acid.

In certain embodiments, the output comprises a fluorescent signal.

The diagnostic parameter can be any diagnostic parameter that can be assessed using nucleic acid. For example, the diagnostic parameter may include a methylation state of one or more nucleotides in the nucleic acid or the presence of a mutation in the nucleic acid. In certain embodiments, a diagnostic device is used to evaluate the diagnostic parameter.

The method further can further comprise recovering the immortalized library, for example, by washing the solid support and resuspending it in a suitable buffer, and storing it for further use.

In certain embodiments, the method further comprises assaying a second diagnostic parameter, comprising the steps of (1) assaying a second clone library with an assay for the second diagnostic parameter to generate a second output, wherein the second clone library was generated by amplifying the immortalized library, determining whether the output correlates with presence or absence of the condition or a second condition in each of the plurality of human subjects. In certain embodiments, the method further comprises repeating steps (1) and (2) with at least one additional clone library to determine whether the output correlates with presence or absence of the condition, the second condition, or at least one addition condition for at least one additional diagnostic parameter. In certain embodiments, the second diagnostic parameter is the same as the diagnostic parameter. In certain embodiments, the second diagnostic parameter is different from the diagnostic parameter. In certain embodiments, the at least one additional diagnostic parameter is the same as the diagnostic parameter and the second diagnostic parameter. In certain embodiments, the second output is the same as the output. In certain embodiments, the second output is different from the output. In certain embodiments, the nucleic acid comprises cell-free nucleic acid and the second diagnostic parameter or the at least one additional diagnostic parameter comprises a methylation state of one or more nucleotides in the cell-free nucleic acid or the presence of a mutation in the cell-free nucleic acid. In certain embodiments, the second output comprises a fluorescent signal.

In certain embodiments, the second condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments, the neurodegenerative disease is one of Alzheimer's disease, Parkinson's disease, amyotrophic lateral sclerosis (ALS), frontotemporal dementia (FTD).

In certain embodiments, the second clone library is generated at least 4 weeks, at least 4 months, at least 6 months, at least 1 year, at least 2 years, at least 5 years, or at least 10 years after the clone library is generated. In certain embodiments, the at least one additional clone library is generated at least 4 weeks, at least 4 months, at least 6 months, at least 1 year, at least 2 years, at least 5 years, or at least 10 years after the second clone library is generated.

In another aspect, the disclosure relates to a method for simultaneously analyzing a bio-response in nucleic acid from a plurality of human subjects, comprising providing a clone library of amplified nucleic acid representative of nucleic acid from a plurality of human subjects, wherein the clone library was prepared by a process comprising obtaining nucleic acid from the plurality of human subjects; attaching the nucleic acid or nucleic acid derived from the nucleic acid to a solid support, optionally via an adapter, to form an immortalized library of template nucleic acid; and amplifying the immortalized library of template nucleic acid to form the clone library of amplified nucleic acid; assaying the clone library for the bio-response, wherein the bio-response is indicative of a clinical outcome assessment.

A bio-response can be a biological or biochemical response, e.g., CpG methylation that occurs as a results of a biological process occurring in a human subject. In certain embodiments, the bio-response is indicative of a clinical outcome assessment, such as a risk of developing or the development of an early stage of a condition. For example, a bio-response may include methylation status at one or more cancer informative CGIs.

In another aspect, the disclosure relates to a method for determining specificity or sensitivity of a diagnostic assay. The method includes providing a portion of an archived reference sample (AReS) library of amplified nucleic acid representative of nucleic acid from a plurality of human subjects, wherein at least one AReS library was prepared by a process comprising obtaining nucleic acid from a human subject; preparing an original library, and performing an amplification reaction to produce an excess of amplified library, thereby forming the AReS library. The method further includes assaying the portion of the AReS library with the diagnostic assay to generate an output, wherein the output correlates with presence or absence of a condition in each of the plurality of human subjects; and comparing the output to a reference standard to determine sensitivity or specificity of the diagnostic assay. In certain embodiments, the nucleic acid of the AReS library of the original library of each of the plurality of human subjects comprises a unique identifier. In certain embodiments, the unique identifier allows for the identification of the source of the sample, such that the output can be correlated with, for example, whether or not the human subject has a condition, thereby to correlate the diagnostic parameter with the condition (or lack of condition) in the subject.

In another aspect, the disclosure relates to a method for determining specificity or sensitivity of a diagnostic assay, comprising providing a portion of an archived reference sample (AReS) library from each of a plurality of human subjects, wherein at least one clone library was prepared by a process comprising obtaining nucleic acid from a human subject; preparing an original library, and performing an amplification reaction to produce an excess of amplified library, thereby forming the AReS library. The method further includes assaying the portions of the AReS libraries with the diagnostic assay to generate an output, wherein the output correlates with presence or absence of a condition in each of the plurality of human subjects; and comparing the output to a reference standard to determine sensitivity or specificity of the diagnostic assay.

The output can be any signal (e.g., a fluorescent signal or sequencer data), and can correlate with the presence or absence of any condition useful for diagnosis, including, for example, cancer, inflammatory disease, neurodegenerative disease, autoimmune disorder, neuromuscular disease, metabolic disorder, cardiac disease, or fibrotic disease, or a risk of developing any one of the foregoing. In certain embodiments neurodegenerative disease is one of Alzheimer's disease, Parkinson's disease, amyotrophic lateral sclerosis (ALS), frontotemporal dementia (FTD).

The diagnostic parameter can be any diagnostic parameter that can be assessed using nucleic acid. For example, the diagnostic parameter may include a methylation state of one or more nucleotides in the nucleic acid or the presence of a mutation in the nucleic acid. In certain embodiments, a diagnostic device is used to evaluate the diagnostic parameter.

The method further can further comprise recovering the immortalized library, for example, by washing the solid support and resuspending it in a suitable buffer, and storing it for further use.

Additional portions (e.g., aliquots) of the libraries can be amplified from the immortalized library to evaluate the same or different diagnostic parameters. In certain embodiments, the method includes assaying a second diagnostic parameter, comprising the steps of (1) assaying a second portion of the AReS library with an assay for the second diagnostic parameter to generate a second output, wherein the second output correlates with presence or absence of the condition or a second condition in each of the plurality of human subjects; and (2) comparing the second output to a second reference standard to determine sensitivity or specificity of the second diagnostic parameter.

Steps (1) and (2) can be repeated with at least one additional portion (e.g., aliquot) of the AReS library to determine sensitivity or specificity for at least one additional diagnostic parameter.

In certain embodiments, two or more of the diagnostic parameter, second diagnostic parameter, and at least one additional diagnostic parameter are the same. For example, for a diagnostic device that measures a diagnostic parameter, following an initial assay, the device may be changed and require one or more additional rounds of testing using the improved device. In this embodiments, one or more portions (e.g., aliquots) of the AReS libraries used in the first assay can be used, thereby preventing the need to obtain additional samples. This approach may have the additional advantage of more consistent results, due to the use of identical or nearly identical samples. Accordingly, the diagnostic parameter measured by the diagnostic device may be tested multiple times over different iterations of the device to determine the sensitivity or specificity of the diagnostic parameter.

In certain embodiments, the second diagnostic parameter is different from the diagnostic parameter. In one example, immortalized libraries from subject not having a condition (e.g., not having cancer), can be used in two or more assays to test different diagnostic parameters, for example, a lung cancer diagnostic and a breast cancer diagnostic (see, for example, FIG. 7A).

The second output can be the same as the output or different from the output, for example, if a different diagnostic parameter is being used. In certain embodiments, the output and/or the second output comprises a fluorescent signal or sequencer data.

The nucleic acid used to generate the immortalized library can be cell-free nucleic acid and the second diagnostic parameter or the at least one additional diagnostic parameter can include a methylation state of one or more nucleotides in the cell-free nucleic acid or the presence of a mutation in the cell-free nucleic acid.

In certain embodiments, the second condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments, the neurodegenerative disease is one of Alzheimer's disease, Parkinson's disease, amyotrophic lateral sclerosis (ALS), frontotemporal dementia (FTD).

Because the AReS library can be stored in between assays, the assays can be performed weeks, months, or years apart. In certain embodiments, the second clone library is generated at least 4 weeks, at least 4 months, at least 6 months, at least 1 year, at least 2 years, at least 5 years, or at least 10 years after the clone library is generated. In certain embodiments, at least one additional clone library is generated at least 4 weeks, at least 4 months, at least 6 months, at least 1 year, at least 2 years, at least 5 years, or at least 10 years after the second clone library is generated. In certain embodiments, the second clone library is generated between 4 weeks and 4 months, between 4 months and 6 months, between 6 months and 1 year, between 1 year and 2 years, between 1 year and 5 years or between 1 year and 10 years after the clone library is generated. In certain embodiments, at least one additional clone library is generated between 4 weeks and 4 months, between 4 months and 6 months, between 6 months and 1 year, between 1 year and 2 years, between 1 year and 5 years or between 1 year and 10 years after the second clone library is generated.

In another aspect, the disclosure relates to a method of assessing the correlation of a potential diagnostic parameter with a condition in a plurality of human subjects, comprising providing a portion of an AReS library comprising amplified nucleic acid from each of the plurality of human subjects, wherein the wherein at least one AReS library was prepared by a process comprising obtaining nucleic acid from a human subject; preparing an original library, and performing an amplification reaction to produce an excess of amplified library, thereby forming the AReS library. The method includes assaying the portion of the AReS library with an assay for the diagnostic parameter to generate an output and determining whether the output correlates with presence or absence of a condition in each of the plurality of human subjects.

In another aspect, the disclosure relates to a method of assessing the correlation of a potential diagnostic parameter with a condition in a plurality of human subjects, comprising providing a portion of an AReS library of amplified nucleic representative of nucleic acid from the plurality of human subjects, wherein the AReS library was prepared by a process comprising obtaining nucleic acid from a human subject; preparing an original library, and performing an amplification reaction to produce an excess of amplified library, thereby forming the AReS library. The method includes assaying the portion of the AReS library with an assay for the diagnostic parameter to generate an output; determining whether the output correlates with presence or absence of a condition in each of the plurality of human subjects. In certain embodiments, the nucleic acid of the AReS library and/or the original library of each of the plurality of human subjects comprises a unique identifier.

The output can be any signal (e.g., a fluorescent signal or sequencer data), and can correlate with the presence or absence of any condition useful for diagnosis, including, for example, cancer, inflammatory disease, neurodegenerative disease, autoimmune disorder, neuromuscular disease, metabolic disorder, cardiac disease, or fibrotic disease, or a risk of developing any one of the foregoing. In certain embodiments neurodegenerative disease is one of Alzheimer's disease, Parkinson's disease, amyotrophic lateral sclerosis (ALS), frontotemporal dementia (FTD).

In certain embodiments, the nucleic acid comprises cell-free nucleic acid.

In certain embodiments, the diagnostic parameter comprises a methylation state of one or more nucleotides in the cell-free nucleic acid or the presence of a mutation in the cell-free nucleic acid.

In certain embodiments, a diagnostic device is used to evaluate the diagnostic parameter.

The method further can further comprise recovering the immortalized library, for example, by washing the solid support and resuspending it in a suitable buffer, and storing it for further use.

In certain embodiments, the method further comprises assaying a second diagnostic parameter, comprising the steps of (1) assaying a second portion (e.g., aliquot) of the AReS library with an assay for the second diagnostic parameter to generate a second output, determining whether the output correlates with presence or absence of the condition or a second condition in each of the plurality of human subjects. In certain embodiments, the method further comprises repeating steps (1) and (2) with at least one additional portion of the AReS library to determine whether the output correlates with presence or absence of the condition, the second condition, or at least one addition condition for at least one additional diagnostic parameter. In certain embodiments, the second diagnostic parameter is the same as the diagnostic parameter. In certain embodiments, the second diagnostic parameter is different from the diagnostic parameter. In certain embodiments, the at least one additional diagnostic parameter is the same as the diagnostic parameter and the second diagnostic parameter. In certain embodiments, the second output is the same as the output. In certain embodiments, the second output is different from the output. In certain embodiments, the nucleic acid comprises cell-free nucleic acid and the second diagnostic parameter or the at least one additional diagnostic parameter comprises a methylation state of one or more nucleotides in the cell-free nucleic acid or the presence of a mutation in the cell-free nucleic acid. In certain embodiments, the second output comprises a fluorescent signal.

In certain embodiments, the second condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments, the neurodegenerative disease is one of Alzheimer's disease, Parkinson's disease, amyotrophic lateral sclerosis (ALS), frontotemporal dementia (FTD).

In certain embodiments, the second portion of the AReS library is obtained at least 4 weeks, at least 4 months, at least 6 months, at least 1 year, at least 2 years, at least 5 years, or at least 10 years after the AReS library is created. In certain embodiments, the at least one additional portion of the AReS library is created at least 4 weeks, at least 4 months, at least 6 months, at least 1 year, at least 2 years, at least 5 years, or at least 10 years after the AReS library is created.

In another aspect, the disclosure relates to a method for determining specificity or sensitivity of a diagnostic assay, comprising providing a portion of an AReS library of amplified nucleic acid representative of nucleic acid from a plurality of human subjects, wherein the AReS library was prepared by a process comprising obtaining nucleic acid from a human subject; preparing an original library, and performing an amplification reaction to produce an excess of amplified library, thereby forming the AReS library. The method further comprises assaying the portion of the AReS library with the diagnostic assay to generate an output, wherein the output correlates with presence or absence of a condition in each of the plurality of human subjects; and comparing the output to a reference standard to determine sensitivity or specificity of the diagnostic assay.

In certain embodiments, the nucleic acid of the AReS library and/or the original library of each of the plurality of human subjects comprises a unique identifier.

In certain embodiments, the method further comprises selectively deaminating cytosine residues. In certain embodiments, the step of selectively deaminating cytosine residues comprises a bisulfite conversion step. In certain embodiments, the step of selectively deaminating cytosine residues comprises an enzymatic conversion step. In certain embodiments, the enzymatic conversion step is selected from TET2 oxidation of cytosines and APOBEC conversion of cytosines.

In certain embodiments, the nucleic acids of the original library comprise one or more of a flow cell binding site, an index, a unique molecular identifier (UMI), and a sequencing binding site.

In certain embodiments, the condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments, the neurodegenerative disease is one of Alzheimer's disease, Parkinson's disease, amyotrophic lateral sclerosis (ALS), frontotemporal dementia (FTD).

In certain embodiments, the nucleic acid comprises cell-free nucleic acid and the diagnostic parameter comprises a methylation state of one or more nucleotides in the cell-free nucleic acid or the presence of a mutation in the cell-free nucleic acid.

In certain embodiments, the output comprises a fluorescent signal.

The diagnostic parameter can be any diagnostic parameter that can be assessed using nucleic acid. For example, the diagnostic parameter may include a methylation state of one or more nucleotides in the nucleic acid or the presence of a mutation in the nucleic acid. In certain embodiments, a diagnostic device is used to evaluate the diagnostic parameter.

In certain embodiments, the method further comprises assaying a second diagnostic parameter, comprising the steps of (1) assaying a second portion (e.g., aliquot) of the AReS library with an assay for the second diagnostic parameter to generate a second output, determining whether the output correlates with presence or absence of the condition or a second condition in each of the plurality of human subjects. In certain embodiments, the method further comprises repeating steps (1) and (2) with at least one additional portion (e.g., aliquot) of the AReS library to determine whether the output correlates with presence or absence of the condition, the second condition, or at least one addition condition for at least one additional diagnostic parameter. In certain embodiments, the second diagnostic parameter is the same as the diagnostic parameter. In certain embodiments, the second diagnostic parameter is different from the diagnostic parameter. In certain embodiments, the at least one additional diagnostic parameter is the same as the diagnostic parameter and the second diagnostic parameter. In certain embodiments, the second output is the same as the output. In certain embodiments, the second output is different from the output. In certain embodiments, the nucleic acid comprises cell-free nucleic acid and the second diagnostic parameter or the at least one additional diagnostic parameter comprises a methylation state of one or more nucleotides in the cell-free nucleic acid or the presence of a mutation in the cell-free nucleic acid. In certain embodiments, the second output comprises a fluorescent signal.

In certain embodiments, the second condition is selected from cancer, inflammatory disease, neurodegenerative disease, autoimmune disorder, neuromuscular disease, metabolic disorder, cardiac disease, or fibrotic disease, or a risk of developing any one of the foregoing. In certain embodiments, the neurodegenerative disease is one of Alzheimer's disease, Parkinson's disease, amyotrophic lateral sclerosis (ALS), frontotemporal dementia (FTD).

In certain embodiments, the second portion (e.g., aliquot) of the AReS library is obtained at least 4 weeks, at least 4 months, at least 6 months, at least 1 year, at least 2 years, at least 5 years, or at least 10 years after the AReS library is generated. In certain embodiments, the at least one additional portion (e.g., aliquot) of the AReS library is generated at least 4 weeks, at least 4 months, at least 6 months, at least 1 year, at least 2 years, at least 5 years, or at least 10 years after the AReS library is generated.

In another aspect, the disclosure relates to a method for simultaneously analyzing a bio-response in nucleic acid from a plurality of human subjects, comprising providing a portion of an AReS library of amplified nucleic acid representative of nucleic acid from a plurality of human subjects, wherein the AReS library was prepared by a process comprising obtaining nucleic acid from a human subject; preparing an original library, and performing an amplification reaction to produce an excess of amplified library, thereby forming the AReS library; assaying the portion of the AReS library for the bio-response, wherein the bio-response is indicative of a clinical outcome assessment.

A bio-response can be a biological or biochemical response, e.g., CpG methylation that occurs as a results of a biological process occurring in a human subject. In certain embodiments, the bio-response is indicative of a clinical outcome assessment, such as a risk of developing or the development of an early stage of a condition. For example, a bio-response may include methylation status at one or more cancer informative CGIs.

### Systems for Sequencing and Analyzing a Nucleic Acid

In another aspect, the disclosure relates to a system for simultaneously analyzing a bio-response in nucleic acid from a plurality of human subjects, the system comprising (a) a processor; (b) a data storage comprising sequence information derived from one or more clone libraries obtained from the plurality of human subjects, wherein the one or more clone library was prepared by a process comprising obtaining nucleic acid from the plurality of human subjects; attaching the nucleic acid or nucleic acid derived from the nucleic acid to a solid support, optionally via an adapter, to form an immortalized library of template nucleic acid; and amplifying the immortalized library of template nucleic acid to form the clone library of amplified nucleic acid; (c) a non-transitory computer readable medium comprising instructions that, when executed by the processor, cause the processor to: analyze the sequence information for the bio-response, wherein the bio-response is indicative of a clinical outcome assessment.

A bio-response can be a biological or biochemical response, e.g., CpG methylation that occurs as a results of a biological process occurring in a human subject. In certain embodiments, the bio-response is indicative of a clinical outcome assessment, such as a risk of developing or the development of an early stage of a condition. For example, a bio-response may include methylation status at one or more cancer informative CGIs.

Systems disclosed herein are useful for sequencing and analyzing a nucleic acid. In various embodiments, such a system can include one or more sets of reagents for amplifying template nucleic acids of an immortalized library to form a clone library and/or for performing an assay, an apparatus configured to amplify the template nucleic acids of the immortalized library and/or perform an assay on the clone library, and a computer system communicatively coupled to the apparatus to obtain sequence information derived from the clone library and analyze the sequence information for the bio-response.

The one or more sets of reagents enable amplification of template nucleic acids of an immortalized library. For example, reagents can include a set of primers that, when combined with the template nucleic acids of the immortalized library, enables amplification of the template nucleic acids and results in generation of the clone library. As another example, reagents can be primers or read sequences that are incorporated for sequencing of the clone library. Therefore, sequence information can include read sequences (e.g., R1 or R2 read sequences).

The apparatus is configured to determine the sequence information. In various embodiments, the apparatus can be configured to perform a nucleic acid amplification assay (e.g., polymerase chain reaction assay) on template nucleic acids of an immortalization library. In various embodiments, the apparatus can be configured to perform nucleic acid sequencing (e.g., targeted gene sequencing, whole genome sequencing, or whole genome bisulfite sequencing).

The mixture of the reagents and template library may be presented to the apparatus through various conduits, examples of which include wells of a well plate (e.g., 96 well plate), a vial, a tube, and integrated fluidic circuits. As such, the apparatus may have an opening (e.g., a slot, a cavity, an opening, a sliding tray) that can receive the container including the reagent test sample mixture and perform a reading. Examples of an apparatus include one or more of a sequencer, an incubator, plate reader (e.g., a luminescent plate reader, absorbance plate reader, fluorescence plate reader), a spectrometer, or a spectrophotometer. Example sequencers include Illumina sequencers (e.g., Illumina MiSeq platform), Solexa platform, HeliScope from Helicos Biosciences, Roche sequencing system 454, or ion torrent sequencing technology. Example sequencers can perform sequencing of the nucleic acids using any of next generation sequencing, sequencing using SOLiD technology, pyrosequencing, Sanger sequencing, sequencing 454, and ion torrent sequencing. Sequencing can further involve aligning sequence reads to a reference genome to determine alignment position information. For example, sequence reads derived from DNA can be aligned to a range of positions of a reference genome. Further details for aligning sequence reads to reference sequences is described in U.S. application Ser. No. 16/279,315, which is hereby incorporated by reference in its entirety. In various embodiments, an output file having SAM (sequence alignment map) format or BAM (binary alignment map) format may be generated and output for subsequent analysis.

Systems disclosed herein may further include a computer system, such as example computer 700 shown in FIG. 7B. The computer 700 includes at least one processor 702 coupled to a chipset 704. The chipset 704 includes a memory controller hub 720 and an input/output (I/O) controller hub 722. A memory 706 and a graphics adapter 712 are coupled to the memory controller hub 720, and a display 718 is coupled to the graphics adapter 712. A storage device 708, an input device 714, and network adapter 716 are coupled to the I/O controller hub 722. Other embodiments of the computer 700 have different architectures.

The storage device 708 is a non-transitory computer-readable storage medium such as a hard drive, compact disk read-only memory (CD-ROM), DVD, or a solid-state memory device. The memory 706 holds instructions and data used by the processor 702. The input interface 714 is a touch-screen interface, a mouse, track ball, or other type of pointing device, a keyboard, or some combination thereof, and is used to input data into the computer 700. In some embodiments, the computer 700 may be configured to receive input (e.g., commands) from the input interface 714 via gestures from the user. The graphics adapter 712 displays images and other information on the display 718. The network adapter 716 couples the computer 700 to one or more computer networks.

The computer 700 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 708, loaded into the memory 706, and executed by the processor 702. A module can be implemented as computer program code processed by the processing system(s) of one or more computers. Computer program code includes computer-executable instructions and/or computer-interpreted instructions, such as program modules, which instructions are processed by a processing system of a computer. Generally, such instructions define routines, programs, objects, components, data structures, and so on, that, when processed by a processing system, instruct the processing system to perform operations on data or configure the processor or computer to implement various components or data structures in computer storage. A data structure is defined in a computer program and specifies how data is organized in computer storage, such as in a memory device or a storage device, so that the data can accessed, manipulated, and stored by a processing system of a computer.

The types of computers 700 can vary depending upon the embodiment and the processing power required by the entity. In various embodiments, operations can be run in a single computer 700 or across multiple computers 700 communicating with each other through a network such as in a server farm. In various embodiments, the computers 700 can lack some of the components described above, such as graphics adapters 712, and displays 718.

### EXAMPLES

The following Examples are merely illustrative and are not intended to limit the scope or content of the invention in any way.

### Example 1. Methods of Amplifying and Preserving Analyte Libraries: ARes V1 Validation

Patient-derived clinical samples are finite, constraining the number of potential diagnostic analyses. The AReS (Archived Reference Sample) process was developed to amplify the mass of index-derived DNA library to serve as a sample reservoir from which assay development and clinical studies can be performed. The AReS process can increase the mass available for downstream testing by ˜50× (see FIG. 2B).

This Example describes methods to produce Archived Reference Samples (AReS). The methods allow for amplification and preservation of analyte libraries to allow for a wider variety of testing than permissible solely from the original source. This Example also provides validation data from multiple substudies which together indicate that AReS is equivalent to a sequencing replicate of an original library.

TABLE 1 provides an overview of the testing performed in this Example.

TABLE 1
Study Overview

| | Variables | Number of AReS Samples |
|---|---|---|
| Initial Condition | 1, 5, 10 and 20 ng input, | 7 Samples per condition, |
| study | 5, 10 and 15 PCR cycles | 56 Samples |
| | (5 ng only) | |
| Initial | 3 replicates done with 5 | 16 samples, 8 Cancer, 8 |
| Reproducibility | ng input and 10 PCR | Non cancer, 3 replicates |
| Study | cycles | each, 48 samples total |
| Input Unique | 8 samples run across | 8 |
| Rate Study | different input and PCR | |
| | cycle numbers | |
| Secondary | Samples run in triplicate | 24 Cancer and 24 Non |
| Reproducibility | to determine if AReS | Cancer, 144 samples total |
| Study | reactions give consistent | |
| | metrics | |
| AReS CLIA LDT | Original library vs AReS | 197 |
| Study | concordance and 5 | |
| | samples run 3X for both | |
| | Inter and Intra AReS | |
| | replicates | |
| UMI Study | 8 Libraries, 1 cell line | 8 |
| | and 7 cfDNA, all | |
| | containing UMIs | |

### ARES V1 Protocol

AReS V1 starts with the generation of nucleic acid library. Each library fragment is flanked by primers and quantified for both mass and purity. Mass determined fractions of each library are input into PCR reactions utilizing the flanking primers and amplified to generate sufficient product without introducing bias. The amplified product is then cleaned and tested for mass and purity. Importantly, the uniform amplification of the library produces a proportional sampling of the original library, preserving the balance of the heterogenous nucleic acid population.

### Detailed Protocol

Whole genome bisulfite libraries are used for AReS V1 amplification. Each library contains flanking primers which are complementary to Illumina flowcell binding motifs. All libraries of interest are tested for quality by qubit for concentration and by TapeStation to define fragment size. At least 5 ng of each library is aliquoted and normalized to 1 ng/μl. Libraries are input into a PCR reaction with the following setup.

A Standard Setup is shown in TABLE 2.

TABLE 2

| | Standard Setup |
|---|---|
| | (μl) |
| Kapa Uracil 2X (KK2801) | 25 |
| Forward and Reverse | 5 |
| primers 10 μM Total, | |
| (5 μM Each target) | |
| Sample (1 ng/μl) | 5 |
| PCR Grade H₂0 | 15 |
| Total Volume | 50 |

Example Primers are shown in TABLE 3.

TABLE 3

| Primer | | SEQ ID |
|---|---|---|
| Name | Sequence | No: |
| Primer 1 | AATGATACGGCGACCACCGA | 1 |
| Primer 2 | CAAGCAGAAGACGGCATACGAGAT | 2 |

A PCR Reaction is run as shown in TABLE 4.

TABLE 4

| | Temp (Cel) | Duration (sec) | |
|---|---|---|---|
| Denaturation | 95° | 30 | |
| Denaturation | 98° | 10 | Repeat 5, 10, 15, or |
| Annealing | 60° | 30 | 20 cycles |
| Extension | 72° | 60 | |
| Extension | 72° | 300 | |
| | Hold° | Hold | |

The PCR reactions are then cleaned in a 0.9×SPRI Clean up protocol.

Briefly, 45 μl of SPRI beads are added to the PCR reaction, the sample is mixed by pipetting 10 times, and then incubated for 10 minutes. The samples are then incubated on a DynaMag-96 magnet until the SPRI beads are completely pelleted. The supernatant is then removed and the beads are washed two times with 200 μl of 80% ethanol. The beads are allowed to dry until any residual ethanol has evaporated and then resuspended in 50 μl of TE. After incubating for 5 minutes the supernatant is aspirated and moved to labeled tubes. The AReS libraries are once again tested for quality by qubit for concentration and by TapeStation to define fragment size.

### AReS Optimization:

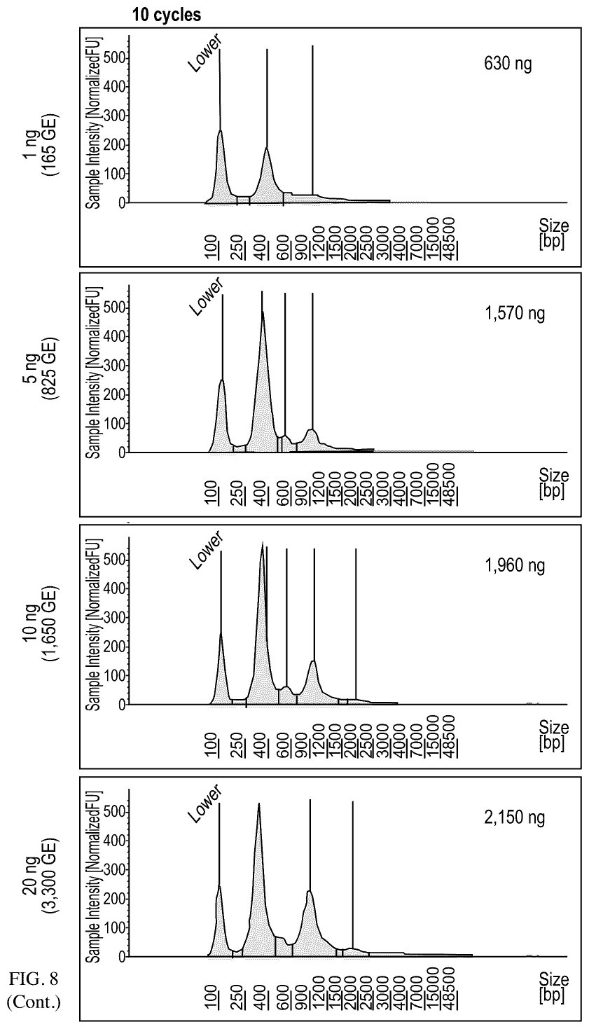

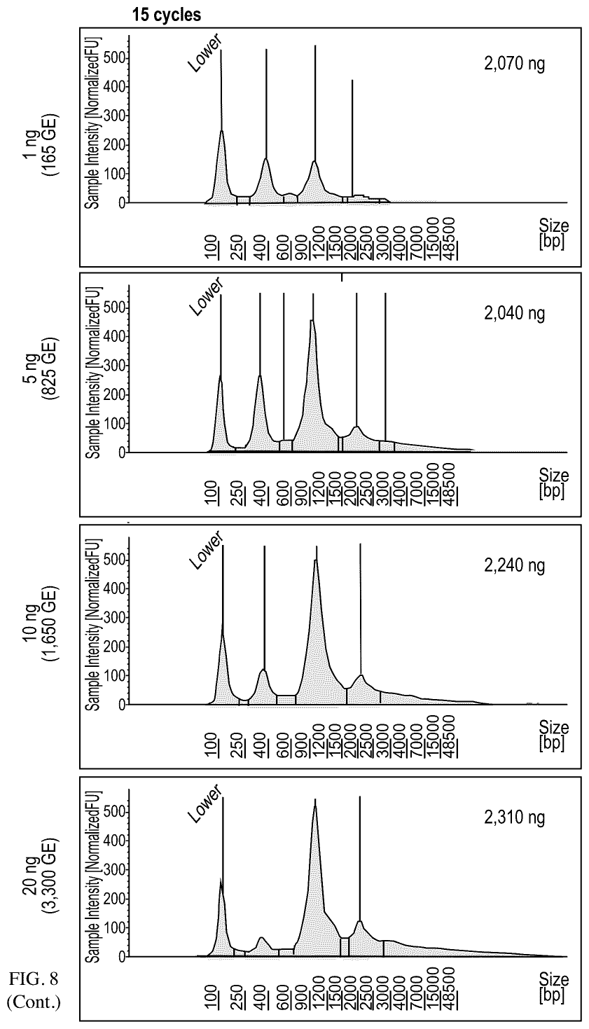

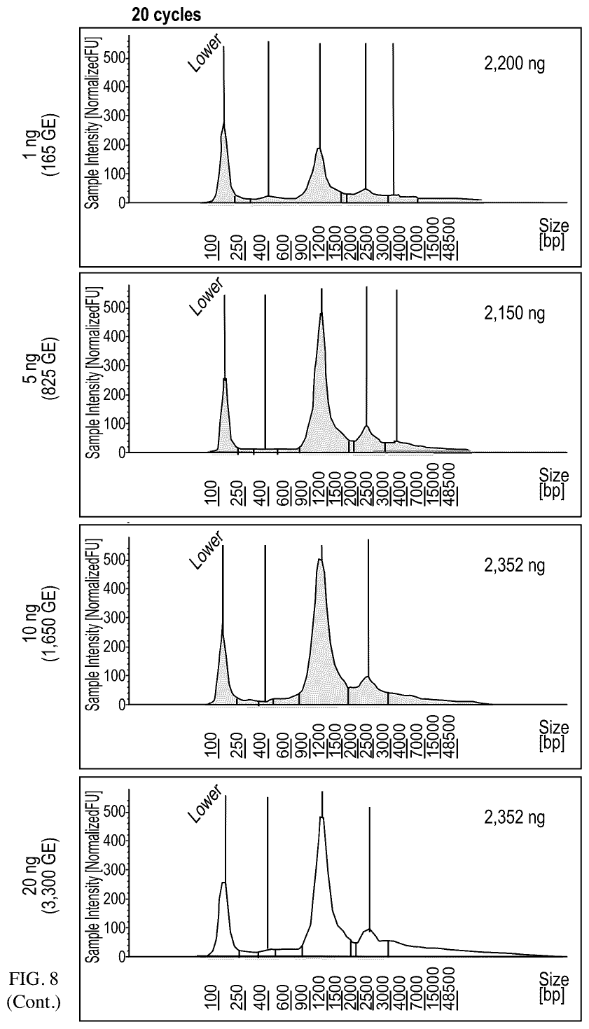

PCR Yield—The first AReS study was a titration of PCR amplification and DNA input conditions to determine whether additional amplifications were possible with starting libraries and the AReS Primers. All reactions successfully amplified, but size comparison via the TapeStation revealed that higher levels of PCR generated larger than expected product. As shown in FIG. 8, PCR products of different sizes were found depending upon the amount of input (1 ng, 5 ng, 10 ng, or 20 ng DNA) and the number of cycles (5, 10, 15, 20).

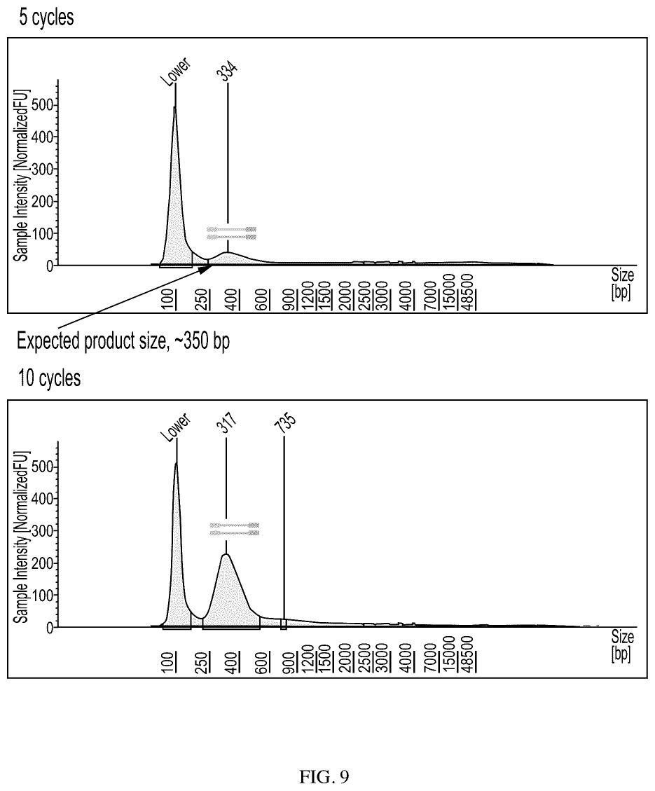

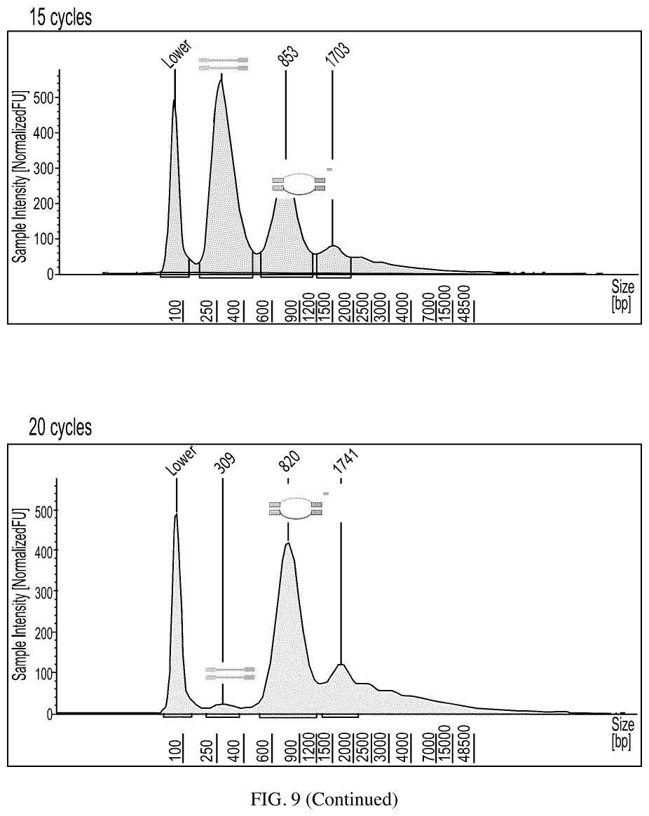

FIG. 9 provides annotated histograms for the 1 ng input trial, showing peaks of different-sized products. This larger than expected product was identified as “bubble product” which forms during PCR cycles when there is insufficient primer to initiate extension. As shown in FIG. 9, at cycles of 15 or more, “bubble product”, begins to form. Bubble product is depicted over the third peak as a spaced-apart DNA schematic. When DNA molecules go through denaturing and are unable to find the correct strand, non-complimentary DNA fragments which cannot hybridize become linked by their 5′ and 3′ adapters, producing a product with an inner bubble. Though this bubble product is not problematic in terms of sequencing data, it may be an indication of PCR bias. Lastly, this bubble product experiment identified the approximate maximum yield obtainable by the standard 0.1 μM primers in a 50 μl reaction as somewhere in the range of 2.5-3 μg.

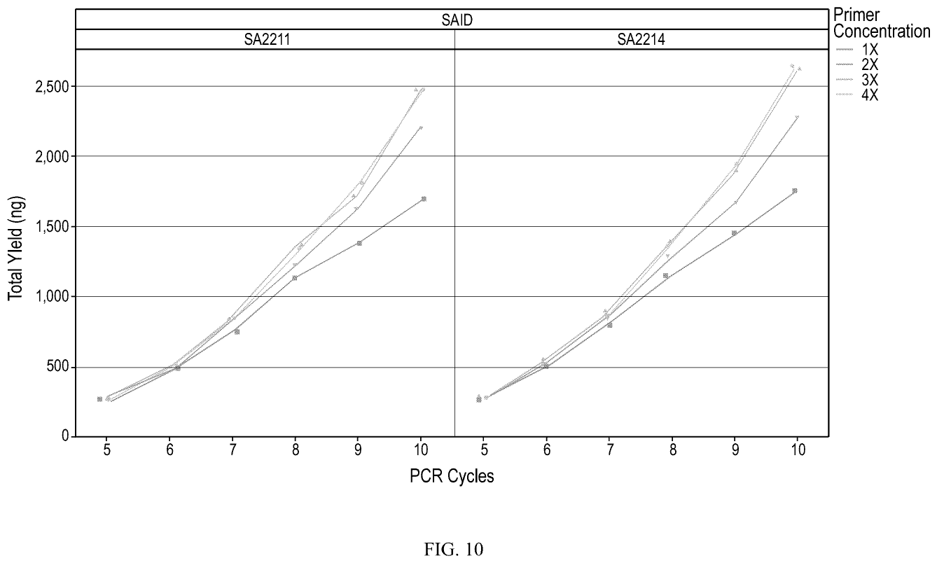

The effect of primer concentration on yield was also tested. Using 10 ng of input and 5-10 cycles PCR, primer concentration was varied. Primer concentrations of 1× (10 μM), 2× (20 μM), 3× (30 μM) or 4× (40 μM) were used. As shown in FIG. 10, maximum total yield was achieved using 3× (30 μM) primer concentration (yield was lowest with 1× primer concentration, increased with 2× primer concentration, and highest with 3× or 4× primer concentration).

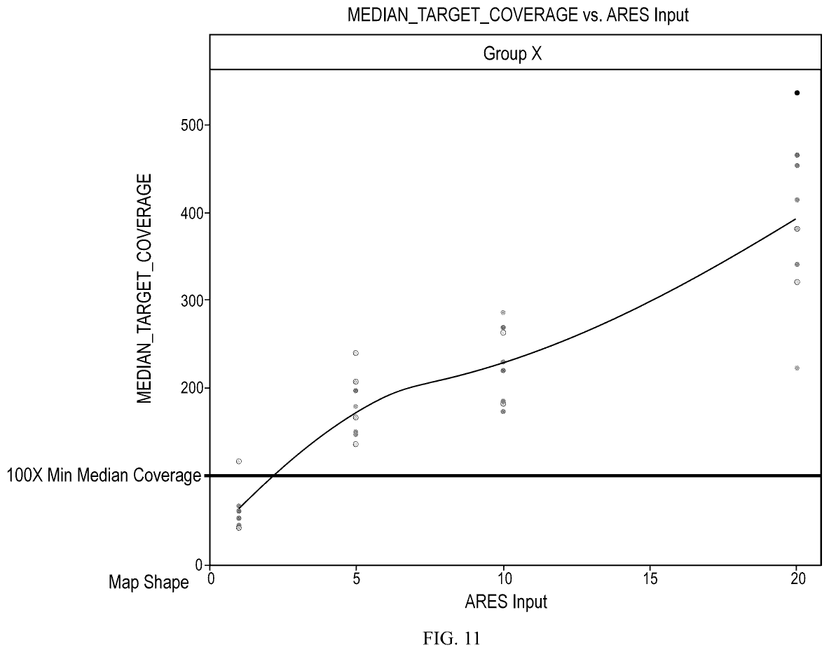

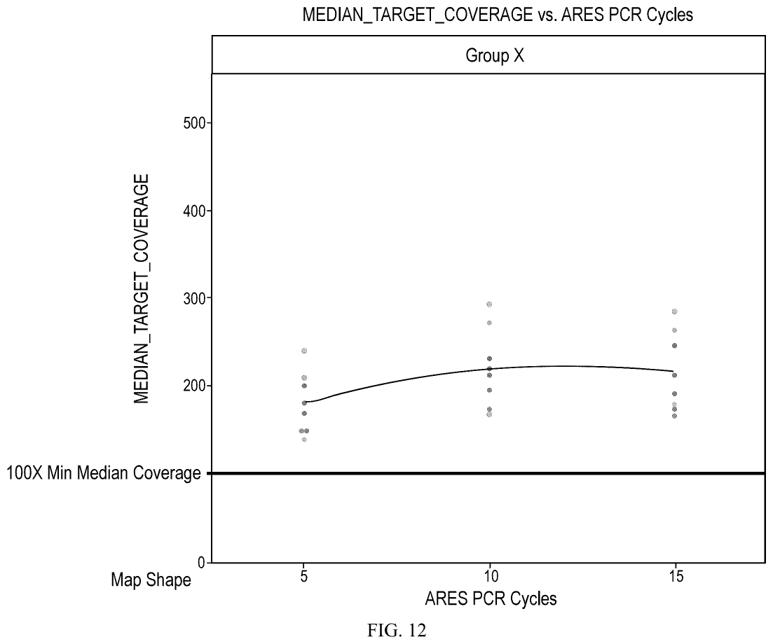

Amplified product was tested for target coverage. As shown in FIG. 11, inputs greater than 1 ng met median target coverage (100× minimum median coverage). As shown in FIG. 12, median target coverage was similar over PCR cycles, indicating that PCR cycle number has little impact on median target coverage.

### Unique Rate:

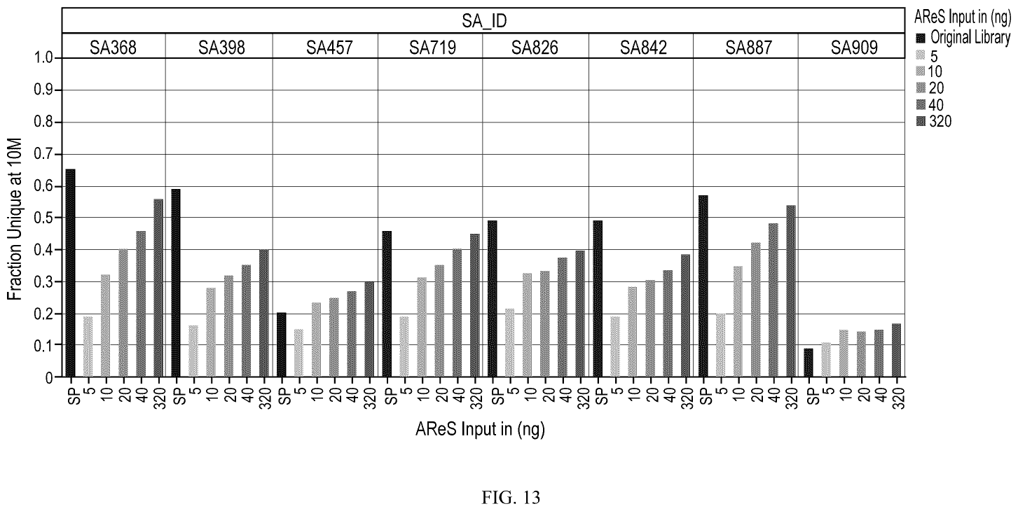

Initial sequencing data from AReS library had lower unique rates than original libraries. This may be due to a loss of molecules through both the initial AReS aliquot and the PCR amplification process. To maximize the unique rate, a titration of AReS inputs were sequenced and the unique rate was compared to the original library (see FIG. 13). Although this study was not coverage normalized, and a number of factors could have impacted the amount of total reads for the original library or the AReS library, there is a clear trend showing that increasing input increases the sample unique rate. However, the effect plateaus beginning at about 40 ng input. Increasing input from 40 to 320 ng AReS is an 8 fold increase in mass, but it only produces a 5-10% increase in unique rate (see FIG. 13). To strike a balance between initial input and maximizing unique rate, a 40 ng input with 8 PCR cycles was selected to create AReS libraries for further experiments. These conditions conserve the original library while maximizing the unique rate.

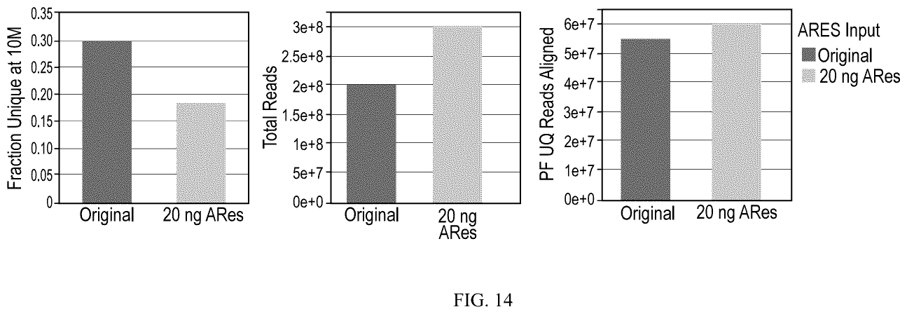

While AReS libraries have a lower unique rate, as shown in FIG. 13, historical data have shown that AReS samples having greater than 10% fraction unique at 10M usually pass median target coverage (MTC) quality control. Further, with additional sequencing AReS libraries can produce the same number of total unique reads as an original library. For example, as shown in FIG. 14, an AReS library created with 20 ng input showed a lower fraction unique at TOM than did the original library (left hand graph). However, when additional reads are produced through additional sequencing (middle graph), it can be seen in the right hand graph that the AReS library contained more unique reads than did the original library. This indicates that, while unique reads are masked by a higher duplicate rate, AReS libraries contain the same complexity that we observe in original libraries.

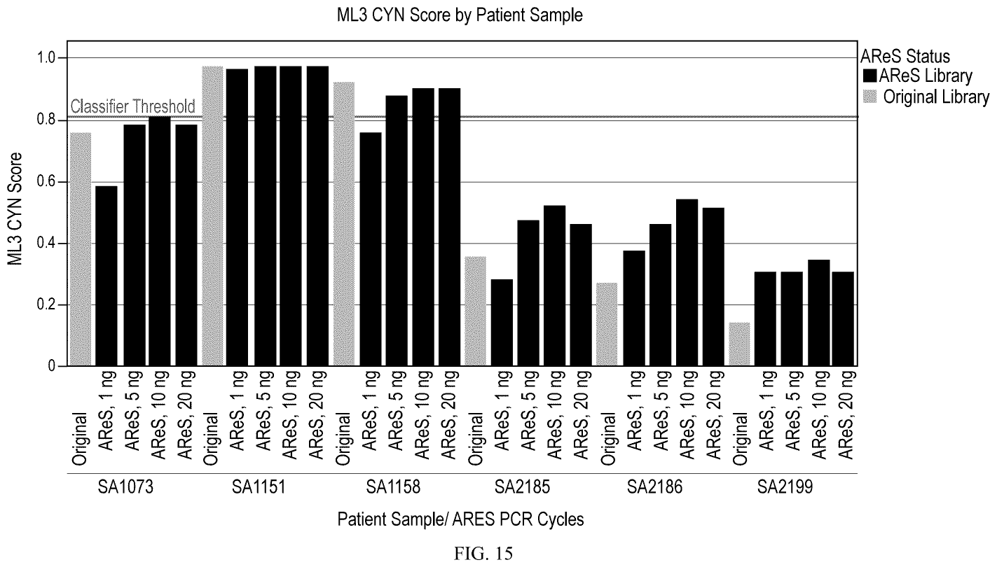

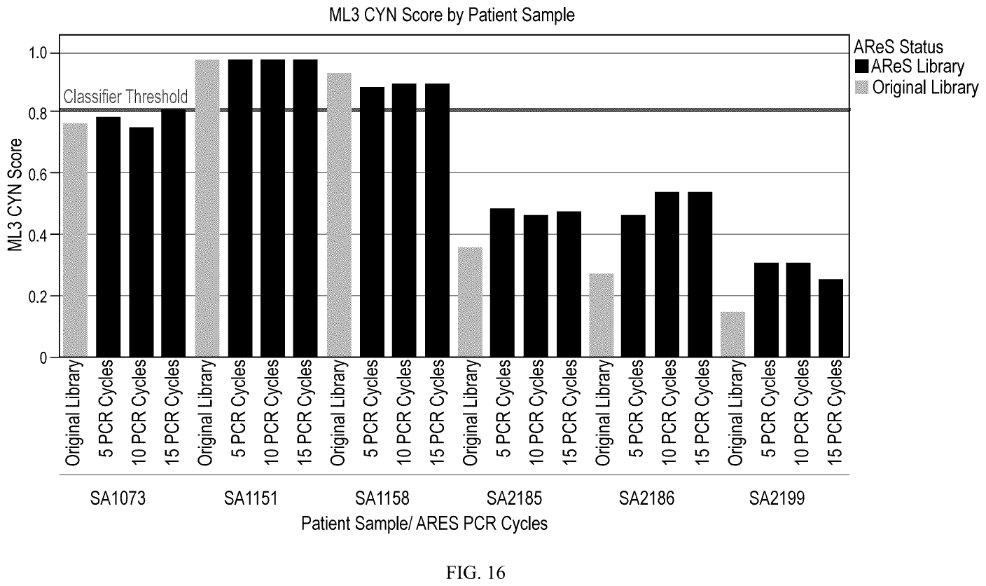

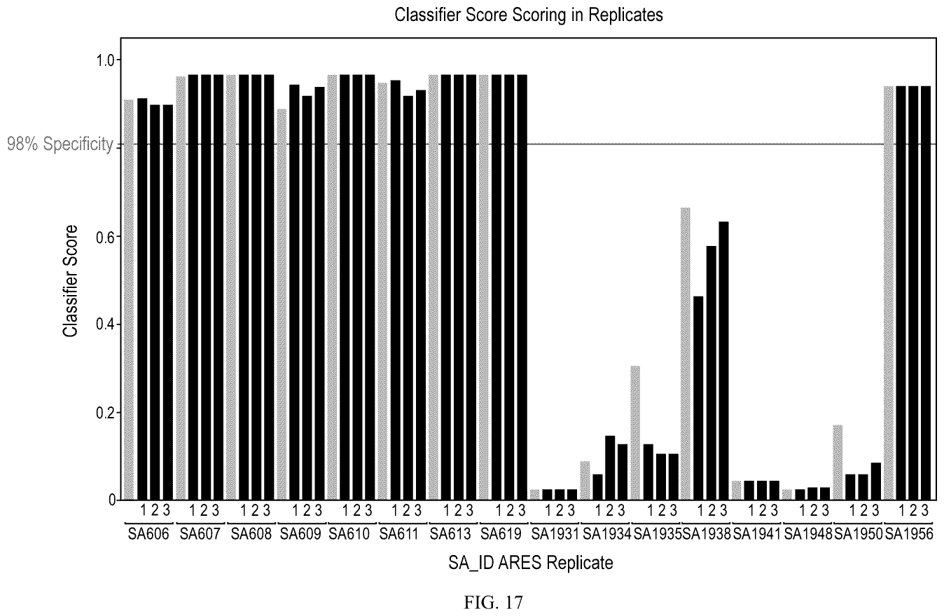

Next, classifier scores were measured and compared. As shown in FIG. 15, classifier scores are consistent across inputs (compare cancer and non-cancer for 5 ng, 10 ng, and 15 ng inputs) and across PCR cycles (FIG. 16) and inputs (FIG. 16). Further, as shown in FIG. 17, amplified samples demonstrated concordant classifier scores across replicates. For each patient sample (SA_ID), the control (standard process) is shown to the left (light colored bar) and the three (3) AReS replicate samples are shown to the right (dark bars 1, 2, and 3).

### AReS Meta Analysis:

Sequencing analysis of the numerous AReS studies was performed, including an initial condition study, initial input study, unique rate study, two reproducibility studies, and CLIA analytical validation study. In sum, this work contained 536 AReS libraries from 321 patient derived DNA samples.

### PyMHap Metrics

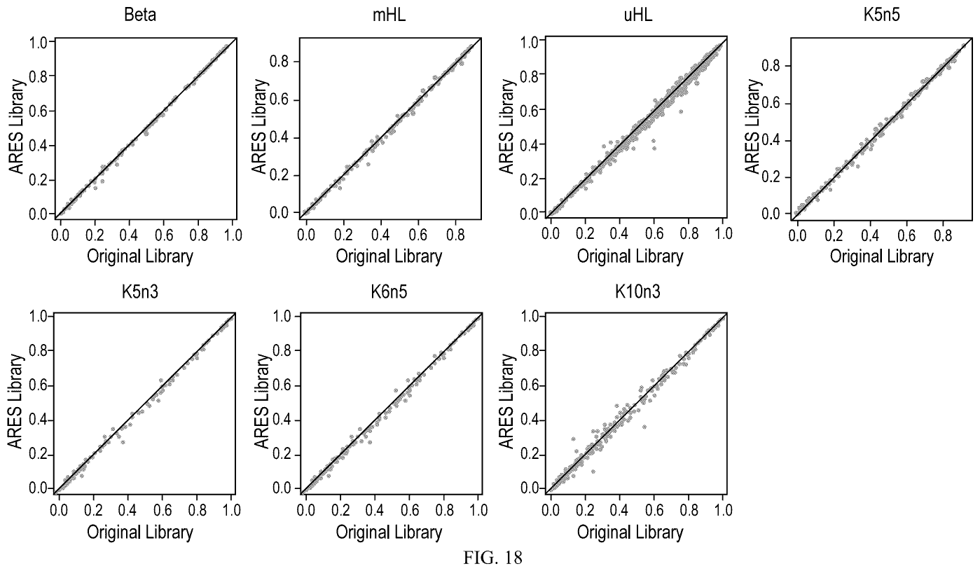

PyMHap metrics are methylation scoring calculations used to quantify the methylation states of individual sequencing reads. These methylation metrics are consolidated across reads to formulate the data necessary for classifier scoring. The PyMHap metrics of AReS libraries are highly concordant to those of original libraries across all of the CGIs of a 4,059 CGI panel (FIG. 18). This is further evidence that the lower unique rate seen in AReS libraries is reflective of a higher duplication rate but not any significant bias. When comparing the original libraries to AReS the PyMHap metrics across all samples the PyMHap metrics are over 98% concordant by Pearson's correlation to the original libraries across all reactions.

### Cancer Yes/No (CYN) Classifier Score:

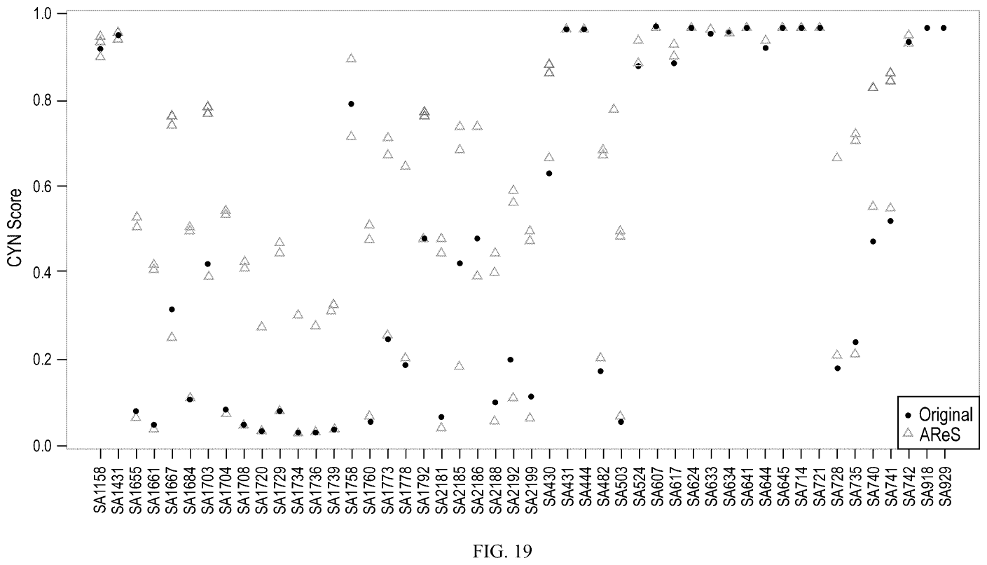

All of the available AReS libraries were compared to their original libraries using a cancer yes/no determining, fixed, multi-layered logistic regression-based machine learning derived algorithm. Cancer classification was concordant between original and AReS libraries for >97% of AReS samples (see FIG. 19).

### Ares UMI Study:

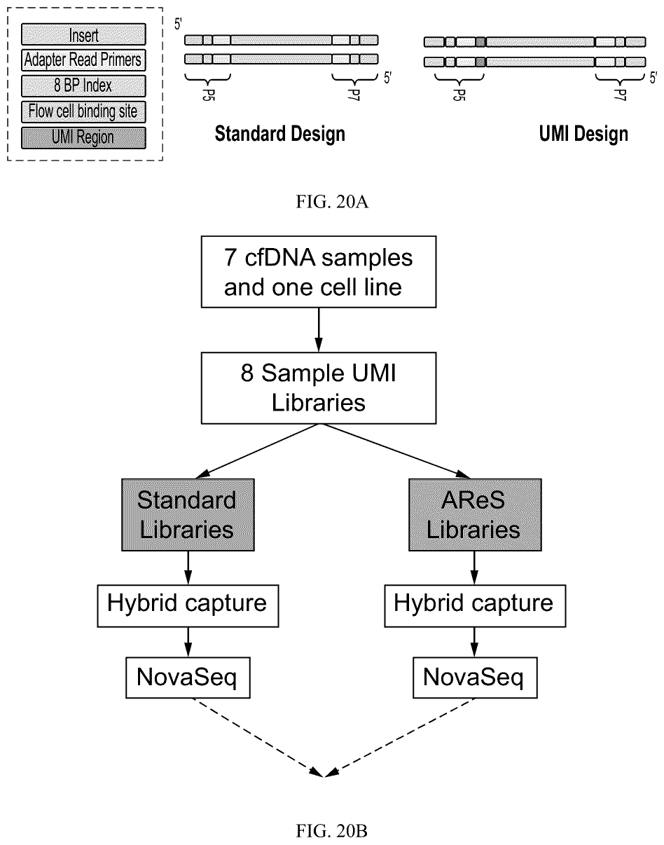

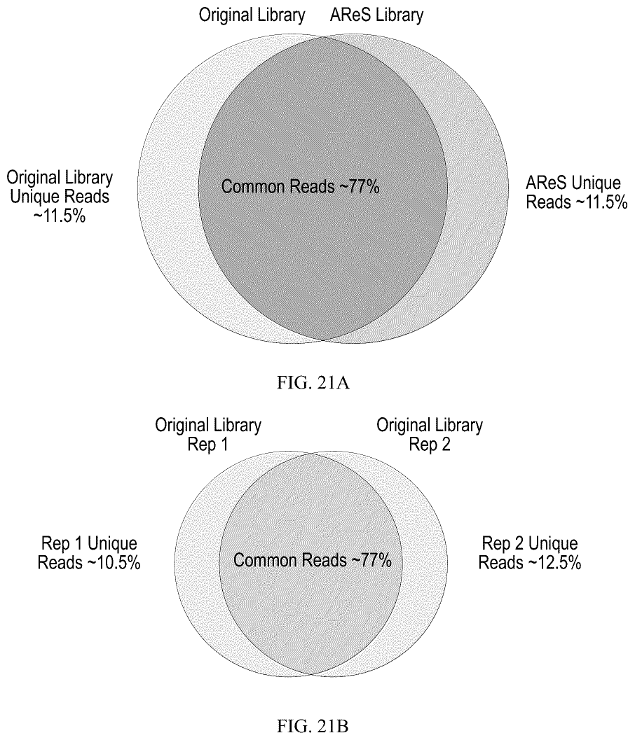

Unique molecular indices (UMIs) were incorporated into bisulfite treated cfDNA libraries. These UMIs contained random 9 bp sequences which barcoded individual strands of cfDNA. AReS libraries were generated from the original libraries and both sets were then enriched with the 4,059 hybrid capture probe set prior to sequencing (see FIGS. 20A and 20B). All libraries were downsampled to a uniform 50M on target reads and the UMI barcoded reads were compared between the 8 original libraries and their 8 AReS daughter libraries. Across the 8 samples ˜77% of reads were common between the original and AReS libraries (see FIG. 21A). To contextualize this relationship, the hybrid capture pools were sequenced a second time and the UMI barcoded reads were compared again between replicates. Importantly, resequencing a hybrid capture pool is the maximally identical data possible in the assay. When the original library replicates were compared the first and second replicate similarly shared ˜77% of reads (see FIG. 21B). Read frequency concordance of common reads between the original and AReS libraries was also within a few percent of read frequency concordance of common reads between original library replicates (see TABLE 5).

TABLE 5
Pearson correlation between original and AReS
libraries and Original library replicates

| | Original | Original |
|---|---|---|
| | Library vs | Library Rep 1 |
| | AReS | vs Rep 2 |
| | concordance | concordance |
| exDNA_848 | 75% | 77% |
| exDNA_196 | 63% | 67% |
| exDNA_400 | 76% | 79% |
| exDNA_224 | 82% | 83% |
| exDNA_040 | 82% | 83% |
| exDNA_209 | 84% | 85% |
| exDNA_349 | 88% | 89% |
| exDNA_649 | 95% | 96% |

In sum, these data indicate that AReS libraries are highly similar to original libraries, the equivalent of sequencing replicates.

In view of the foregoing, AReS has been found to be highly representative of the original library. As it is the equivalent of a technical replicate, it can be used for either technical or clinical development. While AReS libraries do have a lower unique rate, rarer reads are present at a lower frequency and can be identified through additional sequencing. There is no evidence of detectable amplification bias either through skews in methylation or ML metrics or in read composition. Incorporating UMIs into libraries prior to AReS enables nearly 100% deduplication which can eliminate duplicative effects produced by the AReS process.

Accordingly, this Example shows that the AReS V1 protocol can be used to produce an archived reference sample having consistent classifier scoring when compared to original (cfDNA) libraries, which can provide multiple nucleic acid library samples from the same source over time.

### Example 2. Methods of Amplifying and Preserving Analyte Libraries: AReS V2

### AReS V2 Protocol

In the AReS V2 method, depicted as Method 2 on FIG. 2, analyte libraries (e.g., DNA, RNA, or protein) are tagged with moieties amenable to covalent binding to a solid support (e.g., paramagnetic beads).

The immobilized libraries are preserved on the solid support as a permanent template. The immobilized library may be made available for bioassays either through using the bound library as a template to amplify a clone library in solution or by directly probing regions of interest on the library solid support complex. These methods allow for experimentation without consuming the bound library.

AReS V2 libraries are tagged with moieties such as phosphate or amino groups which can be used for covalent binding. Nucleic acid libraries are flanked by primers and quantified for both mass and purity. The tagged libraries are bound to a solid support through chemistries (e.g. tosylate coupling, carbodiimide crosslinking, Click Chemistry, etc.). Barcodes or standards of known quantity and composition may be introduced to the library either before or during the reaction to track the efficiency of covalent binding and serve as reference marker for future byproducts. Bound library solid support complexes are washed and preserved in a stable buffer (e.g. 0.1% sodium azide in TE) for long term storage. The bound library may be made available for analysis through processes such as using the flanking primers to amplify off a clone library into solution. The supernatant is removed and the original solid support complex is washed and returned to the buffer for storage.

### Detailed Protocol

Whole genome bisulfite libraries that have been amplified with flanking primers complimentary to Illumina flowcell binding motifs terminated with a 5′-phosphate group are used for AReS V2. These libraries are resuspended in d₂H₂O. A barcoded standard nucleic acid also terminated with a 5′-phosphate group is also utilized in the bead binding process. 200 μl of Carboxy functionalized paramagnetic beads are washed two times with Bioclone suspension buffer to remove any interfering compounds. The paramagnetic beads are resuspended in 200 μl coupling buffer and combined with the whole genome bisulfite library and 10 μM of standard, and the entire solution is mixed gently overnight at 50° C. in a shaker. Samples are then separated using the DynaMag-96 magnet until the beads are completely pelleted, the supernatant is removed and the beads are washed three times with 200 μl of washing buffer and then two times with d₂H₂O.

Alternatively, whole genome bisulfite libraries are amplified with flanking primers comprising amine tags are combined with EDC and carboxylic acid tagged magnetic beads to attach the magnetic beads to the primers by EDC chemistry. Alternatively, a BioClones BcMag kit can be used.

The beads are then resuspended in 20 μl TE buffer and run in a PCR reaction as follows:

A Standard Setup is shown in TABLE 6.

TABLE 6

| | Standard Setup |
|---|---|
| | (μl) |
| Kapa Uracil 2X (KK2801) | 25 |
| Forward and Reverse | 5 |
| primers 10 μM Total, | |
| (5 μM Each target) | |
| AReS Bead Mix | 20 |
| Total Volume | 50 |

Example Primers are shown in TABLE 7.

TABLE 7

| Primer | | SEQ ID |
|---|---|---|
| Name | Sequence | No: |
| Primer 1 | AATGATACGGCGACCACCGA | 1 |
| Primer 2 | CAAGCAGAAGACGGCATACGAGAT | 2 |

A PCR Reaction is run as shown in TABLE 8.

TABLE 8

| | Temp (Cel) | Duration (sec) | |
|---|---|---|---|
| Denaturation | 95° | 30 | |
| Denaturation | 98° | 10 | Repeat 2 cycles |
| Annealing | 60° | 30 | |
| Extension | 72° | 60 | |
| Extension | 72° | 300 | |
| | Hold° | Hold | |

The PCR reaction supernatants are then separated using the DynaMag-96 magnet until the beads are completely pelleted. While the supernatant is being cleaned by SPRI, the beads are washed with buffer. The PCR reactions are then cleaned in a 0.9×SPRI Clean up. 45 μl of SPRI beads are added to the PCR reaction supernatant, the sample is mixed by pipetting ten times and then incubated for 10 minutes. The samples are then incubated on a DynaMag-96 magnet until the SPRI beads have completely pelleted. The supernatant is then removed and the beads are washed two times with 200 μl of 80% ethanol. The beads are allowed to dry until any residual ethanol has evaporated and then resuspended in 50 μl of TE buffer. After incubating for 5 minutes the supernatant is removed into labeled tubes. The AReS libraries are once again tested for quality by qubit for concentration and by TapeStation to define fragment size. The AReS bound beads are then washed three times with 200 μl of washing buffer and then two times with d₂H₂O.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of emulating or conducting a human clinical study that uses a plurality of human subjects to evaluate a diagnostic parameter, wherein the diagnostic parameter comprises the methylation state of one or more nucleotides in the nucleic acid, the method comprising:
providing a portion of an archived reference sample (AReS) library of amplified nucleic acid representative of the cfDNA from the plurality of human subjects, wherein the library was prepared by a process comprising obtaining the cfDNA from the plurality of human subjects; subjecting the cfDNA to a cytosine deamination step, preparing an original library, and performing an amplification reaction to produce an excess of amplified library, thereby forming the AReS library;
assaying the portion of the AReS library with an assay for the diagnostic parameter to generate an output, wherein the output comprises sequencer data,
wherein the methylation state of the one or more nucleotides in the nucleic acid, as indicated by the sequencer data, correlates with presence or absence of a condition in each of the plurality of human subjects, wherein the condition is cancer; and comparing the sequencer data to a reference standard to determine sensitivity or specificity of the diagnostic parameter.

2. The method of claim 1, wherein the nucleic acid of the AReS library and/or the original library of each of the plurality of human subjects comprises a unique identifier.

3. The method of claim 2, wherein the unique identifier allows for the identification of the source of the sample, such that the output can be correlated with whether or not the human subject has cancer, thereby to correlate the diagnostic parameter with the presence or absence of cancer in the subject.

4. The method of claim 1, the method further comprises assaying a second diagnostic parameter, comprising the steps of (1) assaying a second portion of an AReS library with an assay for the second diagnostic parameter to generate a second output, wherein the second output correlates with presence or absence of cancer or a second condition in each of the plurality of human subjects; and (2) comparing the second output to a second reference standard to determine sensitivity or specificity of the second diagnostic parameter.

5. The method of claim 4, further comprising repeating steps (1) and (2) with at least one additional portion of the AReS library to determine sensitivity or specificity for at least one additional diagnostic parameter.

6. The method of claim 4, wherein the diagnostic parameter and second diagnostic parameter are the same.

7. The method of claim 5, wherein two or more of the diagnostic parameter, second diagnostic parameter, and at least one additional diagnostic parameter are the same.

8. The method of claim 4, wherein the second diagnostic parameter is different from the diagnostic parameter.

9. The method of claim 4, wherein the second output is the same as the output.

10. The method of claim 4, wherein the second output is different from the output.

11. The method of claim 4, wherein the second output comprises a fluorescent signal.

12. The method of claim 4, wherein the output and/or the second output is associated with the presence of the condition.

13. The method of claim 4, wherein the output and/or the second output is associated with the absence of the condition.

14. The method of claim 5, wherein the second diagnostic parameter or the at least one additional diagnostic parameter comprises a methylation state of one or more nucleotides in the cfDNA or the presence of a mutation in the cfDNA.

15. A method of emulating or conducting a human clinical study that uses a plurality of human subjects to evaluate a diagnostic parameter, wherein the diagnostic parameter comprises the methylation state of one or more nucleotides in the nucleic acid, the method comprising:
providing a clone library of amplified nucleic acid from each of the plurality of human subjects, wherein at least one clone library was prepared by a process comprising:
obtaining cell free DNA (cfDNA) from a human subject;
forming a immortalized library of template nucleic acid, wherein the immortalized library comprises a component or means for recovering the template nucleic acid; and
amplifying the immortalized library of template nucleic acid to form the at least one clone library of amplified nucleic acid;
assaying the clone libraries with an assay for the diagnostic parameter to generate an output, wherein the output comprises sequencer data, and wherein the methylation state of the one or more nucleotides in the nucleic acid, as indicated by the sequencer data, correlates with presence or absence of a condition in each of the plurality of human subjects, wherein the condition is cancer; and
comparing the sequencer data to a reference standard to determine sensitivity or specificity of the diagnostic parameter.

16. The method of claim 15, wherein the nucleic acid of the immortalized library and/or the clone library of each of the plurality of human subjects comprises a unique identifier.

17. The method of claim 16, wherein the unique identifier allows for the identification of the source of the sample, such that the output can be correlated with whether or not the human subject has the condition, thereby to correlate the diagnostic parameter with the condition or lack of condition in the subject.

18. The method of claim 15, further comprising assaying a second diagnostic parameter, comprising the steps of:
(1) assaying a second clone library with an assay for the second diagnostic parameter to generate a second output, wherein the second clone library was generated by amplifying the immortalized library, and
(2) determining whether the output correlates with presence or absence of the condition or a second condition in each of the plurality of human subjects.

19. The method of claim 18, further comprising repeating steps (1) and (2) with at least one additional clone library to determine whether the output correlates with presence or absence of the condition, the second condition, or at least one addition condition for at least one additional diagnostic parameter.

20. The method of claim 18 wherein the second diagnostic parameter is the same as the diagnostic parameter.

21. The method of claim 18, wherein the second diagnostic parameter is different from the diagnostic parameter.

22. The method of claim 19, wherein the at least one additional diagnostic parameter is the same as the diagnostic parameter and the second diagnostic parameter.

23. The method of claim 18, wherein the second output is the same as the output.

24. The method of claim 18, wherein the second output is different from the output.

25. The method of claim 18, wherein the second output comprises a fluorescent signal.

26. The method of claim 18, wherein the output and/or the second output is associated with the presence of the condition.

27. The method of claim 18, wherein the output and/or the second output is associated with the absence of the condition.

28. The method of claim 19, wherein the second diagnostic parameter or the at least one additional diagnostic parameter comprises a methylation state of one or more nucleotides in the cfDNA or the presence of a mutation in the cfDNA.

29. The method of claim 15, wherein the component for the recovery of the template nucleic acid comprises a solid support or a moiety that binds to a solid support.

30. The method of claim 15, wherein the component for the recovery of the template nucleic acid comprises a detectable moiety.


SEQUENCE LISTING (2 sequences)

SEQ ID NO: 1 - DNA, 20 bp, synthetic construct
  source 1..20: mol_type = other DNA
aatgatacggcgaccaccga

SEQ ID NO: 2 - DNA, 24 bp, synthetic construct
  source 1..24: mol_type = other DNA
caagcagaagacggcatacgagat